SEQUENCE LISTING (128 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2-15 - skipped (14 entries)

SEQ ID NO: 16 - protein, 7 aa, synthetic construct
LVSKLDS

SEQ ID NO: 17-19 - skipped (3 entries)

SEQ ID NO: 20 - protein, 111 aa, synthetic construct
EVQLQQSGPELVKPGASVKISCKTSGFTFTEYSMHWVKQSHGKSLEWIGGINPNNGGTSYNQKFKGKATLTVDKSSSTAYMELRSLTSEDSAVYYCARESWGQGTTLTVSS

SEQ ID NO: 21 - protein, 5 aa, synthetic construct
EYSMH

SEQ ID NO: 22 - protein, 17 aa, synthetic construct
GINPNNGGTSYNQKFKG

SEQ ID NO: 23 - skipped

SEQ ID NO: 24 - protein, 112 aa, synthetic construct
DVVMTQTPLTLSVTIGQPASISCKSSQSLLHSDGKTYLNWLLQRPGQSPKRLIYLVSKLDSRIPDRFTGSGSGTDFTLKISRVEAEDLGVYYCWQGTHFPHTFGAGTKLELK

SEQ ID NO: 25 - protein, 16 aa, synthetic construct
KSSQSLLHSDGKTYLN

SEQ ID NO: 26 - skipped

SEQ ID NO: 27 - protein, 9 aa, synthetic construct
WQGTHFPHT

SEQ ID NO: 28 - DNA, 333 bp, synthetic construct
  source 1..333: mol_type = other DNA
gaggtgcagctgcagcagagcggccccgaactggtgaagcccggcgcctccgtgaagatcagctgcaagaccagcggcttcaccttcaccgagtacagcatgcactgggtgaagcagtcccacggcaagtctctggagtggatcggcggcatcaaccccaacaacggcggcaccagctacaaccagaagttcaagggcaaggccacactgacagtggacaagagcagcagcaccgcctacatggagctgagatctctgaccagcgaggactccgccgtgtactactgcgccagagagagctggggccaaggcaccacactgaccgtgagcagc

SEQ ID NO: 29 - DNA, 336 bp, synthetic construct
  source 1..336: mol_type = other DNA
gacgtggtgatgacccagacccctctgacactgagcgtgaccatcggacagcccgcctccatcagctgcaaaagctcccagtctctgctgcacagcgacggcaagacctatctgaactggctgctgcagagacccggccaaagccccaagaggctgatctatctggtgagcaagctggactctagaatccccgacagatttaccggcagcggcagcggcacagactttacactgaagatctctagagtggaggccgaagatctgggcgtgtactactgctggcaaggcacccacttccctcacaccttcggcgccggcaccaagctggagctgaag

SEQ ID NO: 30 - protein, 111 aa, synthetic construct
EVQLVQSGAEVKKPGATVKISCKTSGFTFTEYSMHWVQQAPGKGLEWIGGINPNNGGTSYNQKFKGRVTITVDKSTDTAYMELSSLRSEDTAVYYCARESWGQGTTVTVSS

SEQ ID NO: 31-33 - skipped (3 entries)

SEQ ID NO: 34 - protein, 112 aa, synthetic construct
DVVMTQSPLSLPVTPGEPASISCKSSQSLLHSDGKTYLNWLLQKPGQSPKRLIYLVSKLDSRVPDRFSGSGSGTDFTLKISRVEAEDVGVYYCWQGTHFPHTFGQGTKLEIK

SEQ ID NO: 35-37 - skipped (3 entries)

SEQ ID NO: 38 - DNA, 333 bp, synthetic construct
  source 1..333: mol_type = other DNA
gaagtgcagctggtgcagagcggcgcggaagtgaaaaaaccgggcgcgaccgtgaaaattagctgcaaaaccagcggctttacctttaccgaatatagcatgcattgggtgcagcaggcgccgggcaaaggcctggaatggattggcggcattaacccgaacaacggcggcaccagctataaccagaaatttaaaggccgtgtgaccattaccgtggataaaagcaccgataccgcgtatatggaactgagcagcctgcgtagcgaagataccgcggtgtattattgcgcgcgtgaaagctggggccagggcaccaccgtgaccgtgagcagc

SEQ ID NO: 39 - DNA, 336 bp, synthetic construct
  source 1..336: mol_type = other DNA
gatgtggtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgaaacgtctgatttatctggtgagcaaactggatagccgtgtgccggatcgttttagcggcagcggcagcggcaccgattttaccctgaaaattagccgtgtggaagcggaagatgtgggcgtgtattattgctggcagggcacccattttccgcatacctttggccagggcaccaaactggaaattaaa

SEQ ID NO: 40 - protein, 111 aa, synthetic construct
EVQLVQSGAEVKKPGATVKISCKTSGFTFTEYSMHWVQQAPGKGLEWIGGINPNNGGTSYNQKFKGRVTITVDTSTDTAYMELSSLRSEDTAVYYCARESWGQGTTVTVSS

SEQ ID NO: 41-43 - skipped (3 entries)

SEQ ID NO: 44 - protein, 112 aa, synthetic construct
DIVMTQSPLSLPVTPGEPASISCKSSQSLLHSDGKTYLNWLLQKPGQSPKRLIYLVSKLDSRVPDRFSGSGSGTDFTLKISRVEAEDVGVYYCWQGTHFPHTFGQGTKLEIK

SEQ ID NO: 45-47 - skipped (3 entries)

SEQ ID NO: 48 - DNA, 333 bp, synthetic construct
  source 1..333: mol_type = other DNA
gaagtgcagctggtgcagagcggcgcggaagtgaaaaaaccgggcgcgaccgtgaaaattagctgcaaaaccagcggctttacctttaccgaatatagcatgcattgggtgcagcaggcgccgggcaaaggcctggaatggattggcggcattaacccgaacaacggcggcaccagctataaccagaaatttaaaggccgtgtgaccattaccgtggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 49 - DNA, 336 bp, synthetic construct
  source 1..336: mol_type = other DNA
gatattgtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgaaacgtctgatttatctggtgagcaaactggatagccgtgtgccggatcgttttagcggcagcggcagcggcaccgattttaccctgaaaattagccgtgtggaagcggaagatgtgggcgtgtattattgctggcagggcacccattttccgcatacctttggccagggcaccaaactggaaattaaa

SEQ ID NO: 50 - protein, 111 aa, synthetic construct
EVQLVQSGAEVKKPGATVKISCKTSGFTFTEYSMHWVQQAPGKGLEWMGGINPNNGGTSYNQKFKGRVTITVDTSTDTAYMELSSLRSEDTAVYYCARESWGQGTTVTVSS

SEQ ID NO: 51-53 - skipped (3 entries)

SEQ ID NO: 54 - protein, 112 aa, synthetic construct
DVVMTQSPLSLPVTPGEPASISCKSSQSLLHSDGKTYLNWLLQKPGQSPQRLIYLVSKLDSRVPDRFSGSGSGTDFTLKISRVEAEDVGVYYCWQGTHFPHTFGQGTKLEIK

SEQ ID NO: 55-57 - skipped (3 entries)

SEQ ID NO: 58 - DNA, 333 bp, synthetic construct
  source 1..333: mol_type = other DNA
gaagtgcagctggtgcagagcggcgcggaagtgaaaaaaccgggcgcgaccgtgaaaattagctgcaaaaccagcggctttacctttaccgaatatagcatgcattgggtgcagcaggcgccgggcaaaggcctggaatggatgggcggcattaacccgaacaacggcggcaccagctataaccagaaatttaaaggccgtgtgaccattaccgtggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 59 - DNA, 336 bp, synthetic construct
  source 1..336: mol_type = other DNA
gatgtggtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgcagcgtctgatttatctggtgagcaaactggatagccgtgtgccggatcgttttagcggcagcggcagcggcaccgattttaccctgaaaattagccgtgtggaagcggaagatgtgggcgtgtattattgctggcagggcacccattttccgcatacctttggccagggcaccaaactggaaattaaa

SEQ ID NO: 60 - protein, 111 aa, synthetic construct
EVQLVQSGAEVKKPGATVKISCKVSGFTFTEYSMHWVQQAPGKGLEWMGGINPNNGGTSYNQKFKGRVTITVDTSTDTAYMELSSLRSEDTAVYYCARESWGQGTTVTVSS

SEQ ID NO: 61-63 - skipped (3 entries)

SEQ ID NO: 64 - protein, 112 aa, synthetic construct
DIVMTQSPLSLPVTPGEPASISCKSSQSLLHSDGKTYLNWLLQKPGQSPQRLIYLVSKLDSRVPDRFSGSGSGTDFTLKISRVEAEDVGVYYCWQGTHFPHTFGQGTKLEIK

SEQ ID NO: 65-67 - skipped (3 entries)

SEQ ID NO: 68 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgaacaacggcggcaccagctataaccagaaatttaaaggccgtgtgaccattaccgtggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 69 - DNA, 336 bp, synthetic construct
  source 1..336: mol_type = other DNA
gatattgtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgcagcgtctgatttatctggtgagcaaactggatagccgtgtgccggatcgttttagcggcagcggcagcggcaccgattttaccctgaaaattagccgtgtggaagcggaagatgtgggcgtgtattattgctggcagggcacccattttccgcatacctttggccagggcaccaaactggaaattaaa

SEQ ID NO: 70 - protein, 111 aa, synthetic construct
EVQLVQSGAEVKKPGATVKISCKVSGFTFTEYSMHWVQQAPGKGLEWMGGINPNNGGTSYNQKFKGRVTITADTSTDTAYMELSSLRSEDTAVYYCARESWGQGTTVTVSS

SEQ ID NO: 71-73 - skipped (3 entries)

SEQ ID NO: 74 - protein, 112 aa, synthetic construct
DIVMTQSPLSLPVTPGEPASISCKSSQSLLHSDGKTYLNWLLQKPGQSPQLLIYLVSKLDSRVPDRFSGSGSGTDFTLKISRVEAEDVGVYYCWQGTHFPHTFGQGTKLEIK

SEQ ID NO: 75-77 - skipped (3 entries)

SEQ ID NO: 78 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgaacaacggcggcaccagctataaccagaaatttaaaggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 79 - DNA, 336 bp, synthetic construct
  source 1..336: mol_type = other DNA
gatattgtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgcagctgctgatttatctggtgagcaaactggatagccgtgtgccggatcgttttagcggcagcggcagcggcaccgattttaccctgaaaattagccgtgtggaagcggaagatgtgggcgtgtattattgctggcagggcacccattttccgcatacctttggccagggcaccaaactggaaattaaa

SEQ ID NO: 80 - protein, 111 aa, synthetic construct
EVQLVQSGAEVKKPGATVKISCKVSGFTFTEYSMHWVQQAPGKGLEWMGGINPNQGGTSYNQKFKGRVTITADTSTDTAYMELSSLRSEDTAVYYCARESWGQGTTVTVSS

SEQ ID NO: 81 - skipped

SEQ ID NO: 82 - protein, 17 aa, synthetic construct
GINPNQGGTSYNQKFKG

SEQ ID NO: 83 - skipped

SEQ ID NO: 84 - protein, 112 aa, synthetic construct
DIVMTQSPLSLPVTPGEPASISCKSSQSLLHSDAKTYLNWLLQKPGQSPQRLIYLVSKLDSRVPDRFSGSGSGTDFTLKISRVEAEDVGVYYCFQGTHFPHTFGQGTKLEIK

SEQ ID NO: 85 - protein, 16 aa, synthetic construct
KSSQSLLHSDAKTYLN

SEQ ID NO: 86 - skipped

SEQ ID NO: 87 - protein, 9 aa, synthetic construct
FQGTHFPHT

SEQ ID NO: 88 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgaaccagggcggcaccagctataaccagaaatttaaaggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 89 - DNA, 336 bp, synthetic construct
  source 1..336: mol_type = other DNA
gatattgtgatgacccagagcccgctgagcctgccggtgaccccgggcgaaccggcgagcattagctgcaaaagcagccagagcctgctgcatagcgatgcgaaaacctatctgaactggctgctgcagaaaccgggccagagcccgcagcgtctgatttatctggtgagcaaactggatagccgtgtgccggatcgttttagcggcagcggcagcggcaccgattttaccctgaaaattagccgtgtggaagcggaagatgtgggcgtgtattattgctttcagggcacccattttccgcatacctttggccagggcaccaaactggaaattaaa

SEQ ID NO: 90 - protein, 111 aa, synthetic construct
EVQLVQSGAEVKKPGATVKISCKVSGFTFTEYSMHWVQQAPGKGLEWMGGINPQNAGTSYNQKFKGRVTITADTSTDTAYMELSSLRSEDTAVYYCARESWGQGTTVTVSS

SEQ ID NO: 91 - skipped

SEQ ID NO: 92 - protein, 17 aa, synthetic construct
GINPQNAGTSYNQKFKG

SEQ ID NO: 93 - skipped

SEQ ID NO: 94 - protein, 112 aa, synthetic construct
DIVMTQSPLSLPVTPGEPASISCKSSQSLLHSDGKTYLNWLLQKPGQSPQRLIYLVSKLDSRVPDRFSGSGSGTDFTLKISRVEAEDVGVYYCFQGTHFPHTFGQGTKLEIK

SEQ ID NO: 95-97 - skipped (3 entries)

SEQ ID NO: 98 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgcagaacgcgggcaccagctataaccagaaatttaaaggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 99 - DNA, 336 bp, synthetic construct
  source 1..336: mol_type = other DNA
gatattgtgatgacccagagcccgctgagcctgccggtgaccccgggcgaaccggcgagcattagctgcaaaagcagccagagcctgctgcatagcgatggcaaaacctatctgaactggctgctgcagaaaccgggccagagcccgcagcgtctgatttatctggtgagcaaactggatagccgtgtgccggatcgttttagcggcagcggcagcggcaccgattttaccctgaaaattagccgtgtggaagcggaagatgtgggcgtgtattattgctttcagggcacccattttccgcatacctttggccagggcaccaaactggaaattaaa

SEQ ID NO: 100 - protein, 111 aa, synthetic construct
EVQLVQSGAEVKKPGATVKISCKVSGFTFTEYSMHWVQQAPGKGLEWMGGINPNQGGTSYNQKFQGRVTITADTSTDTAYMELSSLRSEDTAVYYCARESWGQGTTVTVSS

SEQ ID NO: 101 - skipped

SEQ ID NO: 102 - protein, 17 aa, synthetic construct
GINPNQGGTSYNQKFQG

SEQ ID NO: 103-107 - skipped (5 entries)

SEQ ID NO: 108 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgaaccagggcggcaccagctataaccagaaatttcagggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 109 - skipped

SEQ ID NO: 110 - protein, 111 aa, synthetic construct
EVQLVQSGAEVKKPGATVKISCKVSGFTFTEYSMHWVQQAPGKGLEWMGGINPQNAGTSYNQKFQGRVTITADTSTDTAYMELSSLRSEDTAVYYCARESWGQGTTVTVSS

SEQ ID NO: 111 - skipped

SEQ ID NO: 112 - protein, 17 aa, synthetic construct
GINPQNAGTSYNQKFQG

SEQ ID NO: 113-117 - skipped (5 entries)

SEQ ID NO: 118 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgcagaacgcgggcaccagctataaccagaaatttcagggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

SEQ ID NO: 119 - skipped

SEQ ID NO: 120 - protein, 111 aa, synthetic construct
EVQLVQSGAEVKKPGATVKISCKVSGFTFTEYSMHWVQQAPGKGLEWMGGINPNNGGTSYNQKFQGRVTITADTSTDTAYMELSSLRSEDTAVYYCARESWGQGTTVTVSS

SEQ ID NO: 121 - skipped

SEQ ID NO: 122 - protein, 17 aa, synthetic construct
GINPNNGGTSYNQKFQG

SEQ ID NO: 123-127 - skipped (5 entries)

SEQ ID NO: 128 - DNA, 333 bp, synthetic construct
  source 1..333: mol_type = other DNA
gaagtgcagctggtgcagagcggcgcggaagtgaaaaaaccgggcgcgaccgtgaaaattagctgcaaagtgagcggctttacctttaccgaatatagcatgcattgggtgcagcaggcgccgggcaaaggcctggaatggatgggcggcattaacccgaacaacggcggcaccagctataaccagaaatttcagggccgtgtgaccattaccgcggataccagcaccgataccgcgtatatggaactgagcagcctgcgtagcgaagataccgcggtgtattattgcgcgcgtgaaagctggggccagggcaccaccgtgaccgtgagcagc

(19) United States
(12) Patent Application Publication
(10) Pub. No.: US 2025/0145695 A1
(43) Pub. Date: May 8, 2025

(54) Humanized Anti-TDP-43 Binding Molecules and Uses Thereof

(71) Applicant: AC Immune SA, Lausanne (CH)

(72) Inventors: Tariq Afroz, Lausanne (CH); Romain Christian Ollier, Lausanne (CH); Mickaël Marc Pascal Audrain, Lausanne (CH)

(21) Appl. No.: 18/837,534

(22) PCT Filed: Feb. 16, 2023
(86) PCT No.: PCT/EP2023/053940

§ 371 Date: Aug. 9, 2024

(30) Foreign Application Priority Data
Feb. 16, 2022 (EP) 22157150.8
Jun. 6, 2022 (EP) 22177361.7
Jan. 23, 2023 (EP) 23152958.7
Jan. 24, 2023 (EP) 23153165.8

(51) Int. Cl. C07K 16/18; A61K 39/00

(52) U.S. Cl. CPC C07K 16/18; A61K 2039/505; C07K 2317/24; C07K 2317/76; C07K 2317/92; C07K 2317/94

(57) ABSTRACT

The present invention is in the field of transactive response DNA binding protein with a molecular weight of 43 kDa (TARDB or also TDP-43). The invention relates to humanized TDP-43 specific binding molecules, in particular to humanized anti-TDP-43 antibodies or antigen-binding fragment or a derivative thereof and uses thereof. The present invention provides means and methods to diagnose, prevent, alleviate and/or treat a disease, disorder and/or abnormality associated with TDP-43 aggregates including but not limited to Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

### FIELD OF THE INVENTION

The present invention is in the field of transactive response DNA binding protein with a molecular weight of 43 kDa (TARDB or also TDP-43). The invention relates to humanized TDP-43 specific binding molecules, in particular to humanized anti-TDP-43 antibodies or an antigen-binding fragment or a derivative thereof and uses thereof. The present invention provides means and methods to diagnose, prevent, alleviate and/or treat a disease, a disorder and/or abnormality associated with TDP-43, in particular associated with TDP-43 aggregates, or TDP-43 proteinopathy, including but not limited to Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

### BACKGROUND

Age-associated brain disorders characterised by pathological aggregation of proteins in the central nervous system (CNS) (proteinopathies) and peripheral organs represent one of the leading causes of disability and mortality in the world. The best characterised protein that forms aggregates is amyloid beta in Alzheimer's disease and related disorders. Other disease-associated, aggregation-prone proteins leading to neurodegeneration include but are not limited to Tau, alpha-synuclein (aSyn, a-syn), huntingtin, fused in sarcoma (FUS), dipeptide repeat proteins (DPRs) produced by unconventional translation of the C9orf72 repeat expansion, superoxide dismutase 1 (SOD1), and TDP-43. Diseases involving TDP-43 aggregates are generally listed as TDP-43 proteinopathies including, but not limited to, ALS and FTD.

### I. TDP-43 Introduction

Transactive response (TAR) DNA binding protein 43 kDa (TDP-43) is a 414-amino acid protein encoded by the TARDBP gene on chromosome 1p36.2 (ALS10). TARDBP is comprised of six exons (exon 1 is non-coding; exons 2-6 are protein-coding). TDP-43 belongs to the family of heterogeneous ribonucleoprotein (hnRNP) RNA binding proteins (Wang et al., Trends in Molecular Medicine Vol. 14 No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology 42 (2010) 1606-1609): two RNA recognition motifs (RRM1 and RRM2), which have two highly conserved hexameric ribonucleoprotein 2 (RNP2) and octameric ribonucleoprotein 1 (RNP1) regions, a nuclear export signal (NES) and a nuclear localization signal (NLS) enabling it to shuttle between the nucleus and the cytoplasm transporting bound mRNA, and a glycine-rich domain at the C-terminal, which mediates protein-protein interactions. TDP-43 is involved in multiple aspects of RNA processing, including transcription, splicing, transport, and stabilization (Buratti and Baralle, FEBS Journal 277 (2010) 2268-2281). It is a highly conserved, ubiquitously expressed protein with a tightly autoregulated expression level that shuttles continuously between the nucleus and cytoplasm, but is predominantly localized to the nucleus. In 2006, TDP-43 was identified as the protein that accumulates in the vast majority of cases of frontotemporal lobar degeneration (FTLD) with tau-negative, ubiquitin-positive inclusions (then referred to as FTLD-TDP), and in most cases of amyotrophic lateral sclerosis (ALS) (Arai et al., Biochemical and Biophysical Research Communications 351 (2006) 602-611; Neumann et al., Science 314, (2006), 130-133).

Thirty-eight negative-dominant mutations in TDP-43 have been identified in sporadic and familial ALS patients as well as in patients with inherited FTD mainly located in the glycine-rich domain (FIG. 1 in Lagier-Tourenne and Cleveland, Cell 136, 2009, 1001-1004). TDP-43 is inherently aggregation-prone, as shown by sedimentation assays, and this propensity is further increased by some of the ALS-associated TARDBP mutations (Ticozzi et al., CNS Neurol. Disord. Drug Targets. 2010, 9(3), 285-296) connecting TDP-43 aggregation with clinical disease manifestation.

### II. TDP-43 in Neurodegeneration

TDP-43 aggregates have been identified in a growing list of neurodegenerative conditions (Lagier-Tourenne et al., Human Molecular Genetics, 2010, Vol. 19, Review Issue 1 R46-R64), including but not limited to: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known as Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's Disease (PD). The term LATE is intended to encompass several previously used designations related to TDP-43 proteinopathy that may be associated with cognitive impairment, including hippocampal sclerosis, hippocampal sclerosis of ageing, hippocampal sclerosis dementia, cerebral age-related TDP-43 with sclerosis (CARTS), and TDP-43 pathologies in the elderly (for reviews see Kuslansky et al., 2004; Lippa and Dickson, 2004; Nelson et al., 2013, 2016b; Dutra et al., 2015).

Aggregated TDP-43 from patient brains shows a number of abnormal modifications, including hyperphosphorylation, ubiquitination, acetylation and C-terminal fragments through proteolytic cleavage (Arai et al., Biochemical and Biophysical Research Communications 351 (2006) 602-611; Neumann et al., Science 314, (2006), 130-133; Neumann et al., Acta Neuropathol. (2009) 117: 137-149; Hasegawa et al., (2008) Annals of Neurology Vol 64 No 1, 60-70; Cohen et al., Nat Commun. 6: 5845, 2015). Another characteristic feature of TDP-43 pathology is redistribution and accumulation of TDP-43 from nucleus to cytoplasm. The hallmark lesions of FTLD-TDP are neuronal and glial cytoplasmic inclusions (NCI and GCI, respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology are described so far by histological classification (Mackenzie and Neumann, J. Neurochem. (2016) 138 (Suppl. 1), 54-70). FTLD-TDP type A cases are characterised by abundant short dystrophic neuritis (DN) and compact oval or crescentic NCI, predominantly in layer II of the neocortex (FIG. 2f in Mackenzie et al., 2016 J. Neurochem. 138 (Suppl. 1), 54-70). Cases with this pathology usually present clinically with either behavioural-variant frontotemporal dementia (bvFTD) or nonfluent/agrammatic variants of Primary Progressive Aphasia (nfvPPA) and are associated with progranulin (GRN) mutations. Type B cases show moderate numbers of compact or granular NCI in both superficial and deep cortical layers with relatively few DN and NII (neuronal intranuclear inclusions; FIG. 2g in Mackenzie et al., 2016 J. Neurochem. 138 (Suppl. 1), 54-70). Most cases with co-appearance of FTD and ALS symptoms are found to have FTLD-TDP type B pathology. Type C cases have an abundance of long, tortuous neurites, predominantly in the superficial cortical laminae, with few or no NCI (FIG. 2j in Mackenzie et al., 2016 J. Neurochem. 138 (Suppl. 1), 54-70). This pathology is particularly found in cases presenting with semantic variant of Primary Progressive Aphasia (svPPA). FTLD-TDP type D displays with abundant lentiform neuronal intranuclear inclusions (NII) and short DN in the neocortex with only rare NCI (FIG. 2k in Mackenzie et al., 2016 J. Neurochem. 138 (Suppl. 1), 54-70). Type E is characterised by granulofilamentous neuronal inclusions (GFNIs) and very fine, dot-like neuropil aggregates affecting all neocortical layers in addition to curvilinear oligodendroglial inclusions in the white matter (Edward B. Lee et al., Acta Neuropathol. 2017 July; 134(1): 65-78). This pattern of pathology is only found in cases with VCP in association with inclusion body myositis.

### III. TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique 169 (2013) 811-819). FTD is presented by several syndromes including bvFTD which is characterised by changes in personality and behaviour; semantic dementia (SD) and progressive nonfluent aphasia (PNFA) characterised by changes in the language function; corticobasal syndrome (CBS), progressive supranuclear palsy syndrome and motor neuron disease (FTD-MND) characterised by movement dysfunction. Clinical diagnosis of these syndromes is complicated and final conclusion can only be achieved through postmortem histopathological analysis to detect aggregated protein and define affected brain regions. In terms of pathological, proteinaceous inclusions, about 45% of cases show pathological accumulation of misfolded Tau, 45% of cases have pathological TDP-43 and a smaller subgroup has aggregates of FUS and other proteins.

### IV. TDP-43 in ALS

Amyotrophic lateral sclerosis (ALS) is a neurodegenerative disorder characterised by the premature loss of upper and lower motor neurons. The progression of ALS is marked by fatal paralysis and respiratory failure with a disease course from diagnosis to death of 1 to 5 years. In most cases of sporadic ALS, the neuropathology is characterised by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of antibodies that specifically bind to phosphorylated TDP-43 in nuclear and cytoplasmic inclusions with amino acids S379, S403, S404, S409, S410 as the major sites of phosphorylation of TDP-43 (Hasegawa et al., Ann Neurol 2008; 64: 60-70; Neumann et al., Acta Neuropathol (2009) 117: 137-149).

V. TDP-43 in AD and other diseases

TDP-43 pathology occurs in up to 57% of brains of patients with Alzheimer's disease (Josephs K A et al., Acta Neuropathol. 2014; 127(6): 811-824; Josephs K A et al., Acta Neuropathol. 2014; 127(3): 441-450; McAleese et al., Brain Pathol. 2017 July; 27(4): 472-479). TDP-43 aggregation is associated with patient's age and correlates with cognitive decline, memory loss and medial temporal atrophy in AD. It appears that in AD, TDP-43 represents a secondary or independent pathology that shares overlapping brain distribution with amyloid beta and tau pathologies in the medial temporal lobe. Pathologic TDP-43 follows a stereotypical pattern of progressive deposition that has been described by the so-called TDP-43 in AD (TAD) staging scheme: TDP-43 first deposits in the amygdala (stage I) followed by hippocampus, limbic, temporal, and finally the frontostriatum (stage V) (Josephs K A et al., Acta Neuropathol. 2014; 127(6): 811-824; Josephs K A et al., Acta Neuropathol. 2014; 127(3): 441-450).

### VI. TDP-43 Spreading

Although ALS and FTD onset and first symptoms vary significantly between patients, the common feature of disease progression is spreading of pathology from an initial focal area to most neurons. The continuous worsening of symptoms might be explained by the progressive spread of TDP-43 pathology. TDP-43 pathology in an ALS patient's brain appears to be spreading in a four-stage process and it is believed that propagation occurs transynaptically via corticofugal axonal projections using anterograde axonal transport (Brettschneider et al., Ann Neurol. 2013 July; 74(1): 20-38). Recent experimental evidence supports the hypothesis of protein propagation in neuronal tissue for amyloid-beta, Tau, alpha-synuclein and TDP-43 by a prion-like mechanism (Hasegawa et al., 2017), with starting points and the topographical spreading patterns being distinct for the four proteins (Brettschneider J et al., Nature Rev. Neuroscience, 2015, 109). The common, disease unifying mechanism is believed to be based on the cell-to-cell spreading of pathological protein aggregates. This mechanism consists of the release of aggregates from a diseased cell, uptake by a naive cell and seeding of the pathological protein conformation by a templated conformational change of endogenous proteins. Pathological TDP-43 able to induce aggregation of physiological (i.e. non-pathological TDP-43) is defined as seeding-competent TDP-43. Indeed, TDP-43 has been found to misfold and aggregate into seeds that are propagating agents with the ability to trigger de novo misfolding. This “prion-like” paradigm is suspected to be one of the key elements in the disease progression. TDP-43 cell-to cell spreading has been studied at a molecular level in few in vitro models, where insoluble TDP-43 preparations from patient brain are able to induce intracellular aggregate formation in receptor cells (Nonaka et al., Cell Reports 4 (2013), 124-134; Feiler et al., 2015; Porta et al., Nat. Comm., 2018). Further, it has been observed that intracellular TDP-43 aggregates are released in association with exosome prior to spreading to the next cell (Nonaka et al., Cell Reports 4 (2013, 124-134)). Similarly, adenovirus-transduced TDP-43 expression led to cytoplasmic aggregates which were phosphorylated, ubiquitinated and more importantly acted as seeds initiating cell-to-cell spreading (Ishii et al., PLoS ONE 12(6): e0179375, 2017). The patient-derived pathological TDP-43 can lead to widespread deposition of endogenous TDP-43 following intracerebral inoculation into transgenic and wildtype mice (Porta et al., Nat. Comm., 2018).

### VII. Prevention and Treatment of TDP-43 Proteinopathies

TDP-43 aggregation and spreading of pathology are major hallmarks of ALS and FTD—fatal diseases for which currently no cure is available. Therefore, there is a need for new methods for the treatment and prevention of TDP-43 proteinopathies. Mutations in TDP-43 are associated with familial cases of ALS and FTD providing causative link between TDP-43 misfolding and disease progression.

### VIII. Diagnosis of TDP-43 Proteinopathies

The diagnosis of FTD based on clinical manifestations is insufficient since the clinical representation can overlap with other diseases, in particular in the earlier stages. A number of approaches aims at the development of biochemical biomarkers to distinguish different types of FTD pathology. Development of antibodies against different conformations of TDP-43 may permit generating more sensitive and specific diagnostic tools. In parallel to biochemical biomarkers, the development of imaging biomarkers may enable early and specific detection of the pathology in TDP-43 proteinopathies. The ability to image TDP-43 deposition in the brain may be a substantial achievement for diagnosis and drug development for TDP-43 proteinopathies. Using cell permeable antibody fragments could enable such detection. The earliest event in neurodegenerative diseases based on misfolding of different proteins is the acquisition of an alternative conformation that renders the protein toxic. Moreover, this misfolded conformation can self-propagate by recruiting the endogenous, normal protein into the misfolded conformation as mechanistic basis for the observed spread through affected tissue. To develop antibodies against different conformational states of a given protein, supramolecular antigenic constructs were designed in which the conformation of the presented antigen was controlled to raise conformational-specific antibodies against a given target in a specific conformational state (WO2012/055933 and WO2012/020124). Conformational-specific antibodies offer many advantages since they can discriminate between the disease-associated and the functional, endogenous conformation of these proteins. This approach offers many advantages in the therapeutic application since such antibodies are less likely to be adsorbed by the normal conformation of proteins while targeting the misfolded, disease associated isoform thereof. Similar to this, for diagnostic application such antibodies only recognize the disease-associated, structural state of a protein, which is paramount for the development of the sensitive and specific diagnostics.

The use of a TDP-43-based biomarker in TDP-43 proteinopathies still remains to be established. Such evaluation has been hindered in part due to the lack of high affinity antibodies that can be employed in a suitable immunoassay for quantification of pathological TDP-43 in biofluids (Feneberg et al., Molecular Neurobiology, 2018).

Therefore, there is a clear need for biomarkers able to detect misfolded aggregated TDP-43 and non-aggregated physiological TDP-43, in particular in a human sample, for diagnosing different types of TDP-43 proteinopathies and/or for monitoring efficacy of therapeutic drugs used for treatment of diseases, disorders and abnormalities associated with TDP-43, in particular associated with TDP-43 aggregates or TDP-43 proteinopathy.

The TDP-43 proteinopathies are defined as a set of neurodegenerative disorders characterised by pathological TDP-43.

### IX. Prior Art

Patent application WO2008/151055 discloses methods and materials for using the levels of TDP-43 polypeptides and/or TDP-43 polypeptide cleavage products (e.g. 25 kD and 35 kD TDP-43 polypeptide cleavage products) in a biological fluid to determine whether or not a mammal has a neurodegenerative disease.

Patent application WO2013/061163 discloses TDP-43 specific binding molecules including polypeptides such as human antibodies as well as fragments, derivatives and variants thereof. Patent application WO2020/234473 discloses specific binding molecules including polypeptides such as murine antibodies or antigen-binding fragments thereof.

The dosing regimen is a critical element of the drug's target product profile. Due to the suboptimal brain penetration of biologics, currently the drug exposure is reached only following repeated administration of the antibody. To ensure an efficient drug distribution required for the desired pharmacological effect of an antibody, a suitable pharmacokinetic profile is essential for its in vivo therapeutic applications. For some antibodies, optimization of pharmacokinetic parameters such as clearance and/or volume of distribution can maximize drug exposure in vivo. Antibody are recycled through the interaction of their Fc domain with the neonatal Fc receptor (FcRn). Increased affinity to FcRn can be achieved by protein engineering and translates in an improved recycling profile and a longer antibody half-life. Several sets of mutations have been described such as the YTE (Dall' Acqua et al. journal of immunology, 2002, 69:5171-5180) or the LS (Zalevsky, J et al, Nat Biotechnol 28(2):157-9, 2010) mutation which led to an improved pharmacokinetic profile in vivo. In some cases, high pI and patches of positive charges have been shown to negatively impact antibody clearance (Igawa et al, PEDS. 2010; vol. 23 no. 5 pp. 385-392, Bumbaca et al, JBC, VOL. 290, NO. 50, pp. 29732-29741, 2015).

### SUMMARY

As currently there are no TDP-43 antibody therapies on the market, there is a need for anti-TDP-43 antibodies with suitable pharmacokinetic parameters for in vivo therapeutic applications. Without wishing to be bound by any particular theory, it is believed that the binding molecules of the invention, in particular humanized antibodies or antigen-binding fragments thereof, comprise humanized acceptor frameworks which result in optimal half-lives for in vivo therapeutic applications. The binding molecules of the invention (especially hACI-7069-633B12-Ab1_H33L27 and hACI-7069-633B12-Ab1_H32L27) are shown herein to provide beneficial pharmacokinetic and pharmacodynamic properties. See Examples 5-14 and Tables 9, 10 and 11 herein. The binding molecules of the invention display suitable half-life and clearance values for therapeutic use.

There is a need for humanized anti TDP-43 binding molecules which bind misfolded aggregated TDP-43 and non-aggregated physiological TDP-43 with suitable pharmacokinetic parameters for in vivo therapeutic applications. Such humanized binding molecules, in particular antibodies and antigen-binding fragments thereof may bind to a specific epitope of human TDP-43 (SEQ ID NO: 1). Moreover, the development of sensitive and specific biomarkers allowing the differentiation between types of pathology within the FTD spectrum is an urgent task.

The technical problem is solved by the embodiments provided herein.

The binding molecules of the invention are humanized forms of a murine antibody, in particular, they are humanized forms of a murine monoclonal antibody that binds to human TDP-43 (SEQ ID NO: 1). The antibody referred to herein as ACI-7069-633B12-Ab1 was selected for the development of humanized binding molecules. This antibody derives from hybridoma clone 633B12C8, as described herein. It binds to an epitope within amino acids 397-411 of human TDP-43 (SEQ ID NO: 1). More specifically it binds to an epitope from amino acids 400-405 of human TDP-43 (SEQ ID NO: 1). This antibody has (is encoded by) a VH nucleotide sequence as set forth in SEQ ID NO: 28 and a VL nucleotide sequence as set forth in SEQ ID NO: 29, see Table 10 of WO2020/234473. This antibody has a VH amino acid sequence as set forth in SEQ ID NO: 20 and a VL amino acid sequence as set forth in SEQ ID NO: 24, see Table 11 of WO2020/234473. This antibody has a VH CDR1 amino acid sequence as set forth in SEQ ID NO: 21, a VH CDR2 amino acid sequence as set forth in SEQ ID NO: 22 and a VH CDR3 amino acid sequence of ES, see Table 11 of WO2020/234473. This antibody has a VL CDR1 amino acid sequence as set forth in SEQ ID NO: 25, a VL CDR2 amino acid sequence as set forth in SEQ ID NO: 16 and a VL CDR3 amino acid sequence as set forth in SEQ ID NO: 27, see Table 11 of WO2020/234473.

The chosen CDR sequences may be mutated at specific positions. In some embodiments such mutations are made to avoid potential post-translational modification sites. In specific embodiments, one or more, up to all of the following residues are mutated in the VH region (CDRH2): N53, N54 and G55. Specific mutations include N53G, N53S, N53Q, N54Q, N54G and G55A. Residues are numbered according to Kabat. In specific embodiments, one or more, up to all of the following residues are mutated in the VL region (CDRL1 and/or CDRL2 and/or CDRL3): K24, D28, G29, D55, S56 and W89. Residues are numbered according to Kabat. Specific mutations include K24R, D28E, D28G, G29A, D55E, S56A, W89Y, W89F and W89L. In specific embodiments, the humanized binding molecules, in particular humanized antibodies or antigen-binding fragments thereof of the invention comprise a human heavy chain variable domain subfamily 1 framework sequence. More specifically, the humanized binding molecules, in particular humanized antibodies or antigen-binding fragments thereof of the invention may comprise an IGHV1-69-2 (IMGT accession number KF698734, Z29977, Z12305; UniProtKB AOAOG2JMI3), IGHV1-3 (IMGT accession numbers X62109, X62107, MK540645, MH779622 and MN337616; UniProtKB-AOAOC4DH29), IGHV1-2 (IMGT accession numbers X07448, X62106, X92208, KF698733, HM855674, MH267285 and MN337615; UniProtKB-P23083), IGHV1-46 (IMGT accession numbers X92343, J00240, L06612 and MK540650; UniProtKB-P01743), IGHV1-24 (IMGT accession number M99642; UniProtKB-AOAOC4DH33), IGHV5-51 (IMGT accession number M99686, M18806, X56368, X56367, Z27449, MK321694, IMGT000055; UniProtKB AOAOC4DH38) or IGHV3-43 (IMGT accession number M99672, HM855392; UniProtKB AOAOB4J1X8) VH framework sequence, preferably a IGHV1-69-2 VH framework sequence.

In specific embodiments, the humanized binding molecules, in particular humanized antibodies or antigen-binding fragments thereof of the invention comprise a human light chain variable domain kappa subfamily 2 framework sequence. More specifically, the humanized binding molecules, in particular humanized antibodies or antigen-binding fragments thereof of the invention may comprise an IGKV2-30 (IMGT accession numbers X63403 and FM164408; UniProtKB-P06310), IGKV2-29 (IMGT accession numbers X63396, U41645 and AJ783437; UniProtKB-A2NJV5), IGKV2D-29 (IMGT accession numbers M31952 and U41644; UniProtKB-A0A075B6S2) IGKV2-28 (IMGT accession numbers X63397; UniProtKB-A0A075B6P5), IGKV2-24 (IMGT accession number X12684; UniProtKB-AOAOC4DH68), IGKV2D-28 (IMGT accession numbers X12691; UniProtKB-P01615), IGKV2-40 (IMGT accession numbers X59314 and X59317; UniProtKB-A0A087WW87), IGKV2D-40 (IMGT accession numbers X59311; UniProtKB-P01614) or IGKV4-1 (IMGT accession numbers Z00023 and MW316673; UniProtKB-P06312) VL framework sequence, preferably a IGKV2-40, IGKV2D-40, IGKV2D-28, IGKV4-1 or IGKV2-28 VL framework sequence, most preferably a IGKV2-28 (IMGT accession numbers X63397; UniProtKB-A0A075B6P5) VL framework sequence.

In one embodiment, the humanized binding molecules of the invention, in particular humanized antibodies or antigen-binding fragments thereof of the invention comprise IGHV1-69-2 and IGKV2-28 framework sequences.

The chosen framework sequences may be mutated at specific positions. In some embodiments such mutations are made to positively influence CDR loop conformation and/or variable domain packing between VH and VL domains. In certain embodiments, one or more, up to all of the residues listed in Table 2 below are mutated, according to Kabat numbering. In specific embodiments, one or more, up to all of the following VH mutations are made, according to Kabat numbering: V24T, Y27F, M48I, Q64K, A71V, T73K, T94R. In specific embodiments, one or more, up to all of the following VL mutations are made (according to Kabat numbering): I2V, Y36L, Q45K, L46R, G57R. In specific embodiments, one or more, up to all of the following VH mutations are made: V24T, Y27F, M48I, Q64K, A71V, T73K, T94R and one or more, up to all of the following VL mutations are made: I2V, Y36L, Q45K, L46R, G57R, all according to Kabat numbering.

Accordingly, the invention relates to humanized binding molecules, in particular humanized antibodies or antigen-binding fragments thereof, which specifically recognize misfolded aggregated TDP-43 and non-aggregated physiological TDP-43. Within the invention, misfolded TDP-43 includes misfolded monomeric and/or misfolded oligomeric and/or misfolded aggregated and/or post-translationally modified and/or misfolded truncated TDP-43. Post-translationally modified TDP-43 comprises phosphorylated, ubiquitylated, acetylated, sumoylated, and/or methylated TDP-43. Physiological TDP-43 includes soluble nuclear TDP-43. It is demonstrated herein that the humanized binding molecules of the invention are capable of binding pathological TDP-43, including TDP-43 aggregates and phosphorylated TDP-43. Thus, the invention provides humanized binding molecules, in particular humanized antibodies or antigen-binding fragments thereof, which specifically recognize misfolded aggregated TDP-43 and non-aggregated physiological TDP-43. Such binding molecules are referred to herein as humanized “pan-TDP-43” binding molecules, in particular humanized pan-TDP-43 antibodies. As explained herein, the humanized TDP-43 binding molecules of the invention may bind misfolded aggregated TDP-43 and non-aggregated physiological TDP-43 equally, or to one preferentially to the other whilst binding to both categories of TDP-43 specifically. The invention also provides the humanized binding molecules, in particular humanized antibodies or antigen-binding fragments thereof, for the prevention, alleviation, treatment and/or diagnosis of diseases, disorders and abnormalities associated with TDP-43, in particular associated with TDP-43 aggregates, or TDP-43 proteinopathy. The invention also provides the humanized binding molecules, in particular humanized antibodies or antigen-binding fragments thereof, for detecting and/or understanding (i.e. identifying) the specific type of pathology causing neurodegeneration. Envisaged are uses as diagnostic biomarkers enabling more efficient and precise subject selection for longitudinal monitoring in clinical studies, supporting the development of novel therapeutics for TDP-43 proteinopathies.

The invention also provides the humanized TDP-43 binding molecules, in particular humanized antibodies or antigen-binding fragments thereof, as a medicine (therapeutic agent).

Without wishing to be bound by theory, the present invention was developed based on the assumption that modified conformation-specific antigenic peptides and peptide fragments derived from TDP-43 protein or the whole TDP-43 protein and the humanized antibodies obtainable or obtained by using said peptides or fragments or the whole TDP-43 protein as antigen block TDP-43 cell-to-cell propagation, and/or disaggregate TDP-43 aggregates and/or block TDP-43 seeding and/or neutralize seeding-competent TDP-43 and/or inhibit the aggregation of TDP-43 protein or fragments thereof and/or potentiate TDP-43 clearance. The humanized binding molecules of the invention, in particular humanized polypeptides, more particularly humanized antibodies or antigen-binding fragments thereof, bind to misfolded aggregated TDP-43, particularly to cytoplasmic and extracellular misfolded TDP-43. The humanized binding molecules of the invention, in particular humanized polypeptides, more particularly humanized antibodies or antigen-binding fragments thereof, bind to full-length TDP-43 and/or truncated TDP-43. In one embodiment, the humanized binding molecules of the invention, in particular humanized polypeptides, more particularly humanized antibodies or antigen-binding fragments thereof, specifically bind to cytoplasmic misfolded TDP-43. In one embodiment, the humanized TDP-43 binding molecules of the invention, in particular humanized antibodies or antigen-binding fragments thereof, bind and neutralize seeding-competent TDP-43. In one embodiment, the humanized TDP-43 binding molecules of the invention, in particular humanized antibodies or antigen-binding fragments thereof, bind extracellular and/or aggregated TDP-43 and potentiates its clearance by immune cells such as microglia through antibody-dependent cellular phagocytosis (ADCP).

Misfolded aggregated, or pathology-associated, TDP-43 is composed of TDP-43 proteins that lose their normal folding (i.e. are misfolded) and localization. Misfolded aggregated TDP-43 can be found in preinclusions and in neuronal and glial cytoplasmic inclusions (NCI and GCI, respectively), neuronal intranuclear inclusions (NII) and dystrophic neurites (DN) that are immunoreactive for TDP-43.

Non-aggregated physiological TDP-43 is physiologically functional TDP-43 protein predominantly located in the nucleus and shuttling to the cytoplasm, being in a status able to exhibit its desired function in an in vivo cellular environment.

The humanized TDP-43 binding molecules of the invention, in particular the humanized anti-TDP-43 antibodies or antigen-binding fragments thereof, surprisingly have at least one, preferably two, more preferably three, even more preferably four, still more preferably five, still even more preferably six, most preferably all seven of the following characteristics:
blocks TDP-43 cell-to-cell propagation;
disaggregates TDP-43 aggregates;
inhibits the aggregation of TDP-43 protein or fragments thereof;
blocks TDP-43 seeding;
neutralizes seeding-competent TDP-43;
blocks TDP-43 spreading; and
potentiates TDP-43 clearance.

Independent of the combination of one, two, three, four, five, six or seven above listed characteristics, the humanized anti-TDP-43 binding molecules, preferably humanized anti-TDP-43 antibodies or antigen-binding fragments thereof, of the invention may ameliorate/inhibit/reduce the formation of TDP-43 pathology in an in vivo model of TDP-43 proteinopathies and more importantly in patients with TDP-43 pathology.

The humanized anti-TDP-43 binding molecules bind to a region within amino acids 397-411 of human TDP-43 (SEQ ID NO: 1), more specifically the humanized TDP-43 binding molecules bind to an epitope within amino acids residues 400-405 of human TDP-43 (SEQ ID NO: 1). According to the invention there is provided a humanized TDP-43 binding molecule, in particular a humanized TDP-43 antibody or an antigen-binding fragment thereof, comprising:
a. a VH-CDR1 comprising the amino acid sequence of SEQ ID NO: 21;
b. a VH-CDR2 selected from the amino acid sequence of SEQ ID NO: 22, SEQ ID NO: 82, SEQ ID NO: 92; SEQ ID NO: 102, SEQ ID NO: 112 or SEQ ID NO: 122;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16;
f. a VL-CDR3 selected from the amino acid sequence of SEQ ID NO: 27 or SEQ ID NO:87; and
g. an acceptor framework of a Heavy Chain Variable domain (VH) selected from the group consisting of IGHV1-69-2, IGHV5-51 or IGHV3-43, preferably IGHV1-69-2; and/or
h. an acceptor framework of a Light Chain Variable domain (VL) selected from the group consisting of IGKV2-28, IGKV2-24, IGKV2D-28, IGKV2-40, IGKV2D-40 or IGKV4-1, preferably IGKV2-28.

In preferred embodiments, the humanized TDP-43 binding molecules comprise an acceptor framework of the VH comprising one or more and preferably all of the following VH mutations:
a. V24T;
b. M48I;
c. A71V;
d. T73K; and
e. T94R; and/or comprise an acceptor framework of the VL comprising one or more the following VL mutations:
f. I2V;
g. Y36L;
h. Q45K;
i. L46R; and
j. G57R (numbering is according to Kabat).

In preferred embodiments, the humanized TDP-43 binding molecules comprise an acceptor framework of the VH comprising one or more and preferably all of the following VH mutations:
a. V24T;
b. Y27F
c. M48I;
d. A71V;
e. T73K; and
f. T94R; and/or comprise an acceptor framework of the VL comprising one or more the following VL mutations:
g. I2V;
h. Y36L;
i. Q45K;
j. L46R; and
k. G57R (numbering is according to Kabat).

In more preferred embodiments, the humanized TDP-43 binding molecules comprise an acceptor framework of the VH comprising all of the following VH mutations:
a. V24T;
b. M48I;
c. A71V;
d. T73K; and
e. T94R; and/or comprising an acceptor framework of the VL comprising one or more, preferably all of the following VL mutations:
f. Y36L;
g. L46R; and
h. G57R (numbering is according to Kabat).

In more preferred embodiments, the humanized TDP-43 binding molecules comprise an acceptor framework of the VH comprising all of the following VH mutations:
a. V24T;
b. Y27F
c. M48I;
d. A71V;
e. T73K; and
f. T94R; and/or comprising an acceptor framework of the VL comprising one or more, preferably all of the following VL mutations:
i. Y36L;
j. L46R; and
k. G57R (numbering is according to Kabat).

In a preferred embodiment, the humanized TDP-43 binding molecules comprise an acceptor framework of the VH comprising all of the following VH mutations:
a. V24T;
b. M48I;
c. A71V;
d. T73K; and
e. T94R; and comprising an acceptor framework of the VL comprising one or more, preferably all of the following VL mutations:
f. Y36L;
g. L46R; and
h. G57R (numbering is according to Kabat).

In a preferred embodiment, the humanized TDP-43 binding molecules comprise an acceptor framework of the VH comprising all of the following VH mutations:
a. V24T;
b. Y27F
c. M48I;
d. A71V;
e. T73K; and
f. T94R; and comprising an acceptor framework of the VL comprising one or more, preferably all of the following VL mutations:
i. Y36L;
j. L46R; and
k. G57R (numbering is according to Kabat).

The humanized TDP-43 binding molecule, in particular a humanized TDP-43 antibody or an antigen-binding fragment thereof, may comprise:
a. a VH-CDR1 comprising the amino acid sequence of SEQ ID NO: 21;
b. a VH-CDR2 selected from the amino acid sequence of SEQ ID NO: 112, SEQ ID NO: 122 or SEQ ID NO: 102;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16; and
f. a VL-CDR3 selected from the amino acid sequence of SEQ ID NO: 27 or SEQ ID NO:87.

In preferred embodiments, the humanized TDP-43 binding molecule, in particular the humanized TDP-43 antibody or antigen-binding fragment thereof, comprises:
a. a VH-CDR1 comprising the amino acid sequence of SEQ ID NO: 21;
b. a VH-CDR2 selected from the amino acid sequence of SEQ ID NO: 112 or SEQ ID NO: 122;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16; and
f. a VL-CDR3 selected from the amino acid sequence of SEQ ID NO: 27 or SEQ ID NO:87.

In some embodiments, the humanized TDP-43 binding molecules of the invention comprise:
VH-CDR1 comprising the amino acid sequence of SEQ ID NO: 21; VH-CDR2 comprising the amino acid sequence of SEQ ID NO: 22; and VH-CDR3 comprising the amino acid sequence ES (Glu-Ser); and VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 27; or
VH-CDR1 comprising the amino acid sequence of SEQ ID NO: 21; VH-CDR2 comprising the amino acid sequence of SEQ ID NO: 112; and VH-CDR3 comprising the amino acid sequence ES (Glu-Ser); and VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 27;
VH-CDR1 comprising the amino acid sequence of SEQ ID NO: 21; VH-CDR2 comprising the amino acid sequence of SEQ ID NO: 112; and VH-CDR3 comprising the amino acid sequence ES (Glu-Ser); and VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87;
VH-CDR1 comprising the amino acid sequence of SEQ ID NO: 21; VH-CDR2 comprising the amino acid sequence of SEQ ID NO: 122; and VH-CDR3 comprising the amino acid sequence ES (Glu-Ser); and VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 27;
VH-CDR1 comprising the amino acid sequence of SEQ ID NO: 21; VH-CDR2 comprising the amino acid sequence of SEQ ID NO: 122; and VH-CDR3 comprising the amino acid sequence ES (Glu-Ser); and VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87;
VH-CDR1 comprising the amino acid sequence of SEQ ID NO: 21; VH-CDR2 comprising the amino acid sequence of SEQ ID NO: 112; and VH-CDR3 comprising the amino acid sequence ES (Glu-Ser); and VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 87;
VH-CDR1 comprising the amino acid sequence of SEQ ID NO: 21; VH-CDR2 comprising the amino acid sequence of SEQ ID NO: 122; and VH-CDR3 comprising the amino acid sequence ES (Glu-Ser); and VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 87;

The invention is further directed, inter alia, to (i) an immunoconjugate comprising the humanized TDP-43 binding molecule, (ii) a labeled antibody comprising the humanized TDP-43 binding molecule, (iii) a pharmaceutical composition comprising the humanized TDP-43 binding molecule and a pharmaceutically acceptable carrier and/or excipients and/or diluents, (iv) a humanized TDP-43 binding molecule for human or veterinary medicine use, (v) a humanized TDP-43 binding molecule for use in the prevention, alleviation, treatment of diseases, disorders and/or abnormalities associated with TDP-43 or TDP-43 proteinopathy, (vi) a humanized TDP-43 binding molecule for diagnostic use (in particular for in vivo diagnosis, but also for in vitro testing), (vii) a humanized TDP-43 binding molecule for research use, in particular as an analytical tool or reference molecule, (viii) a humanized TDP-43 binding molecule for use as a diagnostic tool to monitor diseases, disorders and/or abnormalities associated with TDP-43 43 or TDP-43 proteinopathy, (ix) a method of retaining or increasing cognitive memory capacity or slowing memory loss in an individual with a disease, disorder and/or abnormality associated with TDP-43 or a TDP-43 proteinopathy by treating the individual with a humanized TDP-43 binding molecule, (x) a method of reducing the level of aggregated TDP-43 and/or phosphorylated TDP-43 in an individual by treating the individual with a humanized TDP-43 binding molecule, (xi) a nucleic acid molecule encoding the humanized TDP-43 binding molecule, (xii) a recombinant expression vector comprising a nucleic acid molecule of the present invention, (xiii) a host cell comprising the nucleic acid and/or the vector of present invention, (xiv) a cell-free expression system containing the recombinant expression vector of present invention, (xv) a method for producing a humanized TDP-43 binding molecule, (xvi) a method of quantifying TDP-43 in a sample obtained from a subject using a humanized TDP-43 binding molecule, and (xvii) a kit comprising humanized TDP-43 binding molecules of the invention and/or nucleic acids, expression vectors, host cells and/or cell free expression systems for producing the same.

The humanized TDP-43 binding molecules of the invention, in particular the humanized anti TDP-43 antibodies or antigen-binding fragments thereof, may recruit and/or activate microglia. More specifically, the humanized TDP-43 binding molecules of the invention may affect microglial morphology in terms of cell size and activation state. This may contribute to the reduction of TDP-43 pathology demonstrated by the TDP-43 binding molecules of the invention.

In the present invention, the humanized binding molecules, in particular humanized antibodies or antigen-binding fragments thereof, specifically recognize TDP-43. The humanized binding molecules of the invention include humanized polypeptides and/or humanized antibodies and/or antigen-binding fragments thereof specific to/for the TDP-43 protein. “Specifically recognize TDP-43” means that the humanized binding molecules of the invention specifically, generally, and collectively, bind to TDP-43, in particular some epitopes within TDP-43, in particular an epitope exposed/accessible in one or more pathological conformation(s) of TDP-43 protein, with greater affinity than for other epitopes. The humanized binding molecules of the invention, in particular humanized polypeptides, more particularly humanized antibodies or antigen-binding fragments thereof, that specifically bind to TDP-43, specifically recognize misfolded aggregated TDP-43 and non-aggregated physiological TDP-43.

The humanized TDP-43 binding molecules of the invention, in particular the humanized antibodies or antigen-binding fragments thereof, bind to both non-aggregated physiological TDP-43 and aggregated TDP-43. Thus, humanized TDP-43 binding molecules of the invention, in particular the humanized antibodies or antigen-binding fragments thereof, may bind approximately equally well to soluble and aggregated TDP-43. Humanized TDP-43 binding molecules of the invention, in particular the humanized antibodies or antigen-binding fragments thereof, may bind approximately equally to aggregated TDP-43 as compared with non-aggregated TDP-43. More particularly, humanized TDP-43 binding molecules of the invention, in particular the humanized antibodies or antigen-binding fragments thereof, may bind approximately equally to aggregated TDP-43 in the cytoplasm as compared with non-aggregated TDP-43 in the nucleus. In other embodiments, humanized TDP-43 binding molecules of the invention, in particular the humanized antibodies or antigen-binding fragments thereof, may preferentially bind to aggregated TDP-43 as compared with non-aggregated TDP-43, whilst binding to both species. More particularly, humanized TDP-43 binding molecules of the invention, in particular the humanized antibodies or antigen-binding fragments thereof, may preferentially bind to aggregated TDP-43 in the cytoplasm as compared with non-aggregated TDP-43 in the nucleus, whilst binding to both species. Alternatively, in other embodiments, humanized TDP-43 binding molecules of the invention, in particular the humanized antibodies or antigen-binding fragments thereof, may preferentially bind to non-aggregated TDP-43 as compared with aggregated TDP-43, whilst binding to both species. More particularly, humanized TDP-43 binding molecules of the invention, in particular the humanized antibodies or antigen-binding fragments thereof, may preferentially bind to non-aggregated TDP-43 in the nucleus as compared with aggregated TDP-43 in the cytoplasm, whilst binding to both species. These binding properties may be demonstrated for example using immunohistochemistry.

In some embodiments, the invention encompasses humanized binding molecules, particularly humanized antibodies and antigen-binding fragments thereof of the invention as described herein that specifically bind TDP-43 and the use of these humanized binding molecules to diagnose, prevent, alleviate and/or treat a disease, disorder and/or abnormality associated with TDP-43, in particular associated with TDP-43 aggregates, or TDP-43 proteinopathy including, but not limited to, frontotemporal dementia (FTD), amyotrophic lateral sclerosis (ALS), Alzheimer's disease (AD), Parkinson's disease (PD), Chronic Traumatic Encephalopathy (CTE) and limbic-predominant age-related TDP-43 encephalopathy (LATE). The methods and compositions disclosed herein have applications in diagnosing, preventing, alleviating and/or treating a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS). Preferably, the use of these humanized binding molecules to diagnose, prevent, alleviate and/or treat a disease, disorder and/or abnormality associated with TDP-43, in particular associated with TDP-43 aggregates, or TDP-43 proteinopathy is directed to amyotrophic lateral sclerosis (ALS), Alzheimer's disease (AD) or Frontotemporal dementia (FTD). More preferably, the use is directed to amyotrophic lateral sclerosis (ALS). More preferably, the use is directed to Alzheimer's disease (AD). More preferably, the use is directed to Frontotemporal dementia (FTD).

In another embodiment, a humanized TDP-43 binding molecule, particularly a humanized anti TDP-43 antibody or an antigen-binding fragment thereof of the invention as described herein specific for TDP-43 is contacted with a sample to detect, diagnose and/or monitor a disease, disorder and/or abnormality associated with TDP-43, in particular associated with TDP-43 aggregates, or TDP-43 proteinopathy, selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In one embodiment, the invention encompasses humanized binding molecules, particularly humanized antibodies or antigen-binding fragments thereof of the invention as described herein that specifically bind TDP-43 and the use of these humanized binding molecules, particularly of these humanized antibodies, to detect the presence of TDP-43 in a sample. Accordingly, humanized TDP-43 binding molecules of the invention, such as, humanized anti-TDP43 antibodies as described herein, can be used, inter alia, to screen a clinical sample, in particular human blood, cerebrospinal fluid (CSF), interstitial fluid (ISF) and/or urine for the presence of TDP-43 in samples, for example, by using an ELISA-based or surface adapted assay. Tissue samples may be used in some circumstances, such as brain tissue samples. The methods and compositions of the invention also have applications in diagnosing presymptomatic disease and/or in monitoring disease progression and/or therapeutic efficacy. According to some embodiments, a humanized antibody specific for TDP-43 (e.g., a full-length humanized antibody or a TDP-43 binding fragment or derivative of a humanized antibody) is contacted with a sample (e.g., blood, urine, cerebrospinal fluid (CSF), interstitial fluid (ISF) or brain tissue) to detect, diagnose and/or monitor Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD). The humanized TDP-43 binding molecules of the invention may be used to quantify TDP-43 in suitable samples, in particular clinical samples such as blood, brain tissue, CSF, ISF or urine, with relatively high TDP-43 levels, as compared to a suitable control, indicating disease and/or more advanced disease. Many suitable immunoassay formats are known. Thus, the methods (such as ELISA, MSD (Meso Scale Discovery), HTRF (Homogeneous Time Resolved Fluorescence) and AlphaLISA) may be performed for diagnostic purposes with high levels of TDP-43 indicating disease. Alternatively, the methods may be performed for monitoring purposes. Increased levels over time may indicate progression of the disease. Decreased levels over time may indicate regression of the disease. The methods may also be used to monitor therapy, in particular to monitor the efficacy of a particular treatment. Successful therapy may be measured with reference to stable or decreasing levels of TDP-43 following treatment. It is demonstrated in Example 12 of WO2020/234473 that TDP-43 levels were higher in CSF samples from TDP-43 proteinopathy patients than in control samples taken from healthy subjects (healthy control). The control samples may or may not be run in parallel with the test samples. In some embodiments control levels are determined from a series of control samples taken from healthy subjects under similar or the same experimental conditions and used as a comparator for levels determined in the test sample. Methods of quantifying TDP-43 in suitable samples using humanized binding molecules of the invention may also be used to select a therapy (for further treatment of the subject). Thus, personalized treatment methods are envisaged. A sample is taken before and after treatment. If treatment using the therapy results in stable or, preferably, decreasing levels of TDP-43 following treatment the therapy may be selected for that subject. If the therapy does not result in stable or, preferably, decreasing levels of TDP-43 following treatment the therapy is not selected for the subject. The therapy may be any suitable candidate therapeutic agent for treatment of TDP-43 proteinopathies. In preferred embodiments, the therapy comprises a humanized TDP-43 binding molecule of the invention, typically in the form of a pharmaceutical composition as described herein.

The humanized TDP-43 binding molecules of the invention may also be used for disease classification into particular types or subtypes. Thus, there is provided a method for classifying a disease, disorder and/or abnormality associated with TDP-43, in particular associated with TDP-43 aggregates or for classifying a TDP-43 proteinopathy comprising:
a. performing the methods of the invention in which levels of TDP-43 are quantified, as compared to suitable controls,
b. optionally identifying mutations in a sample from the subject including but not limited to progranulin (GRN) mutation, C9orf72 mutations, TARDBP mutation, angiogenin (ANG) mutation, mutation in the valosin-containing protein (VCP), mutation in the myotilin (MYOT) gene or mutations in the gene coding for desmin (DES), and
c. classifying the disease, disorder and/or abnormality associated with TDP-43, in particular associated with TDP-43 aggregates, or TDP-43 proteinopathy.

Similarly, there is provided a method for classifying a disease, disorder and/or abnormality associated with TDP-43, in particular associated with TDP-43 aggregates, or for classifying a TDP-43 proteinopathy comprising: performing the methods of the invention in which levels of TDP-43 are quantified in a sample obtained from a subject with a disease, disorder and/or abnormality associated with TDP-43, or TDP-43 proteinopathy, wherein the levels are compared with control samples taken from subjects with different types or subtypes of disease, disorder and/or abnormality (i.e. a representative set of control levels are determined for the types or subtypes of interest) associated with TDP-43, in particular associated with TDP-43 aggregates, or TDP-43 proteinopathy; and classifying the disease, disorder and/or abnormality associated with TDP-43, in particular associated with TDP-43 aggregates, or TDP-43 proteinopathy based on the comparison. Thus, the classification is based on determining the closest match between the test sample and one or more of the control samples. These methods may further comprise identifying mutations in the sample including but not limited to progranulin (GRN) mutation, C9orf72 mutations, TARDBP mutation, angiogenin (ANG) mutation), mutation in the valosin-containing protein (VCP), mutation in the myotilin (MYOT) gene or mutations in the gene coding for desmin (DES), wherein the identified mutations are also used to classify the disease, disorder and/or abnormality associated with TDP-43, in particular associated with TDP-43 aggregates, or TDP-43 proteinopathy. For the avoidance of doubt, the identification of mutations in a sample may be performed by any suitable method; for example, based on nucleic acid sequencing of nucleic acid molecules within the sample. The sample may be separate and distinct from the sample in which TDP-43 levels are determined, but is from the same subject.

In other embodiments, the invention provides methods for preventing, alleviating and/or treating a disease, disorder and/or abnormality associated with TDP-43, in particular associated with TDP-43 aggregates, or TDP-43 proteinopathy. According to one embodiment, the methods of the invention comprise administering an effective concentration of a humanized binding molecule, particularly a humanized antibody of the invention specific for TDP-43 (e.g., a full-length antibody or a TDP-43 binding fragment or derivative of an antibody) as described herein to a subject. In another embodiment, the invention provides a method for preventing, alleviating and/or treating a TDP-43 proteinopathy. According to some embodiments, a humanized binding molecule, particularly a humanized antibody of the invention or an antigen-binding fragment thereof as described herein specific for TDP-43 is administered to treat, alleviate and/or prevent frontotemporal degeneration (FTD) or amyotrophic lateral sclerosis (ALS). In another embodiment, a humanized binding molecule, particularly a humanized antibody of the invention or an antigen-binding fragment thereof as described herein specific for TDP-43 is administered to prevent, alleviate and/or treat a neurodegenerative disease selected from frontotemporal dementia (FTD), amyotrophic lateral sclerosis (ALS), Alzheimer's disease (AD, including sporadic and familial forms of AD), Parkinson's disease (PD), Chronic Traumatic Encephalopathy (CTE), limbic-predominant age-related TDP-43 encephalopathy (LATE).

In another embodiment, a humanized binding molecule, particularly a humanized antibody of the invention or antigen-binding fragment thereof as described herein specific for TDP-43 is administered to prevent, alleviate and/or treat a disease selected from: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

### DETAILED DESCRIPTION OF EMBODIMENTS OF THE INVENTION

### X. Definitions

An “antigen binding molecule,” as used herein, is any molecule that can specifically or selectively bind to an antigen, in particular TDP-43. A binding molecule may include or be an antibody or a fragment thereof. An anti-TDP-43 binding molecule is a molecule that binds to the TDP-43 protein, such as an anti-TDP-43 antibody or fragment thereof, at a specific recognition site, epitope. That is, antigen-binding molecules of the invention bind to an epitope within the amino acid sequence of SEQ ID NO: 1. The antigen-binding molecules, in particular antibodies or antigen-binding fragments thereof, provided herein recognize full-length TDP-43. Other anti-TDP-43 binding molecules may also include multivalent molecules, multi-specific molecules (e.g., diabodies), fusion molecules, aptamers, avimers, or other naturally occurring or recombinantly created molecules. Illustrative antigen-binding molecules useful in the present invention include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

The terms “anti TDP-43 antibody” and “an antibody that binds to TDP-43” or simply “antibody” as used herein refer to an antibody that is capable of binding TDP-43 with sufficient affinity such that the antibody is useful as a diagnostic and/or therapeutic agent in targeting TDP-43. In general, the term “antibody” is used herein in the broadest sense and encompasses various antibody structures, including but not limited to monoclonal antibodies, polyclonal antibodies, multi-specific antibodies (e.g., bispecific or biparatopic antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T cell.

An “antigen-binding fragment” of an antibody, or “functional fragment thereof” refers to a molecule other than an intact, or full-length, antibody that comprises a portion of an intact, or full-length, antibody and that binds (fully or partially) the antigen to which the intact, or full-length, antibody binds. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments. Antigen-binding fragments may also be referred to as “functional fragments” as they retain the binding function of the original antibody from which they are derived.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of TDP-43. Said binding/interaction is also understood to define a “specific recognition”. The term “specifically recognizing” means in accordance with this invention that the antibody is capable of specifically interacting with and/or binding to at least two amino acids of TDP-43 as defined herein, in particular interacting with/binding to at least two amino acids within amino acids residues 397-411 of human TDP-43 (SEQ ID NO: 1), even more particularly interacting with binding to at least two amino acids within amino acids residues 400-405, 400-406 or 400-412 of human TDP-43 (SEQ ID NO: 1).

The term “pan TDP-43 antibody” refers to an antibody that binds to misfolded aggregated TDP-43 and non-aggregated physiological TDP-43, including monomeric TDP-43, oligomeric TDP-43, post-translationally modified TDP-43 (such as phosphorylated, ubiquitinated, acetylated, sumoylated, and/or methylated), aggregated TDP-43 and truncated TDP-43.

The term “specific interaction” as used in accordance with the present invention means that the antibody or antigen-binding fragment thereof of the invention does not or does not essentially cross-react with (poly)peptides of similar structures. Accordingly, the antibody or antigen-binding fragment thereof of the invention specifically binds to/interacts with structures of TDP-43 formed by particular amino acid sequences within amino acids residues 397-411 of human TDP-43 (SEQ ID NO: 1), more particularly binds to/interacts with structures of TDP-43 formed by particular amino acid sequences within amino acids residues 400-405, 400-406 or 400-412 of human TDP-43 (SEQ ID NO: 1).

Cross-reactivity of antigen-binding molecules, in particular a panel of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of TDP-43 as defined herein, e.g., a specific epitope or (poly)peptide/protein of TDP-43 as defined herein but do not or do not essentially bind to any of the other epitope or (poly)peptides of the same TDP-43, are considered specific for the epitope or (poly)peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIACORE™), analytical ultracentrifugation, isothermal titration calorimetry, fluorescence anisotropy, fluorescence spectroscopy or by radiolabeled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant (human) antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The present invention specifically relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Thus, where reference is made herein to particular human framework sequences, such as an IGHV1-3 VH framework sequence, this is intended to encompass not simply the germline sequence but also mutated versions. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones et al., Nature 321 (1986), 522-525; Reichmann Nature 332 (1998), 323-327 and Presta Curr Op Struct Biol 2 (1992), 593-596. Reference may be made to Example 1 for a description of antibody humanization methods that may be employed according to the invention, including specific mutations.

A popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in the context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins.

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the VH and V_{L}domains which enables the scFv to form the desired structure for antigen binding. Techniques described for the production of single chain antibodies are described, e.g., in Pluckthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H}domain and the C_{H}1 domain and also the region between the C_{H}1 and C H₂ domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Humanized antibodies, humanized antibody constructs, humanized antibody fragments, humanized antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., Molecular Cloning: A Laboratory Manual; Cold Spring Harbor Laboratory Press, 2^{nd} edition (1989) and 3^{rd} edition (2001). The term “Ig-derived domain” particularly relates to (poly)peptide constructs comprising at least one CDR. Fragments or derivatives of the recited Ig-derived domains define (poly)peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991). CDR sequences provided herein are defined according to Kabat. However, it will be understood by the skilled person that the invention is intended to encompass binding molecules in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V, Chaume D, Bodmer J, Müller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc M P. Immunol Today. 1997 November; 18(11):509), MacCallum (MacCallum RM, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9. 10.1016/j.molimm.2008.05.022) numbering schemes may be adopted in order to define the CDRs.

Accordingly, in the context of the present invention, the humanized antibody molecule described herein above is selected from the group consisting of a full antibody (immunoglobulin, like an IgG1, an IgG2, an IgA1, an IgGA2, an IgG3, an IgG4, an IgA, an IgM, an IgD or an IgE), F(ab)-, Fab′-SH-, Fv-, Fab′-, F(ab′)₂- fragment, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

“Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art (see e.g. LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules or antigen-binding fragments thereof are provided, which are humanized and can successfully be employed in pharmaceutical compositions.

The specificity of the humanized antibody or antigen-binding fragment of the present invention may not only be expressed by the nature of the amino acid sequence of the humanized antibody or the antigen-binding fragment as defined above but also by the epitope to which the antibody is capable of binding. Thus, the present invention relates, in one embodiment, to an anti-misfolded humanized TDP-43 antibody or an antigen-binding fragment thereof which recognizes the same epitope as an antibody of the invention.

It may be understood by a person skilled in the art that the epitopes may be comprised in the TDP-43 protein, but may also be comprised in a degradation product thereof or may be a chemically synthesized peptide. The amino acid positions are only indicated to demonstrate the position of the corresponding amino acid sequence in the sequence of the TDP-43 protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 16 amino acids. The amino acids of such peptide may be natural amino acids or nonnatural amino acids (e.g., beta-amino acids, gamma-amino acids, D-amino acids) or a combination thereof.

Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane.

In order to test whether an antibody in question and the antibody of the present invention recognize the same epitope, the following competition study may be carried out: Vero cells infected with 3 MOI (multiplicity of infection) are incubated after 20 h with varying concentrations of the antibody in question as the competitor for 1 hour. In a second incubation step, the antibody of the present invention is applied in a constant concentration of 100 nM and its binding is flow-cytometrically detected using a fluorescence-labelled antibody directed against the constant domains of the antibody of the invention. Binding that conducts anti-proportional (inversely proportional) to the concentration of the antibody in question is indicative that both antibodies recognize the same epitope. However, many other assays are known in the art which may be used.

The present invention also relates to the production of specific antibodies against native polypeptides and recombinant polypeptides of TDP-43. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of TDP-43 can be subcloned into an appropriate vector, wherein the recombinant polypeptide is to be expressed in an organism capable of expression, for example in bacteria. Thus, the expressed recombinant protein can be intra-peritoneally injected into mice and the resulting specific antibody can be, for example, obtained from the mice serum being provided by intra-cardiac blood puncture. The present invention also envisages the production of specific antibodies against native polypeptides and recombinant polypeptides by using a DNA/RNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of TDP-43, in particular the epitope of the antibodies provided herein, can be obtained by directly immunizing the animal by directly injecting intramuscularly the vector expressing the desired polypeptide or a protein or an epitope of TDP-43, in particular the epitope of the antibodies of the invention, which lies within amino acid residues 397-411 of SEQ ID NO:1, more particularly which lies within amino acid residues 400-405, 400-406 or 400-412 of SEQ ID NO:1. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

Thus, under designated assay conditions, the specified antibodies and the corresponding epitope of TDP-43 bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell, for a description of immunoassay formats and conditions that can be used to determine specific immunoreactivity. Typically, a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 6, F, 7, and, respectively.

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs.

Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity). Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighbouring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties. In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e. g. complex, hybrid and high mannose structures) as measured by MALDI-TOF mass spectrometry, as described in WO2008/077546, for example. Asn297 refers to the asparagine residue located at about position 297 in the Fc region (Eu numbering of Fc region residues; see Edelman, G. M. et al., Proc. Natl. Acad. USA, 63, 78-85 (1969)); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L); US 2004/0093621 (Kyowa Hakko Kogyo Co., Ltd). Examples of publications related to “defucosylated” or “fucose deficient” antibody variants include: US2003/0157108; WO2000/61739; WO2001/29246; US2003/0115614; US2002/0164328; US2004/0093621; US2004/0132140; US2004/0110704; US 2004/0110282; US2004/0109865; WO2003/085119; WO2003/084570; WO2005/035586; WO2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO2004/056312 A1, Adams et al., especially at Example 11), and knockout cell lines, such as alpha-1,6-fucosyltransferase gene, FUT8, knockout CHO cells (see, e.g., Yamane-Ohnuki et al., Bioteeh. Bioeng. 87: 614 (2004); Kanda, Y. et al., Bioteehnol. Bioeng., 94(4):680-688 (2006); and WO2003/085 107). Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the antibody provided herein binds to pathological TDP-43 and forms an immune complex that may be cleared by antibody-dependent cellular phagocytosis (ADCP), which as a result potentiates TDP-43 clearance. ADCP is mediated by the interaction of antibody Fc fragment with Fc receptors, such as Fc gamma receptors, expressed at the surface of innate immune cells, such as microglia or dendritic cells. Fc mediated functions may be modulated to achieve the desired effect by modifying the Fc portion of antibodies.

In certain embodiments, the invention contemplates an antibody variant that possesses some but not all effector functions, which make it a desirable candidate for applications in which the half-life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcTR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al., Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Alternatively, non-radioactive assays methods may be employed (see, for example, ACTI™ non radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells.

Alternatively, or additionally, ADCC activity of the molecule of interest may be assessed in vivo, e.g., in an animal model such as that disclosed in Clynes et al., Proc. Nat'l Acad. sci. USA 95:652-656 (1998).

C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” Fc mutant with substitution of residues 265 to alanine and 297 to glycine. Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908). Other known mutations at position 234, 235 and 321, the so-called™ mutant containing mutations L234F/L235E/P331S in the CH2 domain, can be used (Oganesyan et al. Acta Cryst. D64, 700-704. (2008)). Antibodies from the human IgG4 isotype include mutations S228P/L235E to stabilize the hinge and to reduce FgR binding (Schlothauer et al, PEDS, 29 (10):457-466). Numbering of the constant domain is according to the EU numbering system.

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional nonproteinaceous moieties that are known in the art and readily available. The moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed. Antibodies may be produced using recombinant methods and compositions, e.g., as described in U.S. Pat. No. 4,816,567. In one embodiment, isolated nucleic acid encoding an anti-misfolded TDP-43 antibody described herein is provided. Such nucleic acid may encode an amino acid sequence comprising the VL and/or an amino acid sequence comprising the VH of the antibody (e.g., the Light and/or Heavy Chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an anti-misfolded TDP-43 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-misfolded TDP-43 antibody, nucleic acid encoding an antibody, e.g., as described above, is isolated and inserted into one or more vectors for further cloning and/or expression in a host cell or a cell-free expression system. Such nucleic acid may be readily isolated and sequenced using conventional procedures (e.g., by using oligonucleotide probes that are capable of binding specifically to genes encoding the Heavy and Light Chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Val. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CV1 line transformed by SV40 (COS-7); human embryonic kidney line (293 or 293 cells as described, e.g., in Graham et al., J. Gen Viral. 36:59 (1977)); baby hamster kidney cells (BHK); mouse Sertoli cells (TM4 cells as described, e.g., in Mather, Biol. Reprod. 23:243-251 (1980)); macaque kidney cells (CV 1); African green macaque kidney cells (VERO-76); human cervical carcinoma cells (HeLa); canine kidney cells (MDCK); buffalo rat liver cells (BRL 3A); human lung cells (WI38); human liver cells (Hep G2); mouse mammary tumor (MMT 060562); TRI cells, as described, e.g., in Mather et al., Annals N.Y Aead. Sei. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. cii. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Val. 248 (B. K. C. Lo, ed., Humana Press, Totowa, NJ), pp. 255-268 (2003).

Several art-known approaches exist for delivering molecules across the blood brain barrier (BBB) such as alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gill et. al.., Nature Med. 9: 589-595 (2003); and Gliadel Wafers™, Guildford Pharmaceutical), and intranasal administration to bypass the BBB (Mittal et al, Drug Deliv.21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

Trojan horse delivery methods of delivering the humanized antibody or humanized antibody fragment thereof across the blood-brain barrier include, but are not limited to, cationizing the antibodies (see, e.g., U.S. Pat. No. 5,004,697), and the use of cell-penetration peptides such as Tat peptides to gain entry into the CNS. (see, e.g. Dietz et al., J. Neurochem. 104:757-765 (2008)).

Nanoparticle delivery methods of delivering the humanized antibody or antigen-binding fragment thereof across the blood brain barrier include, but are not limited to, encapsulating the antibody or antigen-binding fragment thereof in liposomes, or extracellular vesicles such as exosomes, that are coupled to without limitation antibody or antigen-binding fragments or alternatively peptides that bind to receptors on the vascular endothelium of the blood-brain barrier (see, e.g., U.S. Patent Application Publication No. 20020025313), and coating the antibody or antigen-binding fragment thereof in low-density lipoprotein particles (see, e.g., U.S. Patent Application Publication No. 20040204354) or apolipoprotein E (see, e.g., U.S. Patent Application Publication No. 20040131692).

Humanized antibodies of the invention can be further modified to enhance blood brain barrier penetration.

The humanized antibody or antigen-binding fragment thereof of the invention can be fused to a polypeptide binding to a blood-brain barrier receptor. BBB receptors include, but are not limited to, transferrin receptor, insulin receptor or low-density lipoprotein receptor. The polypeptide can be a peptide, a receptor ligand, a single domain antibody (VHH), a scFv or a Fab fragment.

Humanized antibodies of the invention can also be delivered as a corresponding nucleic acid encoding for the humanized antibody. Such nucleic acid molecule may be a part of a viral vector for targeted delivery to the blood-brain barrier or any other cell type in the CNS. A viral vector may be a recombinant adeno-associated viral vector (rAAV) selected from any AAV serotype known in the art, including, without limitation, from AAV1 to AAV12 to enable the humanized antibody or humanized antibody fragment or humanized antibody derivatives to be expressed intracellularly or into the brain parenchyma.

Cell therapy methods of delivering the humanized antibody of the invention or humanized antibody fragment or humanized antibody derivatives across the blood brain barrier include, but are not limited to, the use of the homing capacity of Endothelial Progenitor Cells (EPCs) transfected ex vivo with our proprietary vectors and the secretion and delivery of antibodies or antibody fragments to the brain by these cells, to overcome the powerful filtering activity of the BBB (see, e.g., Heller and al., J Cell Mol Med. 00:1-7 (2020)), or the use of polymeric cell implant devices loaded with genetically engineered cells, to secrete antibody or antibody fragments (see, e.g. Marroquin Belaunzaran et al. PLoS ONE 6(4): e18268 (2011)).

Pharmaceutically acceptable carriers, diluents, adjuvants and excipients are well known in the pharmaceutical art and are described, for example, in Remington's Pharmaceutical Sciences, 15th or 18th Ed. (Alfonso R. Gennaro, ed.; Mack Publishing Company, Easton, PA, 1990); Remington: the Science and Practice of Pharmacy 19th Ed. (Lippincott, Williams & Wilkins, 1995); Handbook of Pharmaceutical Excipients, 3rd Ed. (Arthur H. Kibbe, ed.; Amer. Pharmaceutical Assoc, 1999); Pharmaceutical Codex: Principles and Practice of Pharmaceutics 12th Ed. (Walter Lund ed.; Pharmaceutical Press, London, 1994); The United States Pharmacopeia: The National Formulary (United States Pharmacopeial Convention); Fiedler's “Lexikon der Hilfstoffe” 5th Ed., Edition Cantor Verlag Aulendorf 2002; “The Handbook of Pharmaceutical Excipients”, 4th Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof. See Remington's Pharmaceutical Sciences, 15th or 18th Ed. (Alfonso R. Gennaro, ed.; Mack Publishing Company, Easton, PA, 1990); Remington: the Science and Practice of Pharmacy 19th Ed. (Lippincott, Williams & Wilkins, 1995); Handbook of Pharmaceutical Excipients, 3rd Ed. (Arthur H. Kibbe, ed.; Amer. Pharmaceutical Assoc, 1999); Pharmaceutical Codex: Principles and Practice of Pharmaceutics 12th Ed. (Walter Lund ed.; Pharmaceutical Press, London, 1994); The United States Pharmacopeia: The National Formulary (United States Pharmacopeial Convention); Fiedler's “Lexikon der Hilfstoffe” 5th Ed., Edition Cantor Verlag Aulendorf 2002; “The Handbook of Pharmaceutical Excipients”, 4th Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The “effective amount” of the compound which is to be administered to a subject is the dosage which according to sound medical judgement is suitable for treating, preventing or alleviating the disease, disorder or abnormality. The specific dose level and frequency of dosage can depend, e.g., upon a variety of factors including the activity of the specific compound employed, the metabolic stability and length of action of that compound, mode and time of administration. The “effective amount” of the compound which is to be administered to a subject is the dosage which according to sound medical judgement is suitable for treating, preventing or alleviating the disorder, disease, disorder or abnormality. The specific dose level and frequency of dosage can depend, e.g., upon a variety of factors including the activity of the specific compound employed, the metabolic stability and length of action of that compound, mode and time of administration, the rate of excretion, and drug combination. Patient-specific factors such as the age, body weight, general health, sex, diet, as well as the severity of the particular condition can also influence the amount which is to be administered.

The term “clearance” (also referred as “clearance value” or “CL” or “systemic clearance”) relates to the efficiency of elimination of a substance from the body. Clearance of a substance (in this case a binding molecule of the invention) is the sum of the urinary and extrarenal clearance; for substances that are eliminated by renal and extrarenal routes, plasma clearance exceeds urinary clearance. The PK properties of mAbs are a function of their large size (150 kDa), relative polarity, Fc-receptor binding and specific binding to target antigens. The primary elimination route for mAbs is cellular uptake followed by proteolytic degradation. Low clearance of mAbs from the systemic circulation enables them to be administered less frequently than peptides or small molecules, which is often more convenient for patients (Betts et al., MABs. 2018).

### XI. Invention Embodiments for Tdp-43 Specific Binding Molecule

In some embodiments, a humanized TDP-43 binding molecule, in particular a humanized TDP-43 antibody or antigen-binding fragment thereof is provided which comprises:
a) VH-CDR1 comprising the amino acid sequence of SEQ ID NO: 21; VH-CDR2 comprising the amino acid sequence of SEQ ID NO: 22; and VH-CDR3 comprising the amino acid sequence ES (Glu-Ser); or
b) VH-CDR1 comprising the amino acid sequence of SEQ ID NO: 21; VH-CDR2 comprising the amino acid sequence of SEQ ID NO: 82; and VH-CDR3 comprising the amino acid sequence ES (Glu-Ser); or
c) VH-CDR1 comprising the amino acid sequence of SEQ ID NO: 21; VH-CDR2 comprising the amino acid sequence of SEQ ID NO: 92; and VH-CDR3 comprising the amino acid sequence ES (Glu-Ser).
d) VH-CDR1 comprising the amino acid sequence of SEQ ID NO: 21; VH-CDR2 comprising the amino acid sequence of SEQ ID NO: 102; and VH-CDR3 comprising the amino acid sequence ES (Glu-Ser); or
e) VH-CDR1 comprising the amino acid sequence of SEQ ID NO: 21; VH-CDR2 comprising the amino acid sequence of SEQ ID NO: 112; and VH-CDR3 comprising the amino acid sequence ES (Glu-Ser); or
f) VH-CDR1 comprising the amino acid sequence of SEQ ID NO: 21; VH-CDR2 comprising the amino acid sequence of SEQ ID NO: 122; and VH-CDR3 comprising the amino acid sequence ES (Glu-Ser).

In some embodiments, a humanized TDP-43 binding molecule, in particular a humanized TDP-43 antibody or antigen-binding fragment thereof is provided which comprises:
a) VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 27; or
b) VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87; or
c) VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 87.

In some embodiments, a humanized TDP-43 binding molecule, in particular a humanized TDP-43 antibody or antigen-binding fragment thereof is provided which comprises:
a) a Heavy Chain Variable Region (VH) comprising:
   i. a VH-CDR1 comprising the amino acid sequence of SEQ ID NO: 21; a VH-CDR2 comprising the amino acid sequence of SEQ ID NO: 22; and a VH-CDR3 comprising the amino acid sequence ES (Glu-Ser); or
   ii. a VH-CDR1 comprising the amino acid sequence of SEQ ID NO: 21; a VH-CDR2 comprising the amino acid sequence of SEQ ID NO: 82; and a VH-CDR3 comprising the amino acid sequence ES (Glu-Ser); or
   iii. a VH-CDR1 comprising the amino acid sequence of SEQ ID NO: 21; a VH-CDR2 comprising the amino acid sequence of SEQ ID NO: 92; and a VH-CDR3 comprising the amino acid sequence ES (Glu-Ser); or
   iv. a VH-CDR1 comprising the amino acid sequence of SEQ ID NO: 21; a VH-CDR2 comprising the amino acid sequence of SEQ ID NO: 102; and a VH-CDR3 comprising the amino acid sequence ES (Glu-Ser); or
   v. a VH-CDR1 comprising the amino acid sequence of SEQ ID NO: 21; a VH-CDR2 comprising the amino acid sequence of SEQ ID NO: 112; and a VH-CDR3 comprising the amino acid sequence ES (Glu-Ser); or
   vi. a VH-CDR1 comprising the amino acid sequence of SEQ ID NO: 21; a VH-CDR2 comprising the amino acid sequence of SEQ ID NO: 122; and a VH-CDR3 comprising the amino acid sequence ES (Glu-Ser); and
b) a Light Chain Variable Region (VL) comprising:
   i. VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 27; or
   ii. VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87; or
   iii. VL-CDR1 comprising the amino acid sequence of SEQ ID NO: 25; VL-CDR2 comprising the amino acid sequence of SEQ ID NO: 16; and VL-CDR3 comprising the amino acid sequence of SEQ ID NO: 87.

In some embodiments, a humanized TDP-43 binding molecule, in particular a humanized TDP-43 antibody or antigen-binding fragment thereof is provided which comprises:
a) a Heavy Chain Variable Region (VH) comprising:
   i. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence ES (Glu-Ser); or
   ii. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 82 or a VH-CDR2 comprising an amino acid sequence having at least 80%, 90%, 95% or 100% sequence identity to SEQ ID NO: 82; and a VH-CDR3 comprising the amino acid sequence ES (Glu-Ser); or
   iii. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 92 or a VH-CDR2 comprising an amino acid sequence having at least 80%, 90%, 95% or 100% sequence identity to SEQ ID NO: 92; and a VH-CDR3 comprising the amino acid sequence ES (Glu-Ser); or
   iv. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 102 or a VH-CDR2 comprising an amino acid sequence having at least 80%, 90%, 95% or 100% sequence identity to SEQ ID NO: 102; and a VH-CDR3 comprising the amino acid sequence ES (Glu-Ser); or
   v. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 112 or a VH-CDR1 comprising an amino acid sequence having at least 80%, 90%, 95% or 100% sequence identity to SEQ ID NO: 112; and a VH-CDR3 comprising the amino acid sequence ES (Glu-Ser); or
   vi. a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; a VH-CDR2 comprising the amino acid sequence of SEQ ID NO: 122 or a VH-CDR2 comprising an amino acid sequence having at least 80%, 90%, 95% or 100% sequence identity to SEQ ID NO: 122; and a VH-CDR3 comprising the amino acid sequence ES (Glu-Ser); and
b) a Light Chain Variable Region (VL) comprising:
   i. VL-CDR1 comprising the amino acid sequence of SEQ ID NO: 25 or a VL-CDR1 comprising an amino acid sequence having at least 80%, 90%, 95% or 100% sequence identity to SEQ ID NO: 25; VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
   ii. VL-CDR1 comprising the amino acid sequence of SEQ ID NO: 85 or a VL-CDR1 comprising an amino acid sequence having at least 80%, 90%, 95% or 100% sequence identity to SEQ ID NO: 85; VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and VL-CDR3 comprising the amino acid sequence of SEQ ID NO: 87 or a VL-CDR1 comprising an amino acid sequence having at least 80%, 90%, 95% or 100% sequence identity to SEQ ID NO: 87; or
   iii. VL-CDR1 comprising the amino acid sequence of SEQ ID NO: 25 or a VL-CDR1 comprising an amino acid sequence having at least 80%, 90%, 95% or 100% sequence identity to SEQ ID NO: 25; VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

In some embodiments, a humanized TDP-43 binding molecule, in particular a humanized TDP-43 antibody or antigen-binding fragment thereof is provided which comprises:
a) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; aVH-CDR2 comprising the amino acid sequence of SEQ ID NO: 22 or a VH-CDR2 comprising an amino acid sequence having at least 80%, 90%, 95% or 100% sequence identity to SEQ ID NO: 22; and a VH-CDR3 comprising the amino acid sequence ES (Glu-Ser); or
b) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; aVH-CDR2 comprising the amino acid sequence of SEQ ID NO: 112 or a VH-CDR2 comprising an amino acid sequence having at least 80%, 90%, 95% or 100% sequence identity to SEQ ID NO: 112; and a VH-CDR3 comprising the amino acid sequence ES (Glu-Ser);
c) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO: 21; aVH-CDR2 comprising the amino acid sequence of SEQ ID NO: 122 or a VH-CDR2 comprising an amino acid sequence having at least 80%, 90%, 95% or 100% sequence identity to SEQ ID NO: 122; and a VH-CDR3 comprising the amino acid sequence ES (Glu-Ser); and
d) a Light Chain Variable Region (VL) comprising: a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 27 or a VL-CDR3 comprising an amino acid sequence having at least 80%, 90%, 95% or 100% sequence identity to SEQ ID NO: 27; or
e) a Light Chain Variable Region (VL) comprising: a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87; or
f) a Light Chain Variable Region (VL) comprising: a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

More specifically, in some embodiments, a humanized TDP-43 binding molecule, in particular a humanized TDP-43 antibody or antigen-binding fragment thereof is provided which comprises:
a) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO 21; aVH-CDR2 comprising the amino acid sequence of SEQ ID NO: 122 or a VH-CDR2 comprising an amino acid sequence having at least 80%, 90%, 95% or 100% sequence identity to SEQ ID NO: 122; and a VH-CDR3 comprising the amino acid sequence ES (Glu-Ser); and
b) a Light Chain Variable Region (VL) comprising: a VL-CDR1 comprising the amino acid sequence of SEQ ID NO: 85 or a VL-CDR1 comprising an amino acid sequence having at least 80%, 90%, 95% or 100% sequence identity to SEQ ID NO: 85; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

More specifically, in some embodiments, a humanized TDP-43 binding molecule, in particular a humanized TDP-43 antibody or antigen-binding fragment thereof is provided which comprises:
a) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO 21; aVH-CDR2 comprising the amino acid sequence of SEQ ID NO: 112 or a VH-CDR2 comprising an amino acid sequence having at least 80%, 90%, 95% or 100% sequence identity to SEQ ID NO: 112; and a VH-CDR3 comprising the amino acid sequence ES (Glu-Ser); and
b) a Light Chain Variable Region (VL) comprising: a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87.

More specifically, in some embodiments, a humanized TDP-43 binding molecule, in particular a humanized TDP-43 antibody or antigen-binding fragment thereof is provided which comprises:
a) a Heavy Chain Variable Region (VH) comprising a VH-CDR1 comprising the amino acid sequence of SEQ ID NO: 21 or a VH-CDR1 comprising an amino acid sequence having at least 80%, 90%, 95% or 100% sequence identity to SEQ ID NO 21; aVH-CDR2 comprising the amino acid sequence of SEQ ID NO: 122 or a VH-CDR2 comprising an amino acid sequence having at least 80%, 90%, 95% or 100% sequence identity to SEQ ID NO: 122; and a VH-CDR3 comprising the amino acid sequence ES (Glu-Ser); and
a Light Chain Variable Region (VL) comprising: a VL-CDR1 comprising the amino acid sequence of SEQ ID NO: 25 or a VL-CDR1 comprising an amino acid sequence having at least 80%, 90%, 95% or 100% sequence identity to SEQ ID NO: 25; a VL-CDR2 comprising the amino acid sequence of SEQ ID NO: 16 or a VL-CDR2 comprising an amino acid sequence having at least 80%, 90%, 95% or 100% sequence identity to SEQ ID NO: 16; and a VL-CDR3 comprising the amino acid sequence of SEQ ID NO: 87 or a VL-CDR3 comprising an amino acid sequence having at least 80%, 90%, 95% or 100% sequence identity to SEQ ID NO: 87. In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 22, SEQ ID NO: 112 or SEQ ID NO: 122; (c) VH-CDR3 comprising the amino acid sequence ES (Glu-Ser); (d) VL-CDR1 comprising the amino acid sequence of SEQ ID NO: 25 or SEQ ID NO: 85; (e) VL-CDR2 comprising the amino acid sequence of SEQ ID NO: 16; and (f) VL-CDR3 comprising the amino acid sequence of SEQ ID NO: 27 or SEQ ID NO: 87.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 22; (c) VH-CDR3 comprising the amino acid sequence ES (Glu-Ser); (d) VL-CDR1 comprising the amino acid sequence of SEQ ID NO: 25; (e) VL-CDR2 comprising the amino acid sequence of SEQ ID NO: 16; and (f) VL-CDR3 comprising the amino acid sequence of SEQ ID NO: 27.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 112; (c) VH-CDR3 comprising the amino acid sequence ES (Glu-Ser); (d) VL-CDR1 comprising the amino acid sequence of SEQ ID NO: 25; (e) VL-CDR2 comprising the amino acid sequence of SEQ ID NO: 16; and (f) VL-CDR3 comprising the amino acid sequence of SEQ ID NO: 27.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 112; (c) VH-CDR3 comprising the amino acid sequence ES (Glu-Ser); (d) VL-CDR1 comprising the amino acid sequence of SEQ ID NO: 85; (e) VL-CDR2 comprising the amino acid sequence of SEQ ID NO: 16; and (f) VL-CDR3 comprising the amino acid sequence of SEQ ID NO: 87.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 122; (c) VH-CDR3 comprising the amino acid sequence ES (Glu-Ser); (d) VL-CDR1 comprising the amino acid sequence of SEQ ID NO: 25; (e) VL-CDR2 comprising the amino acid sequence of SEQ ID NO: 16; and (f) VL-CDR3 comprising the amino acid sequence of SEQ ID NO: 27.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 122; (c) VH-CDR3 comprising the amino acid sequence ES (Glu-Ser); (d) VL-CDR1 comprising the amino acid sequence of SEQ ID NO: 85; (e) VL-CDR2 comprising the amino acid sequence of SEQ ID NO: 16; and (f) VL-CDR3 comprising the amino acid sequence of SEQ ID NO: 87.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 112; (c) VH-CDR3 comprising the amino acid sequence ES (Glu-Ser); (d) VL-CDR1 comprising the amino acid sequence of SEQ ID NO: 25; (e) VL-CDR2 comprising the amino acid sequence of SEQ ID NO: 16; and (f) VL-CDR3 comprising the amino acid sequence of SEQ ID NO: 87.

In some embodiments, a humanized TDP-43 antibody comprises CDRs selected from (a) VH-CDR1 comprising the amino acid sequence of SEQ ID NO: 21; (b) VH-CDR2 comprising the amino acid sequence of SEQ ID NO: 122; (c) VH-CDR3 comprising the amino acid sequence ES (Glu-Ser); (d) VL-CDR1 comprising the amino acid sequence of SEQ ID NO: 25; (e) VL-CDR2 comprising the amino acid sequence of SEQ ID NO: 16; and (f) VL-CDR3 comprising the amino acid sequence of SEQ ID NO: 87.

In another embodiment, a humanized TDP-43 antibody comprises a Heavy Chain Variable Domain (VH) selected from the group consisting of SEQ ID NO: 30, 40, 50, 60, 70, 80, 90, 100, 110 and 120, and including post-translational modifications of that sequence. In a particular embodiment, the Heavy Chain Variable Domain (VH) comprises at least one, two, or three CDRs selected from (a) VH-CDR1 comprising the amino acid sequence SEQ ID NO: 21, (b) VH-CDR2 comprising the amino acid sequence selected from the group consisting of SEQ ID NO: 22, 82, 92, 102, 112 and 122 (c) VH-CDR3 comprising the amino acid ES (Glu-Ser).

In another embodiment, a humanized TDP-43 antibody comprises a Light Chain Variable Domain (VL) selected from the group consisting of SEQ ID NO: 34, 44, 54, 64, 74, 84 and 94 and including post-translational modifications of that sequence. In a particular embodiment, the Light Chain Variable Domain (VL) comprises at least one, two, or three CDRs selected from (a) VL-CDR1 comprising the amino acid sequence selected from the group consisting of SEQ ID NO: 25, and SEQ ID NO: 85, and (b) VL-CDR2 comprising the amino acid sequence SEQ ID NO: 16 and (c) VL-CDR3 comprising the amino acid sequence selected from the group consisting of SEQ ID NO: 27 and 87.

In some embodiments, the humanized TDP-43 antibody comprises:
a. a Heavy Chain Variable Region (VH) comprising the sequence of SEQ ID NO: 30 or a Heavy Chain Variable Region (VH) having at least 91%, 92%, 93%, 94%, 95%, 96%, 97%, 98% or 99% sequence identity to the amino acid sequence of SEQ ID NO: 30; or
b. a Heavy Chain Variable Region (VH) comprising the sequence of SEQ ID NO: 40 or a Heavy Chain Variable Region (VH) having at least 91%, 92%, 93%, 94%, 95%, 96%, 97%, 98% or 99% sequence identity to the amino acid sequence of SEQ ID NO: 40; or
c. a Heavy Chain Variable Region (VH) comprising the sequence of SEQ ID NO: 50 or a Heavy Chain Variable Region (VH) having at least 91%, 92%, 93%, 94%, 95%, 96%, 97%, 98%, or 99% sequence identity to the amino acid sequence of SEQ ID NO: 50; or
d. a Heavy Chain Variable Region (VH) comprising the sequence of SEQ ID NO: 60 or a Heavy Chain Variable Region (VH) having at least 91%, 92%, 93%, 94%, 95%, 96%, 97%, 98% or 99% sequence identity to the amino acid sequence of SEQ ID NO: 60; or
e. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; or
f. a Heavy Chain Variable Region (VH) comprising the sequence of SEQ ID NO: 80 or a Heavy Chain Variable Region (VH) having at least 91%, 92%, 93%, 94%, 95%, 96%, 97%, 98% or 99% sequence identity to the amino acid sequence of SEQ ID NO: 80; or
g. a Heavy Chain Variable Region (VH) comprising the sequence of SEQ ID NO: 90 or a Heavy Chain Variable Region (VH) having at least 91%, 92%, 93%, 94%, 95%, 96%, 97%, 98% or 99% sequence identity to the amino acid sequence of SEQ ID NO: 90; or
h. a Heavy Chain Variable Region (VH) comprising the sequence of SEQ ID NO: 100 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 100; or
i. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; or
j. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120.

In some embodiments, the humanized TDP-43 antibody comprises:
a. a Light Chain Variable Region (VL) comprising the sequence of SEQ ID NO: 34 or a Light Chain Variable Region (VL) having at least 97%, 98% or 99% sequence identity to the amino acid sequence of SEQ ID NO: 34; or
b. a Light Chain Variable Region (VL) comprising the sequence of SEQ ID NO: 44 or a Light Chain Variable Region (VL) having at least 96%, 97%, 98% or 99% sequence identity to the amino acid sequence of SEQ ID NO: 44; or
c. a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
d. a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
e. a Light Chain Variable Region (VL) comprising the sequence of SEQ ID NO: 74 or a Light Chain Variable Region (VL) having at least 96%, 97%, 98% or 99% sequence identity to the amino acid sequence of SEQ ID NO: 74; or
f. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
g. a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94.

In some embodiment, the humanized TDP-43 binding molecule, in particular a humanized TDP-43 antibody or antigen-binding fragment comprises:
a) a Heavy Chain Variable Region (VH) selected from:
   i. a Heavy Chain Variable Region (VH) comprising the sequence of SEQ ID NO: 30 or a Heavy Chain Variable Region (VH) having at least 91%, 92%, 93%, 94%, 95%, 96%, 97%, 98% or 99% sequence identity to the amino acid sequence of SEQ ID NO: 30; or
   ii. a Heavy Chain Variable Region (VH) comprising the sequence of SEQ ID NO: 40 or a Heavy Chain Variable Region (VH) having at least 91%, 92%, 93%, 94%, 95%, 96%, 97%, 98% or 99% sequence identity to the amino acid sequence of SEQ ID NO: 40; or
   iii. a Heavy Chain Variable Region (VH) comprising the sequence of SEQ ID NO: 50 or a Heavy Chain Variable Region (VH) having at least 91%, 92%, 93%, 94%, 95%, 96%, 97%, 98%, or 99% sequence identity to the amino acid sequence of SEQ ID NO: 50; or
   iv. a Heavy Chain Variable Region (VH) comprising the sequence of SEQ ID NO: 60 or a Heavy Chain Variable Region (VH) having at least 91%, 92%, 93%, 94%, 95%, 96%, 97%, 98% or 99% sequence identity to the amino acid sequence of SEQ ID NO: 60; or
   v. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; or
   vi. a Heavy Chain Variable Region (VH) comprising the sequence of SEQ ID NO: 80 or a Heavy Chain Variable Region (VH) having at least 91%, 92%, 93%, 94%, 95%, 96%, 97%, 98% or 99% sequence identity to the amino acid sequence of SEQ ID NO: 80; or
   vii. a Heavy Chain Variable Region (VH) comprising the sequence of SEQ ID NO: 90 or a Heavy Chain Variable Region (VH) having at least 91%, 92%, 93%, 94%, 95%, 96%, 97%, 98% or 99% sequence identity to the amino acid sequence of SEQ ID NO: 90; or
   viii. a Heavy Chain Variable Region (VH) comprising the sequence of SEQ ID NO: 100 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 100; or
   ix. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; or
   x. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and
b) a Light Chain Variable Region (VL) selected from:
   i. a Light Chain Variable Region (VL) comprising the sequence of SEQ ID NO: 34 or a Light Chain Variable Region (VL) having at least 97%, 98% or 99% sequence identity to the amino acid sequence of SEQ ID NO: 34; or
   ii. a Light Chain Variable Region (VL) comprising the sequence of SEQ ID NO: 44 or a Light Chain Variable Region (VL) having at least 96%, 97%, 98% or 99% sequence identity to the amino acid sequence of SEQ ID NO: 44; or
   iii. a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
   iv. a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 54; or
   v. a Light Chain Variable Region (VL) comprising the sequence of SEQ ID NO: 74 or a Light Chain Variable Region (VL) having at least 96%, 97%, 98% or 99% sequence identity to the amino acid sequence of SEQ ID NO: 74; or
   vi. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
   vii. a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94.

In some embodiments, the humanized TDP-43 binding molecule, in particular a humanized TDP-43 antibody or antigen-binding fragment thereof comprises:
a. a Heavy Chain Variable Region (VH) comprising the sequence of SEQ ID NO: 60 or a Heavy Chain Variable Region (VH) having at least 91%, 92%, 93%, 94%, 95%, 96%, 97%, 98% or 99% sequence identity to the amino acid sequence of SEQ ID NO: 60; and a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
b. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the sequence of SEQ ID NO: 44 or a Light Chain Variable Region (VL) having at least 96%, 97%, 98% or 99% sequence identity to the amino acid sequence of SEQ ID NO: 44; or
c. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98%, or 99% sequence identity to the amino acid sequence of SEQ ID NO: 70; and a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
d. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
e. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
f. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
g. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
h. a Heavy Chain Variable Region (VH) comprising the sequence of SEQ ID NO: 120 or a Heavy Chain Variable Region (VH) having at least 91%, 92%, 93%, 94%, 95%, 96%, 97%, 98%, or 99% sequence identity to the amino acid sequence of SEQ ID NO: 120; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
i. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94; or
j. a Heavy Chain Variable Region (VH) comprising the sequence of SEQ ID NO: 120 or a Heavy Chain Variable Region (VH) having at least 91%, 92, 93%, 94%, 95%, 96%, 97%, 98% or 99% sequence identity to the amino acid sequence of SEQ ID NO: 120; and a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94.

In some embodiments, the humanized TDP-43 antibody comprises:
a. a Heavy Chain Variable Region (VH) comprising the sequence of SEQ ID NO: 60 and a Light Chain Variable Region (VL) comprising the sequence of SEQ ID NO: 54; or
b. a Heavy Chain Variable Region (VH) comprising the sequence of SEQ ID NO: 70 and a Light Chain Variable Region (VL) comprising the sequence of SEQ ID NO: 44; or
c. a Heavy Chain Variable Region (VH) comprising the sequence of SEQ ID NO: 70 and a Light Chain Variable Region (VL) comprising the sequence of SEQ ID NO: 54; or
d. a Heavy Chain Variable Region (VH) comprising the sequence of SEQ ID NO: 70 and a Light Chain Variable Region (VL) comprising the sequence of SEQ ID NO: 64; or
e. a Heavy Chain Variable Region (VH) comprising the sequence of SEQ ID NO: 110 and a Light Chain Variable Region (VL) comprising the sequence of SEQ ID NO: 64; or
f. a Heavy Chain Variable Region (VH) comprising the sequence of SEQ ID NO: 110 and a Light Chain Variable Region (VL) comprising the sequence of SEQ ID NO: 84; or
g. a Heavy Chain Variable Region (VH) comprising the sequence of SEQ ID NO: 120 and a Light Chain Variable Region (VL) comprising the sequence of SEQ ID NO: 64; or
h. a Heavy Chain Variable Region (VH) comprising the sequence of SEQ ID NO: 120 and a Light Chain Variable Region (VL) comprising the sequence of SEQ ID NO: 84; or
i. a Heavy Chain Variable Region (VH) comprising the sequence of SEQ ID NO: 110 and a Light Chain Variable Region (VL) comprising the sequence of SEQ ID NO: 94; or
j. a Heavy Chain Variable Region (VH) comprising the sequence of SEQ ID NO: 120 and a Light Chain Variable Region (VL) comprising the sequence of SEQ ID NO: 94.

In some embodiments, the humanized TDP-43 antibody comprises:
a. a Heavy Chain Variable Region (VH) comprising the sequence of SEQ ID NO: 120 and a Light Chain Variable Region (VL) comprising the sequence of SEQ ID NO: 84; or
b. a Heavy Chain Variable Region (VH) comprising the sequence of SEQ ID NO: 110 and a Light Chain Variable Region (VL) comprising the sequence of SEQ ID NO: 94; or
c. a Heavy Chain Variable Region (VH) comprising the sequence of SEQ ID NO: 120 and a Light Chain Variable Region (VL) comprising the sequence of SEQ ID NO: 94

In some embodiments, the humanized TDP-43 antibody comprises:
a. a Heavy Chain Variable Region (VH) comprising the sequence of SEQ ID NO: 110 and a Light Chain Variable Region (VL) comprising the sequence of SEQ ID NO: 84; or
b. a Heavy Chain Variable Region (VH) comprising the sequence of SEQ ID NO: 110 and a Light Chain Variable Region (VL) comprising the sequence of SEQ ID NO: 94; or
c. a Heavy Chain Variable Region (VH) comprising the sequence of SEQ ID NO: 120 and a Light Chain Variable Region (VL) comprising the sequence of SEQ ID NO: 94.

In some embodiments, the invention relates to the humanized TDP-43 binding molecule selected from hACI-7069-633B12-Ab1_H27L23, hACI-7069-633B12-Ab1_H28L22, hACI-7069-633B12-Ab1_H28L23, hACI-7069-633B12-Ab1_H28L24, hACI-7069-633B12-Ab1_H32L24, hACI-7069-633B12-Ab1_H32L26, hACI-7069-633B12-Ab1_H33L24, hACI-7069-633B12-Ab1_H33L26, hACI-7069-633B12-Ab1_H32L27 or hACI-7069-633B12-Ab1_H33L27.

Preferably, the humanized TDP-43 binding molecule is selected from the group comprising hACI-7069-633B12-Ab1_H33L26, hACI-7069-633B12-Ab1_H32L27 or hACI-7069-633B12-Ab1_H33L27.

Even more preferably, the humanized TDP-43 binding molecule is selected from the group comprising hACI-7069-633B12-Ab1_H32L26, hACI-7069-633B12-Ab1_H32L27 or hACI-7069-633B12-Ab1_H33L27.

In one preferred embodiment, the humanized TDP-43 binding molecule is hACI-7069-633B12-Ab1_H33L27.

In some embodiments, the humanized TDP-43 binding molecule of the invention, in particular a humanized TDP-43 antibody or antigen-binding fragment thereof, comprises an Fv having a pI of below 7.8, preferably below 7.5, more preferably below 7.0.

In some embodiments, the humanized TDP-43 binding molecule of the invention, in particular a humanized TDP-43 antibody or antigen-binding fragment thereof, comprises an Fv having a net charge at pH 7.4 below 0.2, preferably below −0.8, more preferably below −1.8.

In some embodiments, the humanized TDP-43 binding molecule of the invention, in particular a humanized TDP-43 antibody or antigen-binding fragment thereof comprises an Fv having a pI below 7.0 and a net charge below −1.8.

In some embodiments of the invention, the humanized anti-TDP-43 binding molecules of the invention have a clearance in mice of equal to or less than 0.50 mL/h/kg, particularly equal to or less than 0.25 mL/h/kg, more particularly equal to or less than 0.18 mL/h/kg. In some embodiments, the humanized TDP-43 binding molecule of the invention, in particular a humanized TDP-43 antibody or antigen-binding fragment thereof, presents a half-life of at least 8 days in Tg32 mice, preferably at least 10 days, more preferably at least 12 days. In an embodiment, the humanized TDP-43 binding molecule may be hACI-7069-633B12-Ab1_H33L27 or hACI-7069-633B12-Ab1_H32L27. Reference can be made to Example 5 for the measurement of clearance (such as systemic, CL) and half-life (Ti/2 P).

In some embodiments of the invention, the humanized anti-TDP-43 binding molecules of the invention have a clearance in NHP of equal to or less than 0.27 mL/h/kg, particularly equal to or less than 0.12 mL/h/kg, more particularly equal to or less than 0.08 mL/h/kg. In some embodiments, the humanized TDP-43 binding molecule of the invention, in particular a humanized TDP-43 antibody or antigen-binding fragment thereof, presents a half-life of at least 6 days in NHP, preferably at least 8 days, for example at least 10 or 12 days. In an embodiment, the humanized TDP-43 binding molecule may be hACI-7069-633B12-Ab1_H33L27 or hACI-7069-633B12-Ab1_H32L27. Reference can be made to Example 6 for measurement of clearance (such as systemic, CL) and half-life (T_{1/2β}).

In some embodiments, the humanized TDP-43 binding molecule of the invention, in particular a humanized TDP-43 antibody or antigen-binding fragment thereof, presents a predicted human half-life of at least 20 days, preferably at least 24 days, for example at least 26 days. The predicted human half-life may be 27-30 days or 17-22 days. The humanized TDP-43 binding molecule may be hACI-7069-633B12-Ab1_H33L27 or hACI-7069-633B12-Ab1_H32L27. Reference can be made to Example 9 for how to calculate predicted human half-life from half-lives in Tg32 mice and/or cynomolgus monkeys.

In some embodiments, a(n isolated) nucleic acid is provided, wherein the (isolated) nucleic acid encodes a humanized TDP-43 binding molecule in particular humanized TDP-43 antibody and fragment thereof described herein.

In some embodiments, a(n isolated) nucleic acid is provided, wherein the (isolated) nucleic acid comprises SEQ ID NO:38 encoding a humanized anti-TPD-43 antibody.

In some embodiments, a(n isolated) nucleic acid is provided, wherein the (isolated) nucleic acid comprises SEQ ID NO:48 encoding a humanized anti-TPD-43 antibody.

In some embodiments, a(n isolated) nucleic acid is provided, wherein the (isolated) nucleic acid comprises SEQ ID NO:58 encoding a humanized anti-TPD-43 antibody.

In some embodiments, a(n isolated) nucleic acid is provided, wherein the (isolated) nucleic acid comprises SEQ ID NO:68 encoding a humanized anti-TPD-43 antibody.

In some embodiments, a(n isolated) nucleic acid is provided, wherein the (isolated) nucleic acid comprises SEQ ID NO:78 encoding a humanized anti-TPD-43 antibody.

In some embodiments, a(n isolated) nucleic acid is provided, wherein the (isolated) nucleic acid comprises SEQ ID NO:88 encoding a humanized anti-TPD-43 antibody.

In some embodiments, a(n isolated) nucleic acid is provided, wherein the (isolated) nucleic acid comprises SEQ ID NO:98 encoding a humanized anti-TPD-43 antibody.

In some embodiments, a(n isolated) nucleic acid is provided, wherein the (isolated) nucleic acid comprises SEQ ID NO:108 encoding a humanized anti-TPD-43 antibody.

In some embodiments, a(n isolated) nucleic acid is provided, wherein the (isolated) nucleic acid comprises SEQ ID NO:118 encoding a humanized anti-TPD-43 antibody.

In some embodiments, a(n isolated) nucleic acid is provided, wherein the (isolated) nucleic acid comprises SEQ ID NO:128 encoding a humanized anti-TPD-43 antibody.

In some embodiments, a(n isolated) nucleic acid is provided, wherein the (isolated) nucleic acid comprises SEQ ID NO:39 encoding a humanized anti-TPD-43 antibody.

In some embodiments, a(n isolated) nucleic acid is provided, wherein the (isolated) nucleic acid comprises SEQ ID NO:49 encoding a humanized anti-TPD-43 antibody.

In some embodiments, a(n isolated) nucleic acid is provided, wherein the (isolated) nucleic acid comprises SEQ ID NO:59 encoding a humanized anti-TPD-43 antibody.

In some embodiments, a(n isolated) nucleic acid is provided, wherein the (isolated) nucleic acid comprises SEQ ID NO:69 encoding a humanized anti-TPD-43 antibody.

In some embodiments, a(n isolated) nucleic acid is provided, wherein the (isolated) nucleic acid comprises SEQ ID NO:79 encoding a humanized anti-TPD-43 antibody.

In some embodiments, a(n isolated) nucleic acid is provided, wherein the (isolated) nucleic acid comprises SEQ ID NO:89 encoding a humanized anti-TPD-43 antibody.

In some embodiments, a(n isolated) nucleic acid is provided, wherein the (isolated) nucleic acid comprises SEQ ID NO:99 encoding a humanized anti-TPD-43 antibody.

### XII. Compositions and Methods

The invention also relates to pharmaceutical compositions comprising a humanized TDP-43 binding molecule, particularly a humanized antibody or an antigen-binding fragment thereof, of the invention as described herein and a pharmaceutically acceptable carrier and/or excipient and/or diluent.

In some embodiments, a pharmaceutical composition is provided, comprising an (isolated) humanized antibody described herein and a pharmaceutically acceptable carrier.

In some embodiments, a conjugated binding molecule, in particular antibody or antigen-binding fragment thereof, is provided, comprising a binding molecule, in particular an antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), nucleic acid molecules, detectable labels, therapeutic agents, toxins and blood brain barrier penetration moieties. Conjugation methods are well known in the art and several technologies are commercially available for conjugating antibodies to a label or other molecule, Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method. Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Conjugation may be via a suitable linker moiety to ensure suitable spatial separation of the antibody and conjugated molecule, such as detectable label. However, a linker may not be required in all instances. In some embodiments the humanized TDP-43 specific binding molecule of the present invention is linked to a detectable label.

The invention also relates to an immunoconjugate comprising the humanized TDP-43 binding molecule, provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood-brain barrier penetration moieties or detectable labels. Various techniques exist for improving drug delivery across the blood-brain barrier (BBB) as discussed herein, which discussion applies mutatis mutandis. Non-invasive techniques include the so-called “Trojan horse approach” in which conjugated molecules deliver the binding molecules of the invention by binding to BBB receptors and mediating transport. Suitable molecules may comprise endogenous ligands or antibodies, in particular monoclonal antibodies, that bind specific epitopes on the BBB receptor.

In some embodiments, an immunoconjugate is provided, wherein the immunoconjugate comprises an (isolated) humanized antibody described herein and a therapeutic agent. In some embodiments, a labelled humanized antibody is provided, comprising a humanized antibody described herein and a detectable label.

In some embodiments the humanized TDP-43 specific binding molecule is part of an immunoconjugate wherein the humanized TDP-43 specific binding molecule is covalently linked to another suitable therapeutic agent.

In some embodiments the humanized TDP-43 specific binding molecule or the immunoconjugate comprising it is present as a composition comprising a humanized TDP-43 specific binding molecule.

In some embodiments the humanized TDP-43 specific binding molecule is part of pharmaceutical composition comprising a humanized TDP-43 specific binding molecule, or an immunoconjugate wherein the humanized TDP-43 specific binding molecule is covalently linked to another suitable therapeutic agent, or a composition comprising a humanized TDP-43 specific binding molecule combined with a pharmaceutically acceptable carrier and/or excipient and/or diluent.

In some embodiments the humanized TDP-43 specific binding molecule is part of a detection and/or diagnostic kit comprising a humanized TDP-43 specific binding molecule, or an immunoconjugate wherein the humanized TDP-43 specific binding molecule is covalently linked to another suitable therapeutic agent, or a composition comprising a humanized TDP-43 specific binding molecule.

Kits containing the humanized binding molecules of the invention are also provided. In particular, such kits may be useful for performing the diagnostic methods of the invention (which include classification, monitoring and therapy selection methods). Thus, a kit for diagnosis of a disease, disorder and/or abnormality associated with TDP-43, in particular associated with TDP-43 aggregates, or a TDP-43 proteinopathy, or for use in a method of the invention is provided comprising a humanized TDP-43 specific binding molecule of the invention. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed. Suitably labelled humanized TDP-43 binding molecules of the invention are also provided, which may be included in such kits.

In some embodiments the humanized TDP-43 specific binding molecule is used in an immunodiagnostic method for use in the prevention, diagnosis or treatment of a TDP-43 proteinopathy.

In some embodiments the humanized TDP-43 specific binding molecule, or an immunoconjugate wherein the humanized TDP-43 specific binding molecule is covalently linked to another suitable therapeutic agent, or a composition comprising a humanized TDP-43 specific binding molecule, is administered to a subject in need thereof or is used to diagnose, prevent, alleviate or treat a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS), Alzheimer's disease (AD), Parkinson's disease (PD), Chronic Traumatic Encephalopathy (CTE), limbic-predominant age-related TDP-43 encephalopathy (LATE).

In some embodiments the humanized TDP-43 specific binding molecule, or an immunoconjugate wherein the humanized TDP-43 specific binding molecule is covalently linked to another suitable therapeutic agent, or a composition comprising a humanized TDP-43 specific binding molecule, is administered to a subject in need thereof or is used in a method for diagnosing or monitoring a disease, disorder and/or abnormality associated with TDP-43, in particular associated with TDP-43 aggregates, or TDP-43 proteinopathy selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In other embodiment, the invention relates to any methods for detecting, diagnosing or monitoring a disease, disorder and/or abnormality associated with TDP-43, in particular associated with TDP-43 aggregates, or TDP-43 proteinopathy that is selected from frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

Preferably, the disease, disorder and/or abnormality associated with TDP-43, in particular associated with TDP-43 aggregates, or TDP-43 proteinopathies selected from amyotrophic lateral sclerosis (ALS), Alzheimer's disease (AD) and Frontotemporal dementia (FTD). More preferably, the disease, disorder and/or abnormality associated with TDP-43, in particular associated with TDP-43 aggregates, or TDP-43 proteinopathy is amyotrophic lateral sclerosis (ALS). More preferably, the disease, disorder and/or abnormality associated with TDP-43, in particular associated with TDP-43 aggregates, or TDP-43 proteinopathy is Alzheimer's disease (AD). More preferably, the disease, disorder and/or abnormality associated with TDP-43, in particular associated with TDP-43 aggregates, or TDP-43 proteinopathy is Frontotemporal dementia (FTD).

In some embodiments, the humanized TDP-43 specific binding molecule is used in a method for diagnosing presymptomatic disease or for monitoring disease progression and therapeutic efficacy, or for predicting responsiveness, or for selecting subjects which are likely to respond to the treatment with a humanized TDP-43 specific binding molecule. Said method is preferably performed using a sample of human blood or urine. Most preferably the method involves an ELISA-based or surface adapted assay.

In some embodiments the humanized TDP-43 specific binding molecule is used in a method wherein a humanized TDP-43 specific binding molecule of the present invention is contacted with a sample (e.g., blood, urine, cerebrospinal fluid, interstitial fluid (ISF), or brain tissue) to detect, diagnose or monitor frontotemporal degeneration (FTD) or amyotrophic lateral sclerosis (ALS), Alzheimer's disease (AD), Chronic Traumatic Encephalopathy (CTE), Perry syndrome, limbic-predominant age-related TDP-43 encephalopathy (LATE) and/or Parkinson's disease (PD).

In some embodiments, the humanized TDP-43 specific binding molecule is used in a method wherein a humanized TDP-43 specific binding molecule of the present invention is contacted with a sample (e.g., blood, urine, cerebrospinal fluid, interstitial fluid (ISF), or brain tissue) to detect, diagnose or monitor a disease selected from Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

In some embodiments, the humanized TDP-43 specific binding molecule, or an immunoconjugate wherein the humanized TDP-43 specific binding molecule is covalently linked to another suitable therapeutic agent, or a composition comprising a humanized TDP-43 specific binding molecule, is administered to a subject in need thereof or is used for preventing, alleviating or treating a disease, disorder and/or abnormality associated with TDP-43, in particular associated with TDP-43 aggregates, or TDP-43 proteinopathies, or frontotemporal degeneration (FTD) or amyotrophic lateral sclerosis (ALS), Alzheimer's disease (AD, including sporadic and familial forms of AD), Chronic Traumatic Encephalopathy (CTE), Perry syndrome and limbic-predominant age-related TDP-43 encephalopathy (LATE) and/or Parkinson's disease (PD).

In some embodiments, the humanized TDP-43 specific binding molecule, or an immunoconjugate wherein the humanized TDP-43 specific binding molecule is covalently linked to another suitable therapeutic agent, or a composition comprising a humanized TDP-43 specific binding molecule, is administered to a subject in need thereof or is used for treating a disease selected from: Frontotemporal dementia (FTD, such as sporadic or familial with or without motor-neuron disease (MND), with progranulin (GRN) mutation, with C9orf72 mutations, with TARDBP mutation, with valosin-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD). Preferably said disease treatment helps to retain or increase mental recognition and or reduces the level of TDP-43 aggregates in the brain.

In some embodiments the humanized TDP-43 specific binding molecule, or an immunoconjugate wherein the humanized TDP-43 specific binding molecule is covalently linked to another suitable therapeutic agent, or a composition comprising a humanized TDP-43 specific binding molecule, is administered to a subject in need thereof or is used for manufacturing a medicament for treating a disease, disorder and/or abnormality associated with TDP-43, in particular associated with TDP-43 aggregates, or TDP-43 proteinopathies or frontotemporal degeneration (FTD) or amyotrophic lateral sclerosis (ALS), Alzheimer's disease (AD, including sporadic and familial forms of AD), Chronic Traumatic Encephalopathy (CTE), Perry syndrome and limbic-predominant age-related TDP-43 encephalopathy (LATE), and/or Parkinson's disease (PD).

Pharmaceutical formulations of a humanized anti-TDP-43 antibody (the preferred type of TDP-43 specific binding molecule) or immunoconjugate as described herein are prepared by mixing such humanized antibody or immunoconjugate having the desired degree of purity with one or more optional pharmaceutically acceptable carriers and/or excipients and/or diluents (Remington's Pharmaceutical Sciences 16th edition, Osol, A. Ed. (1980)). Typically, the antibody or fragment therefor is prepared as a lyophilized formulation or aqueous solution. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and lanolin. Diluents may be buffers. They may comprise a salt selected from the group consisting of phosphate, acetate, citrate, succinate and tartrate, and/or wherein the buffer comprises histidine, glycine, TRIS glycine, Tris, or mixtures thereof. It is further envisaged in the context of the present invention that the diluent is a buffer selected from the group consisting of potassium phosphate, acetic acid/sodium acetate, citric acid/sodium citrate, succinic acid/sodium succinate, tartaric acid/sodium tartrate, and histidine/histidine HCl or mixtures thereof.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules. The formulations to be used for in vivo administration are generally sterile. Sterility may be readily accomplished, e.g., by filtration through sterile filtration membranes.

Any of the humanized antigen-binding molecules, humanized anti-TDP-43 antibodies or immunoconjugates provided herein may be used in methods, e.g., therapeutic methods.

In another aspect, a humanized anti-TDP-43 antibody (the preferred type of humanized TDP-43 specific binding molecule) or immunoconjugate for use as a medicament is provided. In further aspects, an anti-misfolded humanized TDP-43 antibody (the preferred type of humanized TDP-43 specific binding molecule) or immunoconjugate for use in a method of treatment is provided. In certain embodiments, a humanized anti-TDP-43 antibody (the preferred type of humanized TDP-43 specific binding molecule) or immunoconjugate for use in the prevention, diagnosis and/or treatment of a TDP-43 proteinopathy is provided. In a preferred embodiment of the invention, a humanized anti-TDP-43 antibody (the preferred type of humanized TDP-43 specific binding molecule) or immunoconjugate is provided for use in the prevention, diagnosis and/or treatment of a disease, disorder and/or abnormality associated with TDP-43, in particular associated with TDP-43 aggregates, or TDP-43 proteinopathy including but not limited to frontotemporal dementia (FTD), amyotrophic lateral sclerosis (ALS), Alzheimer's disease (AD), Parkinson's disease (PD), Chronic Traumatic Encephalopathy (CTE), and/or limbic-predominant age-related TDP-43 encephalopathy (LATE).

In a further aspect, the invention provides for the use of a humanized anti-TDP-43 antibody (the preferred type of humanized TDP-43 specific binding molecule) or immunoconjugate in the manufacture or preparation of a medicament. In one such embodiment, the method further comprises administering to the individual an effective amount of at least one additional therapeutic agent, e.g., as described below.

A “subject” or an “individual” according to any of the embodiments may be an animal, a mammal, preferably a human.

In a further aspect, the invention provides pharmaceutical formulations comprising any of the humanized anti-TDP-43 antibodies (the preferred type of humanized TDP-43 specific binding molecule) or immunoconjugate provided herein, e.g., for use in any of the therapeutic methods. In one embodiment, a pharmaceutical formulation comprises any of the humanized anti-TDP-43 antibodies (the preferred type of humanized TDP-43 specific binding molecule) or immunoconjugates provided herein and a pharmaceutically acceptable carrier and/or excipients and/or diluents (as discussed elsewhere herein). In another embodiment, a pharmaceutical formulation comprises any of the humanized anti-TDP-43 antibodies (the preferred type of humanized TDP-43 specific binding molecule) or immunoconjugates provided herein and at least one additional therapeutic agent, e.g., as described below.

Humanized antibodies or immunoconjugates of the invention can be used either alone or in combination with other agents in a therapy. For instance, a humanized antibody (the preferred type of humanized TDP-43 specific binding molecule) or immunoconjugate of the invention may be co-administered with at least one additional therapeutic agent targeting alpha-synuclein, BACE1, Tau, beta-amyloid, TDP-43 or a neuroinflammation protein.

For instance, an humanized antibody (the preferred type of humanized TDP-43 specific binding molecule) or immunoconjugate of the invention may be co-administered with at least one additional therapeutic agent which is selected from, but not limited to, neurological drugs, anti-amyloid beta antibodies, anti-Tau antibodies, Tau aggregation inhibitors (including small molecules), beta-amyloid aggregation inhibitors (including small molecules), anti-BACE1 antibodies, BACE1 inhibitors, anti-alpha-synuclein inhibitors, anti-alpha-synuclein antibodies and neuroinflammation inhibitors.

Such combination therapies noted above encompass combined administration (where two or more therapeutic agents are included in the same or separate formulations), and separate administration, in which case, administration of the humanized antibody (the preferred type of humanized TDP-43 specific binding molecule) or immunoconjugate of the invention can occur prior to, simultaneously, and/or following, administration of the additional therapeutic agent and/or adjuvant. Humanized antibodies (the preferred type of humanized TDP-43 specific binding molecule) or immunoconjugates of the invention can also be used in combination with radiation therapy.

A humanized antibody (the preferred type of humanized TDP-43 specific binding molecule) or immunoconjugate of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Humanized antibodies (the preferred type of humanized TDP-43 specific binding molecule) or immunoconjugates of the invention would be formulated, dosed, and administered in a fashion consistent with good medical practice. Factors for consideration in this context include the particular disease, disorder and/or abnormality associated with TDP-43, in particular associated with TDP-43 aggregates, or TDP-43 proteinopathy being treated, the particular mammal being treated, the clinical condition of the individual subject, the cause of the disease, a disorder and/or abnormality associated with TDP-43, in particular associated with TDP-43 aggregates, or TDP-43 proteinopathy, the site of delivery of the agent, the method of administration, the scheduling of administration, and other factors known to medical practitioners. The humanized antibody or immunoconjugate need not be, but is optionally formulated with one or more agents currently used to prevent or treat the disease, disorder and/or abnormality associated with TDP-43, in particular associated with TDP-43 aggregates, or TDP-43 proteinopathy in question. The effective amount of such other agents depends on the amount of humanized antibody or immunoconjugate present in the formulation, the type of disease, disorder and/or abnormality associated with TDP-43, in particular associated with TDP-43 aggregates or TDP-43 proteinopathy or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an humanized antibody (the preferred type of humanized TDP-43 specific binding molecule) or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the subject's clinical history and response to the antibody or immunoconjugate, and the discretion of the attending physician. The humanized antibody (the preferred type of humanized TDP-43 specific binding molecule) or immunoconjugate is suitably administered to the subject at one time or over a series of treatments. Depending on the type and severity of the disease, about 1 μg/kg to 15 mg/kg (e.g. 0.1 mg/kg-10 mg/kg) of humanized antibody (the preferred type of humanized TDP-43 specific binding molecule) or immunoconjugate can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the humanized antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the subject receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using both an immunoconjugate of the invention and a humanized anti-TDP-43 antibody (the preferred type of humanized TDP-43 specific binding molecule).

In another aspect of the invention, an article of manufacture containing materials useful for the treatment, prevention and/or diagnosis of the diseases, disorders or abnormalities associated with TDP-43, in particular associated with TDP-43 aggregates, or TDP-43 proteinopathy, described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder and/or abnormality associated with TDP-43, in particular associated with TDP-43 aggregates, or TDP-43 proteinopathy, and may have a sterile access port (for example the container may be an intravenous solution bag or a vial having a stopper pierceable by a hypodermic injection needle). At least one active agent in the composition is a humanized antibody or immunoconjugate of the invention. The label or package insert indicates that the composition is used for treating the condition of choice.

Moreover, the article of manufacture may comprise (a) a first container with a composition contained therein, wherein the composition comprises a humanized antibody (the preferred type of humanized TDP-43 specific binding molecule) or immunoconjugate of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further embodiment, the invention relates to a method of retaining or increasing cognitive memory capacity, movement and language function or preventing and/or slowing decline of cognitive memory capacity, movement and language function in a subject, comprising administering the humanized binding molecule of the invention, the immunoconjugate of the invention, the composition of the invention or the pharmaceutical composition of the invention.

In a further embodiment, the invention relates to a method of reducing the level of TDP-43, comprising administering the humanized binding molecule of the invention, the immunoconjugate of the invention, the composition of the invention or the pharmaceutical composition of the invention.

The methods of the invention may comprise administering at least one additional therapy, preferably wherein the additional therapy is selected from, but not limited to, antibodies or small molecules targeting alpha-synuclein, BACE1, tau, beta-amyloid, TDP-43 or a neuroinflammation protein, in particular neurological drugs, anti-beta-amyloid antibodies, anti-Tau antibodies, Tau aggregation inhibitors, beta-amyloid aggregation inhibitors, anti-BACE1 antibodies, BACE1 inhibitors, anti-alpha-synuclein antibodies and neuroinflammation inhibitors.

The invention furthermore relates to a method of detecting TDP-43, comprising contacting a sample with the humanized binding molecule of the invention, preferably a humanized antibody of the invention wherein the sample is a brain sample, a cerebrospinal fluid sample, an interstitial fluid (ISF) sample, urine sample or a blood sample.

In further embodiment, the invention relates to a method of detecting and/or measuring the level of TDP-43, comprising contacting a sample with the humanized binding molecule of the invention, preferably a humanized antibody of the invention, using Single Molecule Array (SIMOA®) technology, wherein the sample is a human blood sample, a cerebrospinal fluid sample (CSF), an interstitial fluid (ISF) sample or a urine sample, preferably a CSF sample.

In certain embodiments, the humanized TDP-43 binding molecule, in particular humanized TDP-43 antibody and fragment thereof as provided herein has a dissociation constant (KD) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M), in particular with respect to binding TDP-43, in particular soluble TDP-43. For example, the humanized TDP-43 binding molecules of the invention may have a KD for soluble full-length TDP-43 of 2 nM or less, in specific embodiments 1 nM or less, in more specific embodiments a KD for soluble full-length TDP-43 of 700 pM or less, preferably 250 pM or less. This is demonstrated for humanized TDP-43 binding molecules of the invention in Example 3 with reference to Table 8. In one embodiment, binding affinity to full length (FL) TDP-43 may be evaluated by determining the dissociation constants (KD) using surface plasmon resonance (SPR; Biacore 8K, GE Healthcare Life Sciences). Reference may be made to Examples 3 for a detailed description of suitable SPR methods that may be employed.

The humanized TDP-43 binding molecules of the invention may have a KD for TP-51 peptide of 15 nM or less, in specific embodiments 10 nM or less, such as 4 nM or less and preferably 2 nM or less. This is also demonstrated for humanized TDP-43 binding molecules of the invention in Example 3 with reference to Table 8.

Thus, the humanized TDP-43 binding molecules of the invention may have a KD for soluble full-length TDP-43 of 575 pM or less and a KD for TP-51 peptide of 2.2 nM or less, a KD for soluble full-length TDP-43 of 575 pM or less and a KD for TP-51 peptide of 1.6 nM or less, a KD for soluble full-length TDP-43 of 550 pM or less and a KD for TP-51 peptide of 2.2 nM or less, preferably a KD for soluble full-length TDP-43 of 550 pM or less and a KD for TP-51 peptide of 2 nM or less, more preferably a KD for soluble full-length TDP-43 of 250 pM or less and a KD for TP-51 peptide of 1.6 nM or less, even more preferably a KD for soluble full-length TDP-43 of 150 pM or less and a KD for TP-51 peptide of 1.0 nM or less, still more preferably a KD for soluble full-length TDP-43 of 130 pM or less and a KD for TP-51 peptide of 0.8 nM or less.

The humanized TDP-43 binding molecule of the invention may neutralize seeding-competent TDP-43. Neutralization of seeding component TDP_43 may be evaluated by a real-time quaking-induced conversion (RT-QuIC) assay. Reference may be made to Example 11 for a detailed description of suitable RT-QuIC assay that may be employed. By “seeding-competent TDP-43” is meant pathological TDP-43 able to induce aggregation of physiological (i.e. non-pathological TDP-43).

The humanized TDP-43 binding molecule of the invention may potentiate TDP-43 clearance. Potentiation of TDP-43 clearance may be evaluated by an in vitro assay using THP-1 cells. Reference may be made to Example 12 for a detailed description of a suitable assay using THP-1 cells that may be employed. In general terms, TDP-43 clearance is potentiated by the antibodies of the invention by binding to pathological TDP-43 and forming an immune complex that may be cleared by antibody-dependent cellular phagocytosis (ADCP). ADCP is mediated by the interaction of antibody Fc fragment with Fc receptors, such as Fc gamma receptors, expressed at the surface of innate immune cells, such as microglia or dendritic cells. Fc mediated functions may be modulated to achieve the desired effect by modifying the Fc portion of antibodies.

In some embodiments, the humanized TDP-43 binding molecule of the invention has an EC50 of less than 250 pM, preferably less than or equal to 237 pM for TDP-43 in human CSF. The humanized TDP-43 binding molecule may be hACI-7069-633B12-Ab1_H33L27. Reference can be made to Example 14 for how to measure EC50.

### BRIEF DESCRIPTION OF THE FIGURES

FIG. 1. Inhibition of TDP-43 aggregation in vitro by hACI-7069-633B12-Ab1_H33L27, hACI-7069-633B12-Ab1_H32L27 and hACI-7069-633B12-Ab1_H32L26. Briefly, recombinant TDP-43 de novo aggregation upon cleavage from MBP-tag was analysed in the presence of either hACI-7069-633B12-Ab1_H33L27, hACI-7069-633B12-Ab1_H32L27 and hACI-7069-633B12-Ab1_H32L26 and compared to the chimeric antibody cACI-7069-633B12-Ab1 or an isotype control over time. The percentage of aggregated TDP-43 at 1.5h was normalized to the Isotype Control condition and showed a strong inhibition of aggregation with cACI-7069-633B12-Ab1, hACI-7069-633B12-Ab1_H33L27, hACI-7069-633B12-Ab1_H32L27 and hACI-7069-633B12-Ab1_H32L26. A one-way ANOVA followed by a Dunnett's post-hoc test for multiple comparisons was used. ****p<0.0001.

FIG. 2. Pharmacokinetic profiles in homozygous Tg32 mice serum following a single IV bolus administration of either hACI-7069-633B12-Ab1_H33L27 (circles) or hACI-7069-633B12-Ab1_H32L27 (squares) at 40 mg/kg. 21 male mice were used per compound with 3 animals per time point. The total duration of the study was 6 weeks (1008h).

FIG. 3. Pharmacokinetic profiles in cynomolgus monkey serum following a single IV bolus administration of either hACI-7069-633B12-Ab1_H33L27 (A) or hACI-7069-633B12-Ab1_H32L27 (B) at 40 mg/kg. 4 male cynomolgus monkeys were used per compound for a study duration of 8 weeks (1344h).

FIG. 4. Pharmacodynamic profiles of TDP-43 in cynomolgus monkey serum following a single IV bolus administration of either hACI-7069-633B12-Ab1_H33L27 (A) or hACI-7069-633B12-Ab1_H32L27 (B) at 40 mg/kg. Target engagement of both hACI-7069-633B12-Ab1_H33L27 (A) and hACI-7069-633B12-Ab1_H32L27 (B) in serum was evaluated.

FIG. 5. Real-Time Quaking-induced Conversion (RT-QuIC) aggregation kinetics of a synthetic TDP-43 peptide (TP-51) in presence of cerebrospinal fluid (CSF) from either sporadic ALS (FIG. 5A) or healthy control (FIG. 5B) donors. The aggregation and fibrils formation were quantified using Thioflavin T (ThT) fluorescence.

FIG. 6. pHrodo™-TDP-43 aggregates uptake by THP-1 cells is significantly accelerated in the presence of either hACI-7069-633B12-Ab1_H33L27 or the chimeric antibody cACI-7069-633B12-Ab1 but not in presence of an isotype antibody control. (A) shows pHrodo™ fluorescence intensity (Y axis) measured hourly over 24h (X axis) for aggregate TDP-43, aggregate TDP-43 with isotype control, aggregate TDP-43 with cACI-7069-633B12-Ab1 and aggregate TDP-43 with hACI-7069-633B12-Ab1_H33L27 incubated cells. (B) shows the difference in fluorescence intensity normalized to cell confluency for the 10h time point for the aggregate TDP-43, aggregate TDP-43 with isotype control, aggregate TDP-43 with cACI-7069-633B12-Ab1 and aggregate TDP-43 with hACI-7069-633B12-Ab1_H33L27 incubated cells.

FIG. 7. (A) Representative image of TDP-43 immunoblots from FTLD-TDP brain-derived extracts following immunodepletion by either hACI-7069-633B12-Ab1_H33L27 (lane 1), cACI-7069-633B12-Ab1 (lane 2) or the isotype antibody control (lane 3). (B) shows the quantitative analyses the signals obtained for total TDP-43 (B—top panel) or C-terminal fragments of TDP-43 (i.e., lower than 25 kDa, CTF) (B—bottom panel) normalized to the corresponding signal in the input.

FIG. 8. Graphical representation of TDP-43 target engagement of hACI-7069-633B12-Ab1_H33L27 in a human CSF sample.

FIG. 9. Serum exposure of hACI-7069-633B12-Ab1_H33L27 in cynomolgus monkey following once weekly IV infusion for four weeks at doses of 40, 120 and 360 mg/kg.

FIG. 10. (A) Pharmacokinetic profiles in homozygous Tg32 mice serum following a single IV bolus administration of hACI-7069-633B12-Ab1_H33L27, hACI-7069-633B12-Ab1_H19L18 or cACI-7069-633B12-Ab1 at 40 mg/kg. (B) Pharmacokinetic profiles in cynomolgus monkey serum following a single IV bolus administration of hACI-7069-633B12-Ab1_H33L27, hACI-7069-633B12-Ab1_H19L18 or cACI-7069-633B12-Ab1 at 40 mg/kg.

### EXAMPLES

### Example 1. Humanization of Anti-Human TDP-43 Mouse Monoclonal Antibody

Design of the humanized variable regions

The variable domain of ACI-7069-633B12-Ab1 has a theoretical pI of 8.5 and a net charge of +3.1 at physiological pH. It has been shown that in some cases the high net positive charge of antibody was involved in the fast clearance of antibody in vivo (Igawa et al, PEDS. 2010; vol. 23 no. 5 pp. 385-392, Bumbaca et al, JBC, VOL. 290, NO. 50, pp. 29732-29741, 2015). To optimize the charge profile of the molecule, a pair of acceptor framework with low pI was selected to graft ACI-7069-633B12-Ab1 CDRs (CDRs as described in WO2020/234473). First, heavy and light chain frameworks were ranked based on sequence identity to the murine heavy and light chain V regions (SEQ ID NO: 20 and 24, respectively). Databases of human and mouse germline variable genes such as the IMGT database (Ehren mann, F et al, (2010) Nucl. Acids Res., 38 (S1): D301-D307) or IgBlast (Ye J. et al, (2013), Nucleic Acids Res. 2013 July; 41 (Web Server issue): W34-W40) may be used to identify such germline variable genes. Second, the theoretical pI of each individual chain was calculated using the web server Isoelectric Point Calculator 2.0 (Kozlowski L P et al, Nucleic Acids Res. 49 (W1): W285-W292.) The average pI value from all available models was taken in consideration as well as the net charge calculated at pH 7.4.

For the heavy chain variable domain, framework IGHV1-69-2, IGHV1-24, IGHV5-51 or IGHV3-43 could be used as acceptor framework while for the light chain variable domain IGKV2-24, IGKV2-40, IGKV2D-40, IGKV2D-28, IGKV4-1 or IGKV2-28 could be used as acceptor framework. All the listed frameworks have a theoretical pI and net charge equal or lower than the variable domains of ACI-7069-633B12-Ab1.

Whilst a marginal reduction in pI was observed with human frameworks such as IGHV 1-24 and IGKV2-40, the pair formed by IGHV1-69-2 and IGKV2-28 reduced substantially the Fv net charge and provided a balanced charge distribution. The use of IGHV1-69-2 as human acceptor framework disrupts a large patch of positive charges by replacing murine positively charged residues with substitutions K64Q, R82aS and K73T. To a lesser degree, the use of germline IGKV2-28 for VL, contributes to the charge reduction with substitution K45Q in framework 2, which removes the positive charge while maintaining the hydrophilicity of the molecule. From this analysis, new humanized variants were generated (Table 3) based on IGHV1-69-2 and IGKV2-28 human frameworks.

IGHV1-69-2 and IGKV2-28 were chosen as acceptor framework for heavy and light chain variable domain respectively. IGHV1-69-2 has 64.9% sequence identity with ACI-7069-633B12-Ab1 VH while having a pI of 4.3 and a net charge of −6.9 at physiological pH. IGKV2-28 has 72% sequence identity with ACI-7069-633B12-Ab1 VH while having a pI of 4.7 and a net charge of −2.9 at physiological pH. IGHJ2*01 and IGKJ2*02 were used as J junction gene for heavy and light chain variable domain respectively.

As starting point for the humanization process, murine CDRs were grafted on human acceptor frameworks for both VH and VL regions. To accommodate CDRs on the human acceptor framework, key positions were modified by substituting human to mouse residues.

To identify residues that may most greatly impact CDR conformation and/or VH/VL orientation, a 3D model for the human-mouse hybrid VH-VL pair was generated by homology modelling using the Abodybuilder server (8). Model analysis allowed the selection of a subset of amino acid positions including the positions listed in Table 2. Numbering is according to the Kabat numbering system.

TABLE 2
Backmutations introduced in the human framework
(Kabat numbering) of ACI-7069-633B12-Ab1

| | Position | Mouse | Human |
|---|---|---|---|
| Variable domain | (Kabat) | residue | residue |
| VH | 24 | T | V |
| VH | 27 | F | Y |
| VH | 48 | I | M |
| VH | 64 | K | Q |
| VH | 71 | V | A |
| VH | 73 | K | T |
| VH | 94 | R | T |
| VL | 2 | V | I |
| VL | 36 | L | Y |
| VL | 45 | K | Q |
| VL | 46 | R | L |
| VL | 57 | R | G |

Potential post translational modification sites were identified within AC3-7069-63312-Ab1 CDR sequences. In the variable heavy chain, N53, N54 and G55 were identified as two deamidation sites. In the variable light chain, an isomerization site was recognized at position D28 and G29, whereas an oxidation site was identified at position W89 (according to Kabat numbering system). In some constructs, point mutations including N53Q and/or G55A were introduced in the VH region whereas G29A and/or W89F were introduced in the VL region to remove the post translational modification site in CDR Li and L3.

The reduction in p1 and charge is exemplified in Table 3.

TABLE 3
Theoretical pI and net charge at pH7.4 of the variable
domain of ACI-7069-633B12-Ab1 humanized
variants calculated using the webserver Isoelectric point
calculator 2.0 (IPC 2.0 webserver)

| | | Net charge at |
|---|---|---|
| Antibody code | Fv pI | pH 7.4 |
| ACI-7069-633B12-Ab1 | 8.5 | 3.1 |
| hACI-7069-633B12-Ab1_H27L23 | 7.4 | −0.9 |
| hACI-7069-633B12-Ab1_H28L22 | 7.7 | 0.1 |
| hACI-7069-633B12-Ab1_H28L24 | 7.4 | −0.9 |
| hACI-7069-633B12-Ab1_H32L24 | 6.9 | −1.9 |
| hACI-7069-633B12-Ab1_H32L26 | 6.9 | −1.9 |
| hACI-7069-633B12-Ab1_H33L24 | 6.9 | −1.9 |
| hACI-7069-633B12-Ab1_H33L26 | 6.9 | −1.9 |
| hACI-7069-633B12-Ab1_H32L27 | 6.9 | −1.9 |
| hACI-7069-633B12-Ab1_H33L27 | 6.9 | −1.9 |

Backmutations from Table 2 were combined to generate the sequences respectively listed in Tables 4 and 6. The corresponding nucleic acid sequences encoding the humanized TDP-43 binding molecules are shown in Tables 5 and 7.

TABLE 4
Amino acid sequence of the heavy chains (VH) and their CDRs

| Chain | Heavy chain | CDRH1 | CDRH2 | CDRH3 |
|---|---|---|---|---|
| ACI-7069-633B12- | EVQLQQSGPELVKPGASVKISCKTSGFTFTEYSMHWVK | EYSMH | GINPNNGGTSYNQKF | ES |
| Ab1_VH | QSHGKSLEWIGGINPNNGGTSYNQKFKGKATLTVDKSS | (SEQ | KG | |
| | STAYMELRSLTSEDSAVYYCARESWGQGTTLTVSS | ID NO: | (SEQ ID NO: 22) | |
| | (SEQ ID NO: 20) | 21) | | |
| hACI-7069-633B12- | EVQLVQSGAEVKKPGATVKISCKTSGFTFTEYSMHWV | EYSMH | GINPNNGGTSYNQKF | ES |
| Ab1_H24 | QQAPGKGLEWIGGINPNNGGTSYNQKFKGRVTITVDKS | (SEQ | KG | |
| | TDTAYMELSSLRSEDTAVYYCARESWGQGTTVTVSS | ID NO: | (SEQ ID NO: 22) | |
| | (SEQ ID NO: 30) | 21) | | |
| hACI-7069-633B12- | EVQLVQSGAEVKKPGATVKISCKTSGFTFTEYSMHWV | EYSMH | GINPNNGGTSYNQKF | ES |
| Ab1_H25 | QQAPGKGLEWIGGINPNNGGTSYNQKFKGRVTITVDTS | (SEQ | KG | |
| | TDTAYMELSSLRSEDTAVYYCARESWGQGTTVTVSS | ID NO: | (SEQ ID NO: 22) | |
| | (SEQ ID NO: 40) | 21) | | |
| hACI-7069-633B12- | EVQLVQSGAEVKKPGATVKISCKTSGFTFTEYSMHWV | EYSMH | GINPNNGGTSYNQKF | ES |
| Ab1_H26 | QQAPGKGLEWMGGINPNNGGTSYNQKFKGRVTITVDT | (SEQ | KG | |
| | STDTAYMELSSLRSEDTAVYYCARESWGQGTTVTVSS | ID NO: | (SEQ ID NO: 22) | |
| | (SEQ ID NO: 50) | 21) | | |
| hACI-7069-633B12- | EVQLVQSGAEVKKPGATVKISCKVSGFTFTEYSMHWV | EYSMH | GINPNNGGTSYNQKF | ES |
| Ab1_H27 | QQAPGKGLEWMGGINPNNGGTSYNQKFKGRVTITVDT | (SEQ | KG | |
| | STDTAYMELSSLRSEDTAVYYCARESWGQGTTVTVSS | ID NO: | (SEQ ID NO: 22) | |
| | (SEQ ID NO: 60) | 21) | | |
| hACI-7069-633B12- | EVQLVQSGAEVKKPGATVKISCKVSGFTFTEYSMHWV | EYSMH | GINPNNGGTSYNQKF | ES |
| Ab1_H28 | QQAPGKGLEWMGGINPNNGGTSYNQKFKGRVTITADT | (SEQ | KG | |
| | STDTAYMELSSLRSEDTAVYYCARESWGQGTTVTVSS | ID NO: | (SEQ ID NO: 22) | |
| | (SEQ ID NO: 70) | 21) | | |
| hACI-7069-633B12- | EVQLVQSGAEVKKPGATVKISCKVSGFTFTEYSMHWV | EYSMH | GINPNQGGTSYNQKF | ES |
| Ab1_H29 | QQAPGKGLEWMGGINPNQGGTSYNQKFKGRVTITADT | (SEQ | KG | |
| | STDTAYMELSSLRSEDTAVYYCARESWGQGTTVTVSS | ID NO: | (SEQ ID NO: 82) | |
| | (SEQ ID NO: 80) | 21) | | |
| hACI-7069-633B12- | EVQLVQSGAEVKKPGATVKISCKVSGFTFTEYSMHWV | EYSMH | GINPQNAGTSYNQKF | ES |
| Ab1_H30 | QQAPGKGLEWMGGINPQNAGTSYNQKFKGRVTITADT | (SEQ | KG | |
| | STDTAYMELSSLRSEDTAVYYCARESWGQGTTVTVSS | ID NO: | (SEQ ID NO: 92) | |
| | (SEQ ID NO: 90) | 21) | | |
| hACI-7069-633B12- | EVQLVQSGAEVKKPGATVKISCKVSGFTFTEYSMHWV | EYSMH | GINPNQGGTSYNQKF | ES |
| Ab1_H31 | QQAPGKGLEWMGGINPNQGGTSYNQKFQGRVTITADT | (SEQ | QG | |
| | STDTAYMELSSLRSEDTAVYYCARESWGQGTTVTVSS | ID NO: | (SEQ ID NO: 102) | |
| | (SEQ ID NO: 100) | 21) | | |
| hACI-7069-633B12- | EVQLVQSGAEVKKPGATVKISCKVSGFTFTEYSMHWV | EYSMH | GINPQNAGTSYNQKF | ES |
| Ab1_H32 | QQAPGKGLEWMGGINPQNAGTSYNQKFQGRVTITADT | (SEQ | QG | |
| | STDTAYMELSSLRSEDTAVYYCARESWGQGTTVTVSS | ID NO: | (SEQ ID NO: 112) | |
| | (SEQ ID NO: 110) | 21) | | |
| hACI-7069-633B12- | EVQLVQSGAEVKKPGATVKISCKVSGFTFTEYSMHWV | EYSMH | GINPNNGGTSYNQKF | ES |
| Ab1_H33 | QQAPGKGLEWMGGINPNNGGTSYNQKFQGRVTITADT | (SEQ | QG | |
| | STDTAYMELSSLRSEDTAVYYCARESWGQGTTVTVSS | ID NO: | (SEQ ID NO: 122) | |
| | (SEQ ID NO: 120) | 21) | | |

TABLE 5
DNA sequence of the humanized heavy chain variable domains (VH)

| Chain | Heavy chain |
|---|---|
| *ACI-7069-633B12- | GAGGTGCAGCTGCAGCAGAGCGGCCCCGAACTGGTGAAGCCCGGCGCCTCCGTGAAGATCAGCTGCAAGAC |
| Ab1_VH | CAGCGGCTTCACCTTCACCGAGTACAGCATGCACTGGGTGAAGCAGTCCCACGGCAAGTCTCTGGAGTGGAT |
| | CGGCGGCATCAACCCCAACAACGGCGGCACCAGCTACAACCAGAAGTTCAAGGGCAAGGCCACACTGACAC |
| | TGGACAAGAGCAGCAGCACCGCCTACATGGAGCTGAGATCTCTGACCAGCGAGGACTCCGCCGTGTACTAC |
| | TGCGCCAGAGAGAGCTGGGGCCAAGGCACCACACTGACCGTGAGCAGC (SEQ ID NO: 28) |
| hACI-7069-633B12- | GAAGTGCAGCTGGTGCAGAGCGGCGCGGAAGTGAAAAAACCGGGCGCGACCGTGAAAATTAGCTGCAAAA |
| Ab1_H24 | CCAGCGGCTTTACCTTTACCGAATATAGCATGCATTGGGTGCAGCAGGCGCCGGGCAAAGGCCTGGAATGG |
| | ATTGGCGGCATTAACCCGAACAACGGCGGCACCAGCTATAACCAGAAATTTAAAGGCCGTGTGACCATTAC |
| | CGTGGATAAAAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 38) |
| hACI-7069-633B12- | GAAGTGCAGCTGGTGCAGAGCGGCGCGGAAGTGAAAAAACCGGGCGCGACCGTGAAAATTAGCTGCAAAA |
| Ab1_H25 | CCAGCGGCTTTACCTTTACCGAATATAGCATGCATTGGGTGCAGCAGGCGCCGGGCAAAGGCCTGGAATGG |
| | ATTGGCGGCATTAACCCGAACAACGGCGGCACCAGCTATAACCAGAAATTTAAAGGCCGTGTGACCATTAC |
| | CGTGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 48) |
| hACI-7069-633B12- | GAAGTGCAGCTGGTGCAGAGCGGCGCGGAAGTGAAAAAACCGGGCGCGACCGTGAAAATTAGCTGCAAAA |
| Ab1_H26 | CCAGCGGCTTTACCTTTACCGAATATAGCATGCATTGGGTGCAGCAGGCGCCGGGCAAAGGCCTGGAATGG |
| | ATGGGCGGCATTAACCCGAACAACGGCGGCACCAGCTATAACCAGAAATTTAAAGGCCGTGTGACCATTAC |
| | CGTGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 58) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H27 | TGAGCGGCTTTACCTTTACCGAATATAGCATGCATTGGGTGCAGCAGGCGCCGGGCAAAGGCCTGGAATGG |
| | ATGGGCGGCATTAACCCGAACAACGGCGGCACCAGCTATAACCAGAAATTTAAAGGCCGTGTGACCATTAC |
| | CGTGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 68) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H28 | TGAGCGGCTTTACCTTTACCGAATATAGCATGCATTGGGTGCAGCAGGCGCCGGGCAAAGGCCTGGAATGG |
| | ATGGGCGGCATTAACCCGAACAACGGCGGCACCAGCTATAACCAGAAATTTAAAGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 78) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H29 | TGAGCGGCTTTACCTTTACCGAATATAGCATGCATTGGGTGCAGCAGGCGCCGGGCAAAGGCCTGGAATGG |
| | ATGGGCGGCATTAACCCGAACCAGGGCGGCACCAGCTATAACCAGAAATTTAAAGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 88) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H30 | TGAGCGGCTTTACCTTTACCGAATATAGCATGCATTGGGTGCAGCAGGCGCCGGGCAAAGGCCTGGAATGG |
| | ATGGGCGGCATTAACCCGCAGAACGCGGGCACCAGCTATAACCAGAAATTTAAAGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 98) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H31 | TGAGCGGCTTTACCTTTACCGAATATAGCATGCATTGGGTGCAGCAGGCGCCGGGCAAAGGCCTGGAATGG |
| | ATGGGCGGCATTAACCCGAACCAGGGCGGCACCAGCTATAACCAGAAATTTCAGGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 108) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H32 | TGAGCGGCTTTACCTTTACCGAATATAGCATGCATTGGGTGCAGCAGGCGCCGGGCAAAGGCCTGGAATGG |
| | ATGGGCGGCATTAACCCGCAGAACGCGGGCACCAGCTATAACCAGAAATTTCAGGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 118) |
| hACI-7069-633B12- | GAAGTGCAGCTGGTGCAGAGCGGCGCGGAAGTGAAAAAACCGGGCGCGACCGTGAAAATTAGCTGCAAAG |
| Ab1_H33 | TGAGCGGCTTTACCTTTACCGAATATAGCATGCATTGGGTGCAGCAGGCGCCGGGCAAAGGCCTGGAATGG |
| | ATGGGCGGCATTAACCCGAACAACGGCGGCACCAGCTATAACCAGAAATTTCAGGGCCGTGTGACCATTAC |
| | CGCGGATACCAGCACCGATACCGCGTATATGGAACTGAGCAGCCTGCGTAGCGAAGATACCGCGGTGTATT |
| | ATTGCGCGCGTGAAAGCTGGGGCCAGGGCACCACCGTGACCGTGAGCAGC |
| | (SEQ ID NO: 128) |

*DNA sequence optimized for mammalian cell expression.

TABLE 6
Amino acid sequence of the light chains (VL) and their CDRs

| Chain | Light chain | CDR L1 | CDR L2 | CDR L3 |
|---|---|---|---|---|
| ACI-7069-633B12- | DVVMTQTPLTLSVTIGQPASISCKSSQSLLHSDGKTYLNWLLQR | KSSQSLLHSDG | LVSKL | WQGTHF |
| Ab1_VL | PGQSPKRLIYLVSKLDSRIPDRFTGSGSGTDFTLKISRVEAEDLG | KTYLN (SEQ | DS | PHT |
| | VYYCWQGTHFPHTFGAGTKLELK | ID NO: 25) | (SEQ | (SEQ ID |
| | (SEQ ID NO: 24) | | ID NO: | NO: 27) |
| | | | 16) | |
| hACI-7069-633B12- | DVVMTQSPLSLPVTPGEPASISCKSSQSLLHSDGKTYLNWLLQK | KSSQSLLHSDG | LVSKL | WQGTHF |
| Ab1_L21 | PGQSPKRLIYLVSKLDSRVPDRFSGSGSGTDFTLKISRVEAEDVG | KTYLN | DS | PHT |
| | VYYCWQGTHFPHTFGQGTKLEIK | (SEQ ID NO: | (SEQ | (SEQ ID |
| | (SEQ ID NO: 34) | 25) | ID NO: | NO: 27) |
| | | | 16) | |
| hACI-7069-633B12- | DIVMTQSPLSLPVTPGEPASISCKSSQSLLHSDGKTYLNWLLQKP | KSSQSLLHSDG | LVSKL | WQGTHF |
| Ab1_L22 | GQSPKRLIYLVSKLDSRVPDRFSGSGSGTDFTLKISRVEAEDVG | KTYLN | DS | PHT |
| | VYYCWQGTHFPHTFGQGTKLEIK | (SEQ ID | (SEQ | (SEQ ID |
| | (SEQ ID NO: 44) | NO: 25) | ID NO: | NO: 27) |
| | | | 16) | |
| hACI-7069-633B12- | DVVMTQSPLSLPVTPGEPASISCKSSQSLLHSDGKTYLNWLLQK | KSSQSLLHSDG | LVSKL | WQGTHF |
| Ab1_L23 | PGQSPQRLIYLVSKLDSRVPDRFSGSGSGTDFTLKISRVEAEDVG | KTYLN | DS | PHT |
| | VYYCWQGTHFPHTFGQGTKLEIK | (SEQ ID | (SEQ | (SEQ ID |
| | (SEQ ID NO: 54) | NO: 25) | ID NO: | NO: 27) |
| | | | 16) | |
| hACI-7069-633B12- | DIVMTQSPLSLPVTPGEPASISCKSSQSLLHSDGKTYLNWLLQKP | KSSQSLLHSDG | LVSKL | WQGTHF |
| Ab1_L24 | GQSPQRLIYLVSKLDSRVPDRFSGSGSGTDFTLKISRVEAEDVG | KTYLN | DS | PHT |
| | VYYCWQGTHFPHTFGQGTKLEIK | (SEQ ID | (SEQ | (SEQ ID |
| | (SEQ ID NO: 64) | NO: 25) | ID NO: | NO: 27) |
| | | | 16) | |
| hACI-7069-633B12- | DIVMTQSPLSLPVTPGEPASISCKSSQSLLHSDGKTYLNWLLQKP | KSSQSLLHSDG | LVSKL | WQGTHF |
| Ab1_L25 | GQSPQLLIYLVSKLDSRVPDRFSGSGSGTDFTLKISRVEAEDVGV | KTYLN | DS | PHT |
| | YYCWQGTHFPHTFGQGTKLEIK | (SEQ ID | (SEQ | (SEQ ID |
| | (SEQ ID NO: 74) | NO: 25) | ID NO: | NO: 27) |
| | | | 16) | |
| hACI-7069-633B12- | DIVMTQSPLSLPVTPGEPASISCKSSQSLLHSDAKTYLNWLLQKP | KSSQSLLHSDA | LVSKL | FQGTHFP |
| Ab1_L26 | GQSPQRLIYLVSKLDSRVPDRFSGSGSGTDFTLKISRVEAEDVG | KTYLN | DS | HT |
| | VYYCFQGTHFPHTFGQGTKLEIK | (SEQ ID | (SEQ | (SEQ ID |
| | (SEQ ID NO: 84) | NO: 85) | ID NO: | NO: 87) |
| | | | 16) | |
| hACI-7069-633B12- | DIVMTQSPLSLPVTPGEPASISCKSSQSLLHSDGKTYLNWLLQKP | KSSQSLLHSDG | LVSKL | FQGTHFP |
| Ab1_L27 | GQSPQRLIYLVSKLDSRVPDRFSGSGSGTDFTLKISRVEAEDVG | KTYLN | DS | HT |
| | VYYCFQGTHFPHTFGQGTKLEIK | (SEQ ID | (SEQ | (SEQ ID |
| | (SEQ ID NO: 94) | NO: 25) | ID NO: | NO: 87) |
| | | | 16) | |

TABLE 7
DNA sequence of the humanized light chain variable domains (VL)

| Chain | Light chain |
|---|---|
| *ACI-7069-633B12- | GACGTGGTGATGACCCAGACCCCTCTGACACTGAGCGTGACCATCGGACAGCCCGCCTCCATCAGC |
| Ab1_VL | TGCAAAAGCTCCCAGTCTCTGCTGCACAGCGACGGCAAGACCTATCTGAACTGGCTGCTGCAGAGA |
| | CCCGGCCAAAGCCCCAAGAGGCTGATCTATCTGGTGAGCAAGCTGGACTCTAGAATCCCCGACAGA |
| | TTTACCGGCAGCGGCAGCGGCACAGACTTTACACTGAAGATCTCTAGAGTGGAGGCCGAAGATCTC |
| | GGCGTGTACTACTGCTGGCAAGGCACCCACTTCCCTCACACCTTCGGCGCCGGCACCAAGCTGGAG |
| | CTGAAG |
| | (SEQ ID NO: 29) |
| hACI-7069-633B12- | GATGTGGTGATGACCCAGAGCCCGCTGAGCCTGCCGGTGACCCCGGGCGAACCGGCGAGCATTAGG |
| Ab1_L21 | TGCAAAAGCAGCCAGAGCCTGCTGCATAGCGATGGCAAAACCTATCTGAACTGGCTGCTGCAGAAA |
| | CCGGGCCAGAGCCCGAAACGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTGGCAGGGCACCCATTTTCCGCATACCTTTGGCCAGGGCACCAAACTGGAA |
| | ATTAAA |
| | (SEQ ID N: 39) |
| hACI-7069-633B12- | GATATTGTGATGACCCAGAGCCCGCTGAGCCTGCCGGTGACCCCGGGCGAACCGGCGAGCATTAGC |
| Ab1_L22 | TGCAAAAGCAGCCAGAGCCTGCTGCATAGCGATGGCAAAACCTATCTGAACTGGCTGCTGCAGAAA |
| | CCGGGCCAGAGCCCGAAACGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTGGCAGGGCACCCATTTTCCGCATACCTTTGGCCAGGGCACCAAACTGGAA |
| | ATTAAA |
| | (SEQ ID NO: 49) |
| hACI-7069-633B12- | GATGTGGTGATGACCCAGAGCCCGCTGAGCCTGCCGGTGACCCCGGGCGAACCGGCGAGCATTAGC |
| Ab1_L23 | TGCAAAAGCAGCCAGAGCCTGCTGCATAGCGATGGCAAAACCTATCTGAACTGGCTGCTGCAGAAA |
| | CCGGGCCAGAGCCCGCAGCGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTGGCAGGGCACCCATTTTCCGCATACCTTTGGCCAGGGCACCAAACTGGAA |
| | ATTAAA |
| | (SEQ ID NO: 59) |
| hACI-7069-633B12- | GATATTGTGATGACCCAGAGCCCGCTGAGCCTGCCGGTGACCCCGGGCGAACCGGCGAGCATTAGC |
| Ab1_L24 | TGCAAAAGCAGCCAGAGCCTGCTGCATAGCGATGGCAAAACCTATCTGAACTGGCTGCTGCAGAAA |
| | CCGGGCCAGAGCCCGCAGCGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTGGCAGGGCACCCATTTTCCGCATACCTTTGGCCAGGGCACCAAACTGGAA |
| | ATTAAA |
| | (SEQ ID NO: 69) |
| hACI-7069-633B12- | GATATTGTGATGACCCAGAGCCCGCTGAGCCTGCCGGTGACCCCGGGCGAACCGGCGAGCATTAGG |
| Ab1_L25 | TGCAAAAGCAGCCAGAGCCTGCTGCATAGCGATGGCAAAACCTATCTGAACTGGCTGCTGCAGAAA |
| | CCGGGCCAGAGCCCGCAGCTGCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTGGCAGGGCACCCATTTTCCGCATACCTTTGGCCAGGGCACCAAACTGGAA |
| | ATTAAA |
| | (SEQ ID NO: 79) |
| hACI-7069-633B12- | GATATTGTGATGACCCAGAGCCCGCTGAGCCTGCCGGTGACCCCGGGCGAACCGGCGAGCATTAGC |
| Ab1_L26 | TGCAAAAGCAGCCAGAGCCTGCTGCATAGCGATGCGAAAACCTATCTGAACTGGCTGCTGCAGAAA |
| | CCGGGCCAGAGCCCGCAGCGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTTTCAGGGCACCCATTTTCCGCATACCTTTGGCCAGGGCACCAAACTGGAAA |
| | TTAAA |
| | (SEQ ID NO: 89) |
| hACI-7069-633B12- | GATATTGTGATGACCCAGAGCCCGCTGAGCCTGCCGGTGACCCCGGGCGAACCGGCGAGCATTAGC |
| Ab1_L27 | TGCAAAAGCAGCCAGAGCCTGCTGCATAGCGATGGCAAAACCTATCTGAACTGGCTGCTGCAGAAA |
| | CCGGGCCAGAGCCCGCAGCGTCTGATTTATCTGGTGAGCAAACTGGATAGCCGTGTGCCGGATCGT |
| | TTTAGCGGCAGCGGCAGCGGCACCGATTTTACCCTGAAAATTAGCCGTGTGGAAGCGGAAGATGTG |
| | GGCGTGTATTATTGCTTTCAGGGCACCCATTTTCCGCATACCTTTGGCCAGGGCACCAAACTGGAAA |
| | TTAAA |
| | (SEQ ID NO: 99) |

*DNA sequence optimized for mammalian cell expression.

### Example 2. Production of Humanized Antibody Variants

DNA coding sequence for both heavy and light chain variable domains were synthesized and cloned using standard molecular biology techniques into plasmid allowing the expression in mammalian cells. Heavy chain variable domains were fused to the human IgG1 constant domain and light chain variable domains were cloned into plasmid containing the constant Kappa light chain domain. The chimeric antibody and the humanized variants were transiently expressed in ExpiCHO-S(ThermoFischer scientific, A29127) cells by cotransfecting heavy and light chain plasmid using the ExpiCHO™ Expression System Kit (ThermoFischer scientific, A29133). Post transfection, cells were maintained at 37° C. under 150 rpm agitation and 8% C02 level. Six days after transfection, supernatants were harvested and purified by protein A (Cytiva, 17127903). Antibodies were captured by incubation with protein A at room temperature for 2 hours and under agitation on rollers. The mixture was poured into a gravity flow chromatography column (BioRad, 7321010), the resin was washed with 10CV of 2×PBS and eluted with 0.1M Glycine pH3.2. The elution was then neutralized by adding 0.1 M Tris, pH 7.4. The samples were then dialyzed in PBS buffer.

### Example 3. Characterization of ACI-7069-633B12-Ab1 Humanized Variants by Surface Plasmon Resonance (SPR)

Measurements were performed on a Biacore 8K instrument (GE Healthcare Life Sciences) by immobilizing soluble TDP-43 on a CM5 Series S sensor chip (GE Healthcare, BR-1005-30).

### KD Determination for Soluble TDP-43 by SPR

The instrument was primed with running buffer PBS-P+ and flow cells (Fc) 1 and 2 of channels 1-8 were activated with a fresh solution of EDC/NHS (Amine Coupling Kit, 1:1 ratio of both reagents, GE Healthcare Life Sciences, BR-1006-33) at 10 μL/min for 420 sec. Soluble TDP-43 (Selvita) was diluted in sodium acetate pH 4.5 to a final concentration of 5 μg/mL and injected for 900 sec with a flow rate of 5 μL/min on Fc 2. All flow cells were quenched with 1 M ethanol amine (GE Healthcare Life Sciences, BR-1006-33), at 10 μL/min for 420 sec. Immobilization levels after ethanolamine quenching were 680 RU on all eight channels. Prior to analysis, two Startup cycles were run. Increasing mAbs concentrations were injected in single-cycle kinetics ranging from 1.2 to 100 nM, prepared from a 3-fold serial dilution in running buffer, with a contact time of 300 sec and a dissociation time of 3600 sec at a flow rate of 30 μL/min. Each cycle was followed by one regeneration using 10 mM Glycine-HCl pH 1.7 with a contact time of 30 sec at 30 μL/min, followed by a stabilization period of 300 sec. Results obtained from single-cycle kinetics were double-referenced using the blank Fc 1 and buffer cycles and evaluated using Biacore 8K evaluation software using the 1:1 kinetic fit model with RI and global Rmax. The following kinetic parameters were obtained: association rate constant (ka), dissociation rate constant (kd), affinity constant (KD) and saturation response (Rmax).

### KD Determination for TP-51 Peptide by SPR

The instrument was primed with running buffer PBS-P+. Fc 1-2 of channels 1-8 (8K) were activated with a fresh solution of EDC/NHS (Amine Coupling Kit, 1:1 ratio of both reagents, GE Healthcare Life Sciences, BR-1006-33) at 10 μL/min for 420 sec and goat anti-human antibody (GE Healthcare Life Sciences, 29234600) was immobilized at 25 μg/mL in 10 mM sodium acetate pH 5 for 420 sec. Next, all Fc were quenched with 1 M ethanol amine (GE Healthcare Life Sciences, BR-1006-33), for 420 sec. Any non-covalently bound antibodies were removed by two regenerations with 10 mM Glycine-HCl pH 1.7 for 30 sec. Immobilization levels were evaluated following ethanolamine quenching (10000 RU for all channels).

Each cycle started with non-covalent capture of humanized variants which were diluted in running buffer to a final concentration of 2 μg/mL and injected for 60 sec with a flow rate of 10 μL/min. TDP-43 mAbs were captured on channels 1-8, leaving Fc 1 as a blank Fc. Capture levels were evaluated following a 120 sec stabilization period after each mAb injection and ranged from 300-500 RU.

TP-51 consists of amino acids 352-414 of TDP-43 (SEQ ID NO: 1). Injections of TP-51 peptide (Pepscan) were performed in single-cycle kinetics with increasing concentrations ranging from 1.2-100 nM prepared from serial 3-fold dilutions. Injections were performed with contact times of 300 sec/injection at a flow rate of 25 μL/min. A dissociation phase of 3600 sec followed the final injection. Sensor surface was regenerated by one injection of 10 mM Glycine-HCl pH 1.7 at a flow rate of 10 μL/min for 120 sec, followed by a stabilization period of 300 sec. Results obtained from single-cycle kinetics were double-referenced using the blank Fc 1 and buffer cycles and evaluated by Biacore 8K evaluation software with 1:1 kinetic fit model with RI and global Rmax. The following kinetic parameters were obtained: association rate constant (ka), dissociation rate constant (kd), affinity constant (KD) and saturation response (Rmax).

All parameters (except Rmax) are reported in Table 8 as mean±SD from 2-6 independent experiments.

TABLE 8
ka, kd, KD value of ACI-7069-633B12-Ab1 humanized
variants on soluble TDP-43 and TP-51 peptide

| | TP-51 | | | TDP-43 | | |
|---|---|---|---|---|---|---|
| | ka | kd | KD | ka | kd | KD |
| mAb | (1/Ms) | (1/s) | (nM) | (1/Ms) | (1/s) | (pM) |
| ACI-7069-633B12-Ab1 | 6.7E+04 | 9.8E−05 | 1.5 | 5.5E+04 | 1.7E−05 | 328 |
| hACI-7069-633B12- | 4.2E+04 | 1.4E−04 | 3.3 | 2.4E+04 | 2.2E−05 | 925 |
| Ab1_H27L23 | | | | | | |
| hACI-7069-633B12- | 5.8E+04 | 2.0E−04 | 3.5 | 2.9E+04 | 3.4E−05 | 1156 |
| Ab1_H28L22 | | | | | | |
| hACI-7069-633B12- | 6.5E+04 | 1.3E−04 | 1.9 | 3.2E+04 | 2.6E−05 | 806 |
| Ab1_H28L23 | | | | | | |
| hACI-7069-633B12- | 6.6E+04 | 1.3E−04 | 2.0 | 3.7E+04 | 1.7E−05 | 531 |
| Ab1_H28L24 | | | | | | |
| hACI-7069-633B12- | 6.4E+04 | 1.2E−04 | 2.0 | 2.4E+04 | 1.5E−05 | 716 |
| Ab1_H32L24 | | | | | | |
| hACI-7069-633B12- | 1.2E+05 | 1.6E−04 | 1.4 | 3.3E+04 | 1.7E−05 | 563 |
| Ab1_H32L26 | | | | | | |
| hACI-7069-633B12- | 7.0E+04 | 1.5E−04 | 2.2 | 3.0E+04 | 1.3E−05 | 471 |
| Ab1_H33L24 | | | | | | |
| hACI-7069-633B12- | 1.6E+05 | 2.9E−04 | 1.6 | 4.3E+04 | 9.0E−06 | 220 |
| Ab1_H33L26 | | | | | | |
| hACI-7069-633B12- | 1.2E+05 | 1.2E−04 | 1.0 | 3.6E+04 | 5.2E−06 | 143 |
| Ab1_H32L27 | | | | | | |
| hACI-7069-633B12- | 1.5E+05 | 1.2E−04 | 0.8 | 5.2E+04 | 6.5E−06 | 126 |
| Ab1_H33L27 | | | | | | |

Overall, all humanized variants retained good affinity to TDP-43 and TP-51 peptide (Table 8) as observed for the murine antibody ACI-7069-633B 12-Ab1. The variants with the best affinity from the variants tested in this set of experiments were hACJ-7069-633B12-Ab1_H28L24, hACI-7069-633B 12-Ab1_H33L24, hACI-7069-633B 12-Ab1_H32L26, hACI-7069-633B 12-Ab1_H33L26, hACI-7069-633B12-Ab1_H32L27 and hACI-7069-633B12-Ab1_H33L27.

Humanized variants presented similar binding affinity to TDP-43 (ranging from 1156 to 126 pM, Table 8) compared to the murine parental antibody ACI-7069-633B 12-Ab1 (328 pM, Table 8), with hACI-7069-633B12-Ab1_H33L27 having the best affinity to TDP-43 (lowest KD). Of note, the change in pI and net charge of the humanized variant hACI-7069-633B 12-Ab11_H33L27 did not impact its thermostability, measured by Differential Scanning Fluorimetry (DSF), that remained above 70° C. as expected for standard human IgG1, nor its affinity for FcRn.

### Example 4. ACI-7069-633B12-Ab1 Humanized Variants Inhibit TDP-43 Aggregation In Vitro

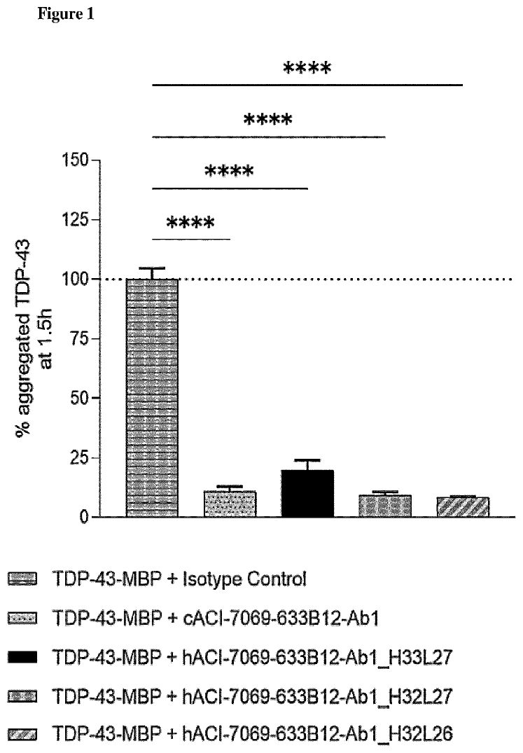

hACI-7069-633B12-Ab1_H33L27, hACI-7069-633B12-Ab1_H32L27 and hACI-7069-633B12-Ab1_H32L26 were characterised in an in vitro functional assay relying on the aggregation property of TDP-43 (Wang et al. EMBO 2018). For this, TDP-43 was fused with maltose binding protein at the C-terminus (TDP-43-MBP), produced recombinantly and aggregation was induced by the removal of the MBP protein using Tobacco Etch Virus (TEV) protease. Aggregation was then monitored over time by measuring absorbance at 600 nm. The increase in absorbance at 600 nm is reported to correlate with the aggregate amounts. At 1.5 hours, hACI-7069-633B12-Ab1_H33L27, hACI-7069-633B12-Ab1_H32L27 and hACI-7069-633B12-Ab1_H32L26 maintained functional efficacy in inhibition of TDP-43 aggregation, similar to the human IgG chimera cACI-7069-633B12-Ab1 antibody (chimera of the VH and VL of ACI-7069-633B12-Ab1 murine antibody and human IgG1 constant region). In contrast, the addition of an isotype Control mAb did not inhibit TDP-43 aggregation (FIG. 1).

### Example 5. Pharmacokinetic Evaluation of Two ACI-7069-633B12-Ab1 Humanized Variants in Tg32 Mice

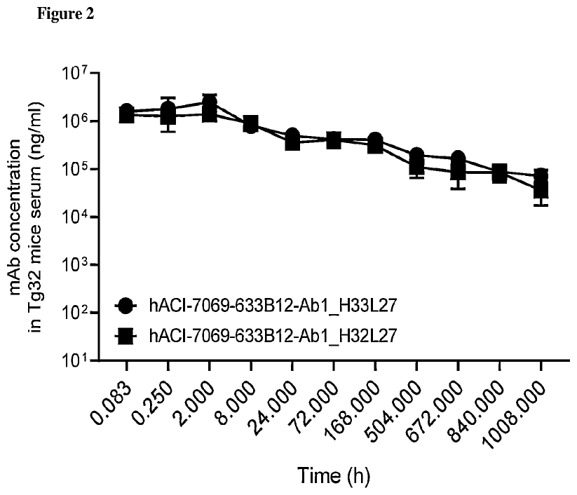

Transgenic Tg32 mice overexpress the human neonatal Fc receptor and present a strong interest for PK evaluation and projected human PK modelling (Avery et al., Mabs 2016). A single IV bolus (40 mg/kg) of either hACI-7069-633B12-Ab1_H33L27 or hACI-7069-633B12-Ab1_H32L27 was given to 21 male Tg32 mice per compound. For 6 weeks, blood sampling was performed (as shown on the y-axis in FIG. 2) and serum (3 mice per time point) was analysed. Overall, both antibodies presented good and similar PK parameters which make them desirable candidates for applications in which the half-life of the antibody in vivo is important, for example for therapeutic use in humans (FIG. 2, Table 9).

TABLE 9
Tg32 mice PK parameters

| | | hACI-7069-633B12- | hACI-7069-633B12- |
|---|---|---|---|
| Parameter | Unit | Ab1_H33L27 | Ab1_H32L27 |
| CL | ml/h/kg | 0.18 | 0.25 |
| Vc | ml/kg | 20.8 | 34.3 |
| Q1 | ml/h/kg | 2.8 | 2.6 |
| Vp1 | ml/kg | 79.0 | 69.5 |
| T_{1/2 β} | days | 16.4 | 12.5 |

CL: systemic clearance;
Vc: central volume of distribution;
Q1: intercompartmental clearance;
Vp1: peripheral volume of distribution;
T_{1/2 β}: terminal half-life.

### Example 6. Pharmacokinetic Evaluation of Two ACI-7069-633B12-Ab1 Humanized Variants in Cynomolgus Monkeys

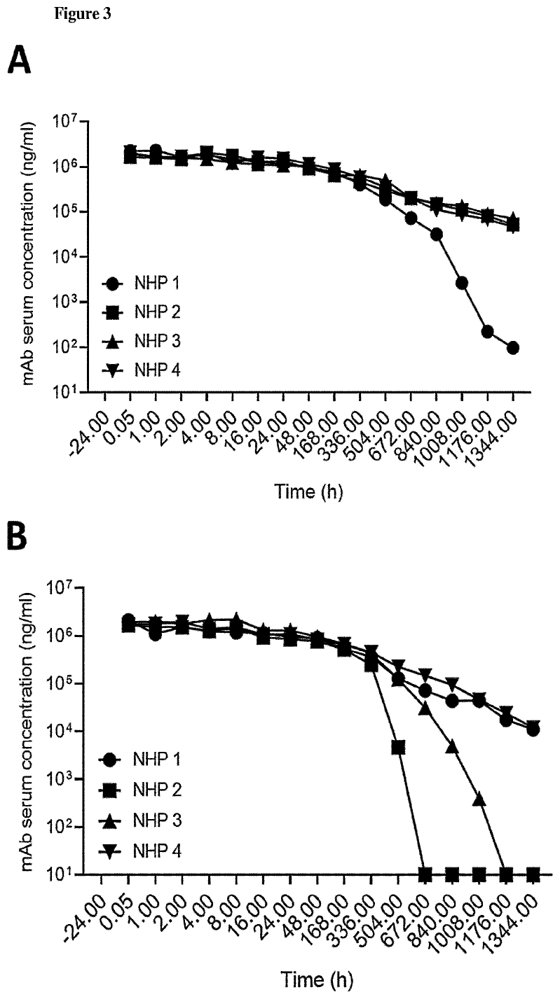

To assess the pharmacokinetics of hACI-7069-633B12-Ab1_H33L27 and hACI-7069-633B12-Ab1_H32L27 in non-human primates, a single IV bolus (40 mg/kg) was given to 4 males cynomolgus monkeys per compound. Over a period of 56 days (1344h), blood sampling was performed (as shown on the y-axis in FIG. 3) and serum (4 cynomolgus monkey samples per time point) was analysed. 3 of 8 animals were detected as having anti-drug antibodies (ADA, confirmed by ELISA), a percentage in line with that described previously in non-human primates (NHPs) for human IgGs administration (Valente et al., Mabs 2020). After exclusion of animals with ADA, PK parameters were calculated. Both antibodies displayed favorable PK parameters (FIG. 3, Table 10) making them suitable for further clinical development as therapeutic candidates.

TABLE 10
NHP PK parameters

| | | hACI-7069-633B12- | hACI-7069-633B12- |
|---|---|---|---|
| Parameter | Unit | Ab1_H33L27 | Ab1_H32L27 |
| CL | ml/h/kg | 0.08 | 0.12 |
| Vc | ml/kg | 23.3 | 22.5 |
| Q1 | ml/h/kg | 0.4 | 1.4 |
| Vp1 | ml/kg | 12.3 | 15.1 |
| T_{1/2 β} | days | 12.7 | 8.7 |

CL: systemic clearance;
Vc: central volume of distribution;
Q1: intercompartmental clearance;
Vp1: peripheral volume of distribution;
T_{1/2 β}: terminal half-life.

To assess preliminary tolerability, toxicity and toxicokinetic data in non-human primates, hACI-7069-633B12-Ab1_H33L27 was administered intravenously once weekly for four weeks to cynomolgus monkeys at doses of 40, 120 and 360 mg/kg. Animals were sacrificed one week after the last administration. Serum samples were taken at various timepoints and analyzed for hACI-7069-633B12-Ab1_H33L27 concentration. A dose proportional increase in hACI-7069-633B12-Ab1_H33L27 exposure in serum was observed with no sign of immunogenicity during the study duration (FIG. 9). Moreover, no adverse effects were observed on body weight, clinical observations, clinical pathology, urinalysis, microscopic examinations or organ weights of the cynomolgus monkeys, confirming the suitability of ACI-7069-633B12-Ab1 humanized variants for further clinical development.

### Example 7. Comparison of Pharmacokinetic Profile of ACI-7069-633B12-Ab1_H33L27 to Parental Antibodies in Tg32 Mice

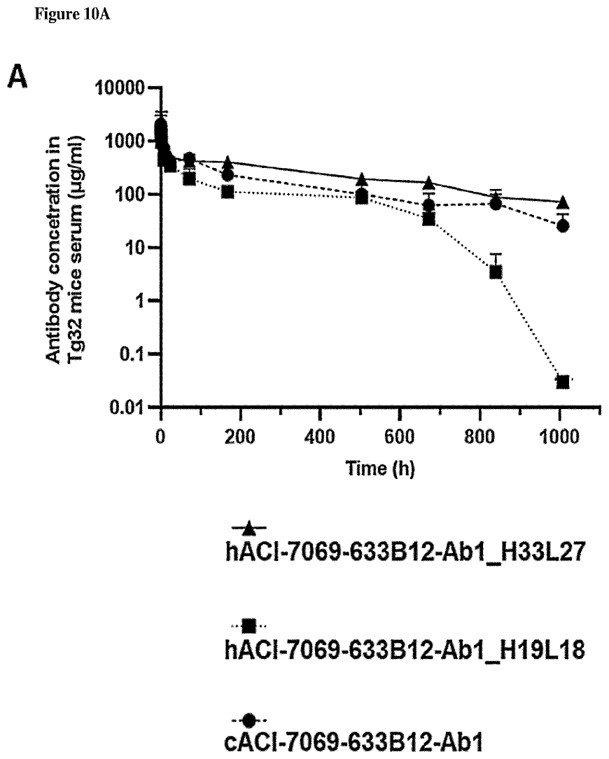

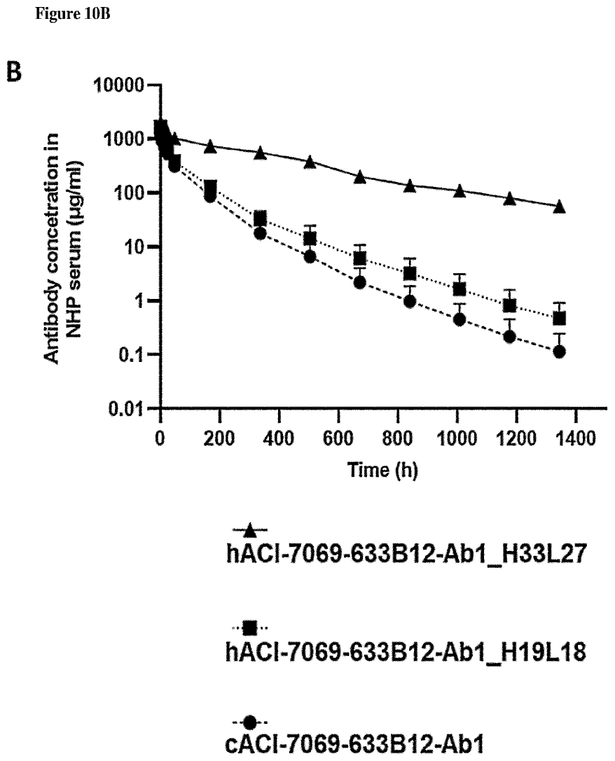

To further assess the pharmacokinetics of ACI-7069-633B12 humanized variants, a single IV bolus (40 mg/kg) of either hACI-7069-633B12-Ab1_H33L27, hACI-7069-633B12-Ab1_H19L18 (as described in WO2022/034228) or cACI-7069-633B12-Ab1 was given to 21 male Tg32 mice per compound. Blood sampling and serum analysis was performed as described in Example 5. Data are depicted as the mean±standard deviation for 3-4 animals/group (FIG. 10A). Humanized variant ACI-7069-633B12-Ab1_H33L27 presented a superior PK profile compared to parental hACI-7069-633B12-Ab1_H19L18 and cACI-7069-633B12-Ab1 antibodies (FIG. 10A), reasserting its suitability for further clinical development.

### Example 8. Comparison of Pharmacokinetic Profile of ACI-7069-633B12-Ab1_H33L27 to Parental Antibodies in Cynomolgus Monkeys

The pharmacokinetics of ACI-7069-633B12 humanized variants was further assessed in NHP with a single IV bolus administration (40 mg/kg) of either hACI-7069-633B12-Ab1_H33L27, hACI-7069-633B12-Ab1_H19L18 (as described in WO2022/034228) or cACI-7069-633B12-Ab1 (4 males cynomolgus monkeys per antibody). Blood sampling and serum analysis was performed as described in Example 6. Data are depicted as the mean±standard deviation for 3-4 animals/group (FIG. 10B). As seen in Tg32 mice, humanized variant ACI-7069-633B12-Ab1_H33L27 presented a superior PK profile compared to parental hACI-7069-633B12-Ab1_H19L18 and cACI-7069-633B12-Ab1 antibodies (FIG. 10B). This result corroborated the suitable PK profile of hACI-7069-633B12-Ab1_H33L27 for further clinical development.

### Example 9. Human PK Prediction from Tg32 Mice and Cynomolgus Monkeys

Tg32 mice and cynomolgus monkeys were used as in vivo tools for prediction of mAb human clearance. PK parameters estimated from Tg32 and cynomolgus monkey were scaled to 70 kg human by allometric scaling using fixed exponent as reported in the literature (Table 11) (Betts et al., 2018). The predicted human clearance in Tg32 mice and cynomolgus monkeys was 0.195 mL/h/kg and 0.256 mL/h/kg for hACI-7069-633B12-Ab1_H19L18 (described in WO2022/034228) versus 0.082 mL/h/kg and 0.043 mL/h/kg for hACI-7069-633B12-Ab1_H33L27, respectively (Table 11). It resulted in predicted human half-life of 27-30 days for hACI-7069-633B12-Ab1_H33L27 versus 17-22 days for hACI-7069-633B12-Ab1_H19L18, respectively, and confirmed favorable predicted human PK profile of hACI-7069-633B12-Ab1_H33L27.

TABLE 11
Predicted human PK parameters

| | | | hACI-7069- | hACI-7069- |
|---|---|---|---|---|
| Prediction | | | 633B12- | 633B12- |
| from | Parameter | Unit | Ab1_H33L27 | Ab1_H19L18 |
| Tg32 mice | CL | ml/h/kg | 0.082 | 0.195 |
| | Vc | ml/kg | 16.4 | 23.2 |
| | Q1 | ml/h/kg | 0.2 | 0.18 |
| | Vp1 | ml/kg | 45.1 | 66.2 |
| | T_{1/2 β} | days | 27 | 22 |
| Cynomolgus | CL | ml/h/kg | 0.043 | 0.256 |
| monkeys | Vc | ml/kg | 26.7 | 29.6 |
| | Q1 | ml/h/kg | 0.1 | 0.104 |
| | Vp1 | ml/kg | 15.6 | 39.8 |
| | T_{1/2 β} | days | 30.2 | 16.7 |

CL: systemic clearance;
Vc: central volume of distribution;
Q1: intercompartmental clearance;
Vp1: peripheral volume of distribution;
T_{1/2 β}: terminal half-life.

### Example 10. NHP Serum Pharmacodynamics of Two ACI-7069-633B12-Ab1 Humanized Variants

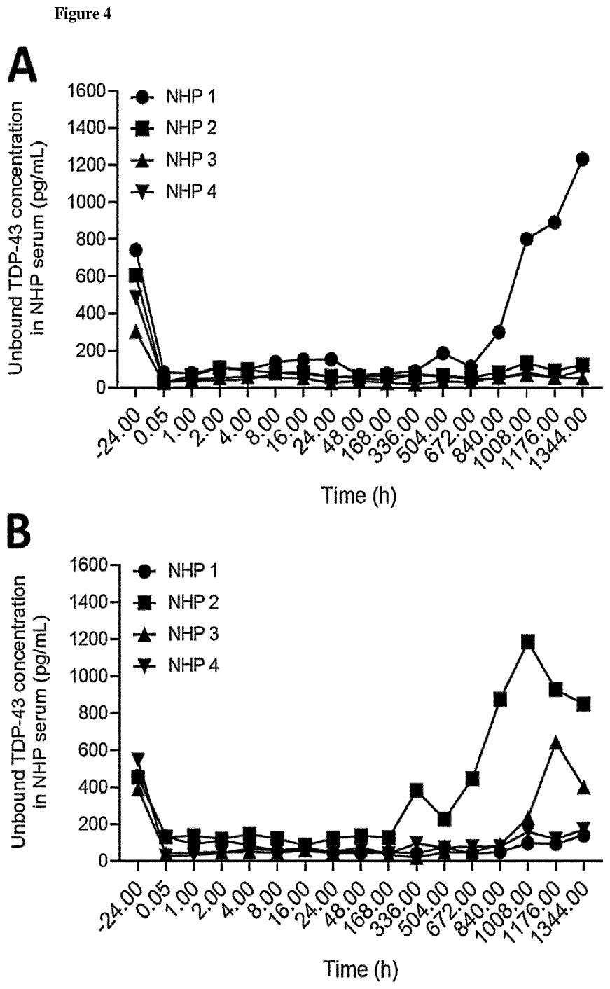

To assess serum pharmacodynamics, unbound TDP-43 levels were measured in NHPs dosed with the compounds using Single Molecule Array (SIMOA®) technology. An average of 500 μg/ml of TDP-43 was measured at the pre-dosed time points (FIG. 4A-B) as baseline TDP-43 levels. A fast drop in free/unbound TDP-43 (more than 80%) was measured 3 minutes after the IV injection of either hACI-7069-633B12-Ab1_H33L27 or hACI-7069-633B12-Ab1_H32L27 (0.05h), indicating a complete and fast target saturation by these two mAbs, with the native TDP-43 protein present in the serum of these NHPs. TDP-43 stayed bound to the antibodies until the end of the study, except in the animals presenting ADA (as described in Example 6), demonstrating target engagement of the administered antibodies (FIG. 4A-B).

### Example 11. Neutralization of Seeding-Competent TDP-43 in Sporadic ALS Cerebrospinal Fluid by One ACI-7069-633B12-Ab1 Humanized Variant

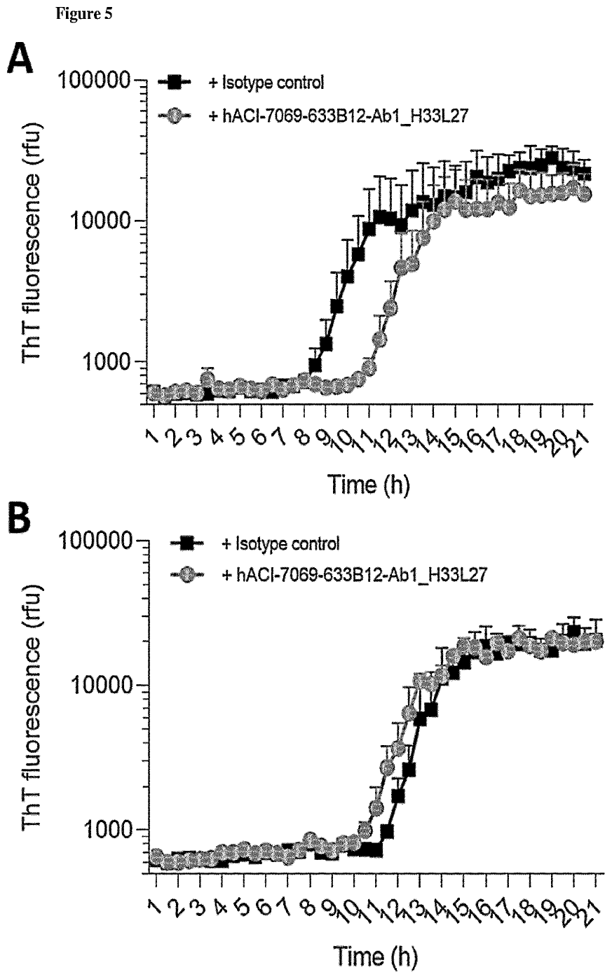

To confirm the potency and mode of action of ACI-7069-633B12-Ab1 humanized variants, a real-time quaking-induced conversion (RT-QuIC) assay was performed as previously described (Scialo, C. et al. Brain Commun, fcaa142-(2020)), using a synthetic TDP-43 peptide (TP-51) as substrate (used at 10 μM). The observed accelerated aggregation of the substrate in the presence of cerebrospinal fluid (CSF) from sporadic ALS donors (FIG. 5A) compared to healthy controls (FIG. 5B) confirmed the presence of seeding-competent TDP-43 species in ALS CSF. Each CSF, used as seeds in this assay, was then pre-incubated with either hACI-7069-633B12-Ab1_H33L27 or isotype control (used at 0.006 PM). A significant delay in the aggregation of the TDP-43 peptide (TP-51) substrate was observed with the ACI-7069-633B12-Ab1_H33L27 humanized variant in CSF from ALS donors (FIG. 5A-B), indicating that the antibody can bind and neutralize seeding-competent TDP-43 in ALS patients' CSF.

### Example 12. ACI-7069-633B12-Ab1 Humanized Variants Potentiate TDP-43 Uptake by THP-1 Cells

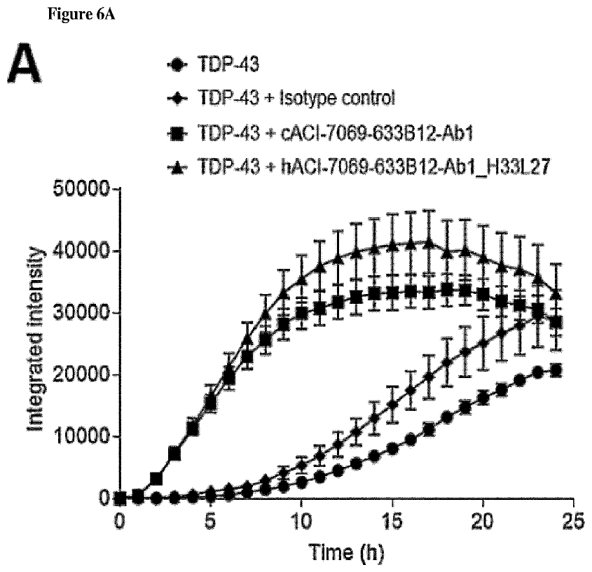

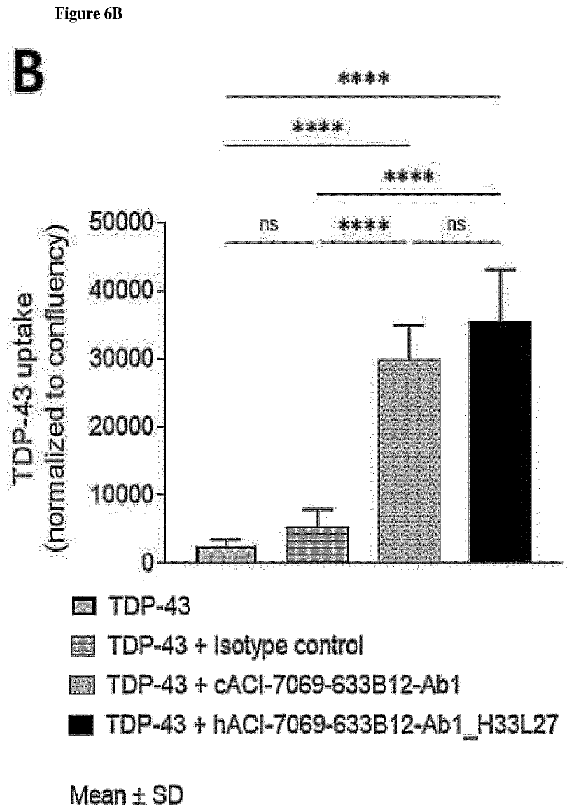

To evaluate and confirm the direct involvement of microglia in clearing TDP-43 aggregates by Fc-dependent mechanisms, an in vitro assay using THP-1 cells (a human leukemia monocytic cell line) was set up as previously described (Lindner et al., 2020). For this, recombinant TDP-43 aggregates were labeled with pHrodo™ dye (ThermoFisher Scientific, P36013) according to the manufacturer's instructions. The pHrodo™ dye becomes fluorescent upon internalization in an acidic cellular compartment allowing the monitoring of internalization of TDP-43 aggregates by THP-1 cells in real time. pHrodo™ fluorescence intensity was then automatically quantified every hour over a 24 h period (FIG. 6A). Data represent mean±SEM with 4 to 6 replicates per condition. The difference in fluorescence intensity normalized to cell confluency for the 10 h time point was compared between conditions with a one-way ANOVA followed by a Tukey post hoc test for multiple comparisons (FIG. 6B). Limited internalization of TDP-43 aggregates was observed upon incubation with 30 nM of TDP-43 aggregates alone or in the presence of 30 nM of an isotype control antibody. However, in the presence of 30 nM of either hACI-7069-633B12-Ab1_H33L27 or the chimeric antibody cACI-7069-633B12-Ab1, a significant increase in the uptake of pHrodo™-labeled TDP-43 aggregates was observed compared to the negative controls pHrodo™-labeled TDP-43 alone and in combination with isotype control antibody (FIG. 6A-B). These results confirmed that binding of ACI-7069-633B12 humanized variants to TDP-43 aggregates can potentiate their uptake by immune cells such as microglia, and therefore their clearance.

### Example 13. TDP-43 Immunodepletion from Human Brain Extract by ACI-7069-633B12-Ab1 Humanized Variants

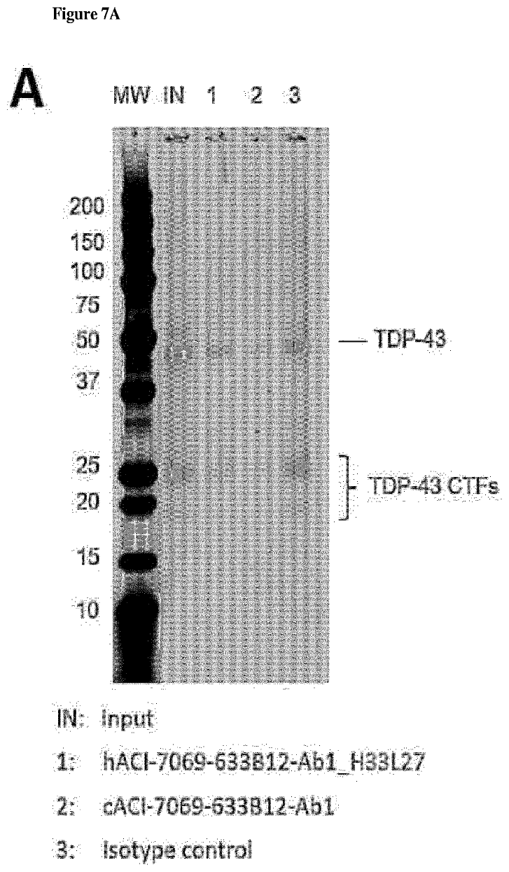

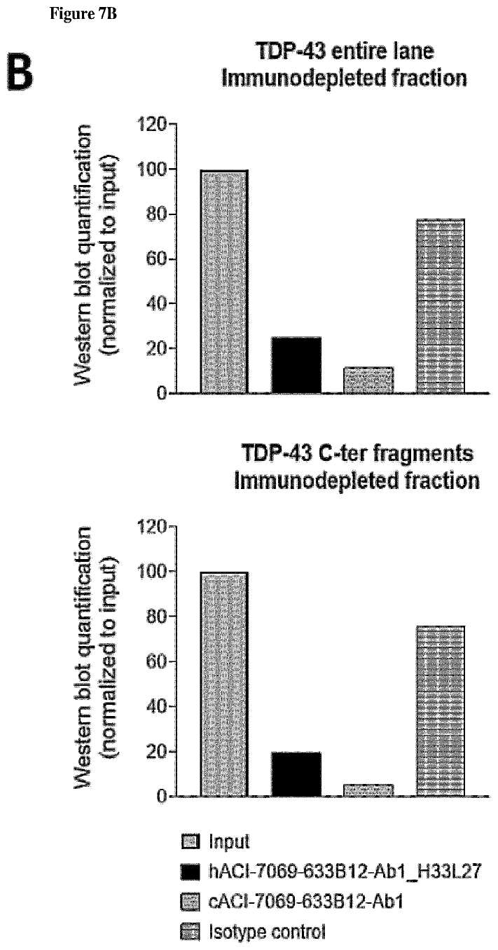

To evaluate the effect of ACI-7069-633B12-Ab1 humanized variants on templated TDP-43 aggregation in vitro, an immunodepletion experiment was performed with aggregated and phosphorylated TDP-43 enriched from an FTLD-TDP brain extract as previously described (WO2022/034228). The immunodepleted fractions demonstrated substantial TDP-43 depletion using either hACI-7069-633B12-Ab1_H33L27 or the chimeric antibody cACI-7069-633B12-Ab1 compared to the isotype control (FIG. 7A). For quantitative analyses, the signals obtained for total TDP-43 or C-terminal fragments of TDP-43 (i.e., lower than 25 kDa, CTF) were normalized to the corresponding signal in the input (FIG. 7B). These results confirmed the ability of ACI-7069-633B12-Ab1 humanized variants to bind aggregated and phosphorylated TDP-43 from the brain of FTLD-TDP patients, confirming its therapeutic potential.

### Example 14. ACI-7069-633B12-Ab1 Humanized Variants Bind Human TDP-43 in CSF with High Affinity

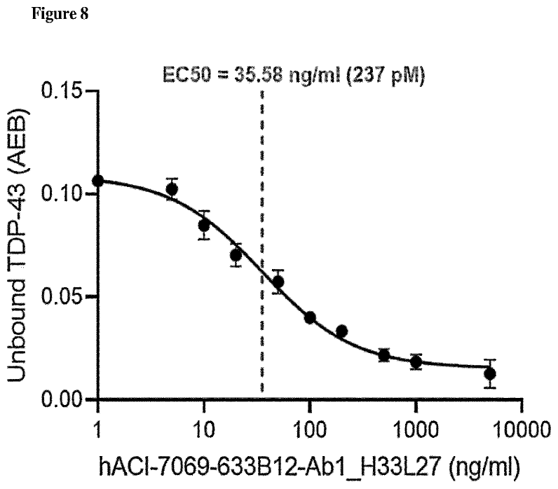

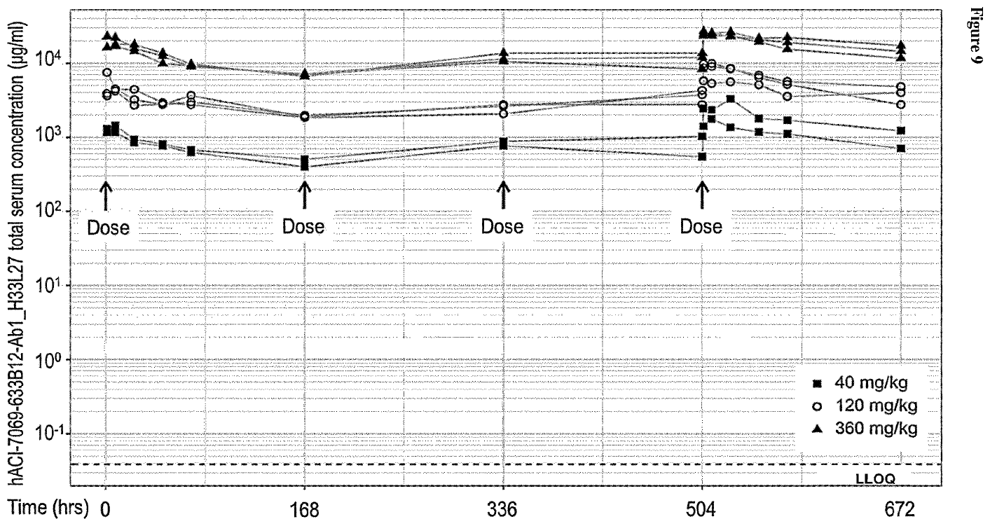

In order to characterize the binding affinity of ACI-7069-633B12-Ab1 humanized variants for TDP-43 in human CSF, a CSF sample from a healthy donor was pre-incubated with increasing concentrations of hACI-7069-633B12-Ab1_H33L27 and the amount of unbound TDP-43 was measured using a SIMOA-based target engagement assay as in Example 10. An EC50 of 35.58 ng/ml (237 pM) was obtained for hACI-7069-633B12-Ab1_H33L27 binding to human TDP-43 in CSF samples (FIG. 8). This result confirmed the ability of ACI-7069-633B12-Ab1 humanized variants to bind TDP-43 in CSF with high affinity.

### REFERENCES

Arai et al., TDP-43 is a component of ubiquitin-positive tau-negative inclusions in frontotemporal lobar degeneration and amyotrophic lateral sclerosis, Biochemical and Biophysical Research Communications 351 (2006) 602-611.
Avery et al., Utility of a human FcRn transgenic mouse model in drug discovery for early assessment and prediction of human pharmacokinetics of monoclonal antibodies, mAbs, 8:6, 1064-1078, DOI: 10.1080/19420862.2016.1193660
Betts et al., Linear pharmacokinetic parameters for monoclonal antibodies are similar within a species and across different pharmacological targets: A comparison between human, cynomolgus monkey and hFcRn Tg32 transgenic mouse using a population-modeling approach, MABS, 2018, Vol. 10
Buratti and Baralle, Nuclear factor TDP-43 can affect selected microRNA levels, FEBS Journal 277 (2010) 2268-2281.
Brettschneider J et al., Spreading of pathology in neurodegenerative diseases: a focus on human studies, Nature Rev. Neuroscience, 2015, 109.
Brettschneider et al., Stages of pTDP-43 pathology in amyotrophic lateral sclerosis, Ann Neurol. 2013 July; 74(1): 20-38.
Bumbaca Yadav et al., Evaluating the Use of Antibody Variable Region (Fv) Charge as a Risk Assessment Tool for Predicting Typical Cynomolgus Monkey Pharmacokinetics, The journal of biological chemistry, VOL. 290, NO. 50, pp. 29732-29741, 2015
Charlton, Methods in Molecular Biology, Val. 248 (B. K. C. Lo, ed., Humana Press, Totowa, NJ, 2003), pp. 245-254.
Chowdhury, Methods Mol. Biol. 207:179-196 (2008)
Clynes et al., Fc receptors are required in passive and active immunity to melanoma Proc. Nat'l Acad. sci. USA 95:652-656 (1998)
Cohen et al., An acetylation switch controls TDP-43 function and aggregation propensity, Nat Commun. 6: 5845, 2015.
Cragg, M. S. et al., Complement-mediated lysis by anti-CD20 mAb correlates with segregation into lipid rafts, Blood 101:1045-1052 (2003)
Cragg, M. S. and M. J. Glennie, Antibody specificity controls in vivo effector mechanisms of anti-CD20 reagents, Blood 103:2738-2743 (2004)).
Cunningham and Wells, High-resolution epitope mapping of hGH-receptor interactions by alanine-scanning mutagenesis, (1989) Science, 244: 1081-1085.
Dall' Acqua et al., Increasing the Affinity of a Human IgG1 for the Neonatal Fe Receptor: Biological Consequences, Journal of immunology, (2002) 169(9):5171-5180.
Duncan & Winter, The binding site for C1q on IgG, Nature 322:738-40 (1988)
Edelman, G. M. et al., The Covalent Structure of an Entire gammaG Immunoglobulin Molecule, Proc. Natl. Acad. USA, 63, 78-85 (1969)
Feiler et al., TDP-43 is intercellularly transmitted across axon Terminals, J. Cell Biol. Vol. 211 No. 4 897-911.
Feneberg et al., Towards a TDP-43-Based Biomarker for ALS and FTLD, Molecular Neurobiology, 2018; 55(10): 7789-7801.
Gazzano-Santoro et al., Engineered Antibodies with Increased Activity to Recruit Complement, J. Immunol. Methods 202:163 (1996)
Gerngross, Advances in the production of human therapeutic proteins in yeasts and filamentous fungi, Nat. Biotech. 22:1409-1414 (2004).
Gerhardt et al., Methods for General and Molecular Bacteriology, ASM Press (1994).
Golemis, Protein-Protein Interactions: A Molecular Cloning Manual, Cold Spring Harbor Laboratory Press (2002).
Graham et al., Characteristics of a human cell line transformed by DNA from human adenovirus type 5, J. Gen Viral. 36:59 (1977)
Guyer et al., Immunoglobulin binding by mouse intestinal epithelial cell receptors, J. Immunol. 117:587 (1976).
Harlow and Lane, Antibodies: A Laboratory Manual, Cold Spring Harbor Laboratory Press, (1988).
Harlow and Lane, Using Antibodies: A Laboratory Manual, Cold Spring Harbor Laboratory Press, (1999).
Hasegawa et al., Phosphorylated TDP-43 in frontotemporal lobar degeneration and amyotrophic lateral sclerosis, (2008) Annals of Neurology Vol 64 No 1, 60-70.
Hasegawa et al., Prion-like mechanisms and potential therapeutic targets in neurodegenerative disorders, Pharmacol Ther. 2017 April; 172:22-33.
Hellstrom, I. et al., Proc. Nat'l Acad. Sci. USA 83:7059-7063 (1986).
Hellstrom, I et al., Proc. Nat'l Acad. Sci. USA 82:1499-1502 (1985).
Hoogenboom et al., Methods in Molecular Biology 178:1-37 (O'Brien et al., ed., Human Press, Totowa, NJ, (2001).
Howard and Bethell. (2000) Basic Methods in Antibody Production and Characterization, Crc. Pr. Inc.
Idusogie et al., J. Immunol. 164: 4178-4184 (2000).
Igawa et al, Reduced elimination of IgG antibodies by engineering the variable region, PEDS. vol. 23 no. 5 pp. 385-392, 2010
Ishii et al., Formation and spreading of TDP-43 aggregates in cultured neuronal and glial cells demonstrated by time-lapse imaging, PLoS ONE 12(6): e0179375, 2017.
Jones et al., Replacing the complementarity-determining regions in a human antibody with those from a mouse, Nature 321 (1986), 522-525.
K A et al., TDP-43 is a key player in the clinical features associated with Alzheimer's disease, Acta Neuropathol. 2014; 127(6): 811-824.
K A et al., Staging TDP-43 pathology in Alzheimer's disease Acta Neuropathol. 2014; 127(3): 441-450.
Kam et al., Proc. Natl. Acad. Sci. USA 102: 11600-11605 (2005).
Kanda Y. et al., Bioteehnol. Bioeng., 94(4):680-688 (2006).
Kabat et al., Sequences of Proteins of Immunological Interest, 5th edit. NIH Publication no. 91-3242 U.S. Department of Health and Human Services (1991).
Kasai et al. “Combined use of CSF NfL and CSF TDP-43 improves diagnostic performance in ALS.” Annals of clinical and translational neurology vol. 6,12 (2019): 2489-2502. doi:10.1002/acn3.50943
Kirn et al., J. Immunol. 24:249 (1994).
Kohler, Nature 256 (1975), 495.
Khor, Proceedings of the American Society of Clinical Oncology Abstract (1997), 847.
Lagier-Tourenne et al., TDP-43 and FUS/TLS: emerging roles in RNA processing and neurodegeneration, Human Molecular Genetics, 2010, Vol. 19, Review Issue 1 R46-R64.
Lagier-Tourenne and Cleveland, Rethinking ALS: the FUS about TDP-43, Cell 136, 2009, 1001-1004.
Le Ber, Genetics of frontotemporal lobar degeneration: an up-date and diagnosis algorithm, Revue Neurologique 169 (2013) 811-819.
Lefkovits, Immunology Methods Manual: The Comprehensive Sourcebook of Techniques; Academic Press (1997).
Lindner B, Burkard T, Schuler M. Phagocytosis assays with different pH-sensitive fluorescent particles and various readouts. Biotechniques. 2020 February; 68(5):245-50.
LoBuglio, Proceedings of the American Society of Clinical Oncology Abstract (1997), 1562. Li et al., Optimization of humanized IgGs in glycoengineered Pichia pastoris, Nat. Biotech. 24:210-215 (2006).
Mackenzie and Neumann, Molecular neuropathology of frontotemporal dementia: insights into disease mechanisms from postmortem studies, J. Neurochem. (2016) 138 (Suppl. 1), 54-70.
Mather, Establishment and characterization of two distinct mouse testicular epithelial cell lines, Biol. Reprod. 23:243-251 (1980)
Mather et al., Culture of testicular cells in hormone-supplemented serum-free medium, Annals N. Y Aead. Sei. 383:44-68 (1982)
McAleese et al., TDP-43 pathology in Alzheimer's disease, dementia with Lewy bodies and ageing, Brain Pathol. 2017 July; 27(4): 472-479.
Morris, Epitope Mapping Protocols, Methods in Molecular Biology vol. 66(1996) (Humana Press, Totowa, NJ).
Nonaka et al., Prion-like Properties of Pathological TDP-43 Aggregates from Diseased Brains, Cell Reports 4 (2013), 124-134.
Neumann et al., Ubiquitinated TDP-43 in frontotemporal lobar degeneration and amyotrophic lateral sclerosis, Science 314, (2006), 130-133.
Neumann et al., Phosphorylation of S409/410 of TDP-43 is a consistent feature in all sporadic and familial forms of TDP-43 proteinopathies, Acta Neuropathol. (2009) 117: 137-149.
Petkova, S. B. et al., Int'l. Immunol. 18(12):1759-1769 (2006).
Pluckthun, The Pharmacology of Monoclonal Antibodies, Rosenburg and Moore eds. Springer-Verlag, N.Y. (1994), 269-315.
Porta S. et al., Patient-derived frontotemporal lobar degeneration brain extracts induce formation and spreading of TDP-43 pathology in vivo Nat. Comm., 2018
Okazaki et al., Fucose depletion from human IgG1 oligosaccharide enhances binding enthalpy and association rate between IgG1 and FcgammaRIIIa. J. Mol. Biol. 336:1239-1249 (2004).
Presta L G., Antibody engineering, Curr Op Struct Biol 2 (1992), 593-596.
Ravetch and Kinet, Annu. Rev. Immunol. 9:457-492 (1991)
Reichmann, Reshaping human antibodies for therapy, Nature 332 (1998), 323-327.
Remington's Pharmaceutical Sciences 16th edition, Osol, A. Ed. (1980)
Ripka et al., Two Chinese hamster ovary glycosylation mutants affected in the conversion of GDP-mannose to GDP-fucose. Arch. Biochem. Biophys. 249:533-545 (1986).
Sambrook et al., Molecular Cloning: A Laboratory Manual; Cold Spring Harbor Laboratory Press, 2nd edition (1989) and 3rd edition (2001).
Scialó et al. TDP-43 real time quaking induced conversion reaction optimization and detection of seeding activity in CSF of amyotrophic lateral sclerosis and frontotemporal dementia patients. Brain Commun, fcaa142- (2020)
Shepherd and Dean (2000), Monoclonal Antibodies: A Practical Approach, Oxford University Press
Shields et al., High Resolution Mapping of the Binding Site on Human IgG1 for FcγRI, FcγRII, FcγRIII, and FcRn and Design of IgG1 Variants with Improved Binding to the FcTR, J. Biol. Chem. 9(2): 6591-6604 (2001).
Spiller K. J et al., Microglia-mediated recovery from ALS-relevant motor neuron degeneration in a mouse model of TDP-43 proteinopathy, Nature Neuroscience, 2018
Ticozzi et al., Protein Aggregation and Defective RNA Metabolism as Mechanisms for Motor Neuron Damage, 9(3): 285-296 CNS Neurol. Disord. Drug Targets. 2010, 9(3), 285-296.
Urlaub et al., Isolation of Chinese hamster cell mutants deficient in dihydrofolate reductase activity, Proc. Natl. Acad. cii. USA 77:4216 (1980)
Valente et al., Pharmacokinetics of novel Fc-engineered monoclonal and multispecific antibodies in cynomolgus monkeys and humanized FcRn transgenic mouse models, mAbs, 12:1, 1829337, DOI: 10.1080/19420862.2020.1829337
Verhoeyen et al., Reshaping human antibodies: Grafting an antilysozyme activity., Science 239 (1988), 1534-1536.
Walker et al., Neuropathologically mixed Alzheimer's and Lewy body disease: burden of pathological protein aggregates differs between clinical phenotypes, Acta Neuropathol (2015) 129:729-748
Wang A, Conicella A E, Schmidt H B, Martin E W, Rhoads S N, Reeb A N, et al. A single N-terminal phosphomimic disrupts TDP-43 polymerization, phase separation, and RNA splicing. EMBO J. 2018; 37 (5).
Wang et al., TDP-43: an emerging new player in neurodegenerative diseases Trends in Molecular Medicine Vol. 14 No. 11, 2008, 479-485.
Warraich et al., TDP-43: a DNA and RNA binding protein with roles in neurodegenerative diseases, The International Journal of Biochemistry & Cell Biology 42 (2010) 1606-1609.Wright et al., Effect of glycosylation on antibody function. TIBTECH 15:26-32 (1997).
Yamane-Ohnuki et al., Establishment of FUT8 knockout Chinese hamster ovary cells: An ideal host cell line for producing completely defucosylated antibodies with enhanced antibody-dependent cellular cytotoxicity. Biotech. Bioeng. 87: 614 (2004).
Yazaki and Wu, Methods in Molecular Biology, Val. 248 (B. K. C. Lo, ed., Humana Press, Totowa, NJ), pp. 255-268 (2003).
Zalevsky, J et al, Enhanced antibody half-life improves in vivo activity, Nat Biotechnol 28(2):157-9, 2010

1. A humanized TDP-43 binding molecule, in particular a humanized TDP-43 antibody or an antigen-binding fragment thereof, comprising:
a. a VH-CDR1 comprising the amino acid sequence of SEQ ID NO: 21;
b. a VH-CDR2 selected from the amino acid sequence of SEQ ID NO: 22, SEQ ID NO: 82, SEQ ID NO: 92; SEQ ID NO: 102, SEQ ID NO: 112 or SEQ ID NO: 122;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16;
f. a VL-CDR3 selected from the amino acid sequence of SEQ ID NO: 27 or SEQ ID NO:87; and:
g. an acceptor framework of a Heavy Chain Variable domain (VH) selected from the group consisting of IGHV1-69-2, IGHV5-51 or IGHV3-43, preferably IGHV1-69-2; and/or
h. an acceptor framework of a Light Chain Variable domain (VL) selected from the group consisting of IGKV2-28, IGKV2-24, IGKV2D-28, IGKV2-40, IGKV2D-40 or IGKV4-1, preferably IGKV2-28.

2. The humanized TDP-43 binding molecule of claim 1, wherein: the acceptor framework of the VH comprises one or more and preferably all of the following VH mutations:
a. V24T
b. Y27F
c. M48I
d. A71V
e. T73K; and
f. T94R;
and/or the acceptor framework of the VL comprises one or more of the following VL mutations:
g. I2V
h. Y36L
i. Q45K
j. L46R; and
k. G57R.

3. The humanized TDP-43 binding molecule of claim 2, wherein the acceptor framework of the VH comprises all of the following VH mutations:
a. V24T
b. Y27F
c. M48I
d. A71V
e. T73K; and
f. T94R;
and the acceptor framework of the VL comprises one or more, preferably all of the following VL mutations:
g. Y36L
h. L46R; and
i. G57R.

4. A humanized TDP-43 binding molecule, in particular a humanized TDP-43 antibody or an antigen-binding fragment thereof, optionally as defined in any one of claims 1 to 3, comprising:
a. a VH-CDR1 comprising the amino acid sequence of SEQ ID NO: 21;
b. a VH-CDR2 selected from the amino acid sequence of SEQ ID NO: 112, SEQ ID NO: 122 or SEQ ID NO: 102;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16; and
f. a VL-CDR3 selected from the amino acid sequence of SEQ ID NO: 27 or SEQ ID NO:87.

5. The humanized TDP-43 binding molecule of any one of the preceding claims, in particular a humanized TDP-43 antibody or an antigen-binding fragment thereof, comprising:
a. a VH-CDR1 comprising the amino acid sequence of SEQ ID NO: 21;
b. a VH-CDR2 selected from the amino acid sequence of SEQ ID NO: 112 or SEQ ID NO: 122;
c. a VH-CDR3 comprising the amino acid sequence ES (Glu-Ser);
d. a VL-CDR1 selected from the amino acid sequence of SEQ ID NO: 25 or SEQ ID NO: 85;
e. a VL-CDR2 selected from the amino acid sequence of SEQ ID NO: 16; and
f. a VL-CDR3 selected from the amino acid sequence of SEQ ID NO: 27 or SEQ ID NO:87.

6. The humanized TDP-43 binding molecule of any one of the preceding claims, which comprises:
a. a Heavy Chain Variable domain (VH) which comprises a VH-CDR1 comprising the amino acid sequence of SEQ ID NO: 21; a VH-CDR2 comprising the amino acid sequence of SEQ ID NO: 22; a VH-CDR3 comprising the amino acid sequence ES (Glu-Ser); and a Light Chain Variable domain (VL) which comprises a VL-CDR1 comprising the amino acid sequence of SEQ ID NO: 25; a VL-CDR2 comprising the amino acid sequence of SEQ ID NO: 16; and a VL-CDR3 comprising the amino acid sequence of SEQ ID NO: 27; or
b. a Heavy Chain Variable domain (VH) which comprises a VH-CDR1 comprising the amino acid sequence of SEQ ID NO: 21; a VH-CDR2 comprising the amino acid sequence of SEQ ID NO: 112; a VH-CDR3 comprising the amino acid sequence ES (Glu-Ser); and a Light Chain Variable domain (VL) which comprises a VL-CDR1 comprising the amino acid sequence of SEQ ID NO: 25; a VL-CDR2 comprising the amino acid sequence of SEQ ID NO: 16; and a VL-CDR3 comprising the amino acid sequence of SEQ ID NO: 27; or
c. a Heavy Chain Variable domain (VH) which comprises a VH-CDR1 comprising the amino acid sequence of SEQ ID NO: 21; a VH-CDR2 comprising the amino acid sequence of SEQ ID NO: 112; a VH-CDR3 comprising the amino acid sequence ES (Glu-Ser); and a Light Chain Variable domain (VL) which comprises a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
d. a Heavy Chain Variable domain (VH) which comprises a VH-CDR1 comprising the amino acid sequence of SEQ ID NO: 21; a VH-CDR2 comprising the amino acid sequence of SEQ ID NO: 122; a VH-CDR3 comprising the amino acid sequence ES (Glu-Ser); and a Light Chain Variable domain (VL) which comprises a VL-CDR1 comprising the amino acid sequence of SEQ ID NO: 25; a VL-CDR2 comprising the amino acid sequence of SEQ ID NO: 16; and a VL-CDR3 comprising the amino acid sequence of SEQ ID NO: 27; or
e. a Heavy Chain Variable domain (VH) which comprises a VH-CDR1 comprising the amino acid sequence of SEQ ID NO: 21; a VH-CDR2 comprising the amino acid sequence of SEQ ID NO: 122; a VH-CDR3 comprising the amino acid sequence ES (Glu-Ser); and a Light Chain Variable domain (VL) which comprises a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
f. a Heavy Chain Variable domain (VH) which comprises a VH-CDR1 comprising the amino acid sequence of SEQ ID NO: 21; a VH-CDR2 comprising the amino acid sequence of SEQ ID NO: 112; a VH-CDR3 comprising the amino acid sequence ES (Glu-Ser); and a Light Chain Variable domain (VL) which comprises a VL-CDR1 comprising the amino acid sequence of SEQ ID NO: 25; a VL-CDR2 comprising the amino acid sequence of SEQ ID NO: 16; and a VL-CDR3 comprising the amino acid sequence of SEQ ID NO: 87; or
g. a Heavy Chain Variable domain (VH) which comprises a VH-CDR1 comprising the amino acid sequence of SEQ ID NO: 21; a VH-CDR2 comprising the amino acid sequence of SEQ ID NO: 122; a VH-CDR3 comprising the amino acid sequence ES (Glu-Ser); and a Light Chain Variable domain (VL) which comprises a VL-CDR1 comprising the amino acid sequence of SEQ ID NO: 25; a VL-CDR2 comprising the amino acid sequence of SEQ ID NO: 16; and a VL-CDR3 comprising the amino acid sequence of SEQ ID NO: 87.

7. The humanized TDP-43 binding molecule of any one of the preceding claims:
a. wherein the acceptor framework of the Heavy Chain Variable domain (VH) is selected from the group consisting of IGHV1-69-2, IGHV5-51 or IGHV3-43, preferably IGHV1-69-2; and
b. wherein the acceptor framework of the Light Chain Variable domain (VL) is selected from the group consisting of IGKV2-28, IGKV2-24, IGKV2D-28, IGKV2-40, IGKV2D-40 or IGKV4-1, preferably IGKV2-28.

8. The humanized TDP-43 binding molecule of claim 7:
a. wherein the acceptor framework of the Heavy Chain Variable domain (VH) is IGHV1-69-2; and
b. wherein the acceptor framework of the Light Chain Variable domain (VL) is IGKV2-28.

9. The humanized TDP-43 binding molecule of any one of the preceding claims, which comprises:
a. a Heavy Chain Variable Region (VH) selected from:
i. a Heavy Chain Variable Region (VH) comprising the sequence of SEQ ID NO: 30 or a Heavy Chain Variable Region (VH) having at least 91%, 92%, 93%, 94%, 95%, 96%, 97%, 98% or 99% sequence identity to the amino acid sequence of SEQ ID NO: 30; or
ii. a Heavy Chain Variable Region (VH) comprising the sequence of SEQ ID NO: 40 or a Heavy Chain Variable Region (VH) having at least 91%, 92%, 93%, 94%, 95%, 96%, 97%, 98% or 99% sequence identity to the amino acid sequence of SEQ ID NO: 40; or
iii. a Heavy Chain Variable Region (VH) comprising the sequence of SEQ ID NO: 50 or a Heavy Chain Variable Region (VH) having at least 91%, 92%, 93%, 94%, 95%, 96%, 97%, 98%, or 99% sequence identity to the amino acid sequence of SEQ ID NO: 50; or
iv. a Heavy Chain Variable Region (VH) comprising the sequence of SEQ ID NO: 60 or a Heavy Chain Variable Region (VH) having at least 91%, 92%, 93%, 94%, 95%, 96%, 97%, 98% or 99% sequence identity to the amino acid sequence of SEQ ID NO: 60; or
v. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; or
vi. a Heavy Chain Variable Region (VH) comprising the sequence of SEQ ID NO: 80 or a Heavy Chain Variable Region (VH) having at least 91%, 92%, 93%, 94%, 95%, 96%, 97%, 98% or 99% sequence identity to the amino acid sequence of SEQ ID NO: 80; or
vii. a Heavy Chain Variable Region (VH) comprising the sequence of SEQ ID NO: 90 or a Heavy Chain Variable Region (VH) having at least 91%, 92%, 93%, 94%, 95%, 96%, 97%, 98% or 99% sequence identity to the amino acid sequence of SEQ ID NO: 90; or
viii. a Heavy Chain Variable Region (VH) comprising the sequence of SEQ ID NO: 100 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 100; or
ix. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; or
x. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and
b. a Light Chain Variable Region (VL) selected from:
i. a Light Chain Variable Region (VL) comprising the sequence of SEQ ID NO: 34 or a Light Chain Variable Region (VL) having at least 97%, 98% or 99% sequence identity to the amino acid sequence of SEQ ID NO: 34; or
ii. a Light Chain Variable Region (VL) comprising the sequence of SEQ ID NO: 44 or a Light Chain Variable Region (VL) having at least 96%, 97%, 98% or 99% sequence identity to the amino acid sequence of SEQ ID NO: 44; or
iii. a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
iv. a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
v. a Light Chain Variable Region (VL) comprising the sequence of SEQ ID NO: 74 or a Light Chain Variable Region (VL) having at least 96%, 97%, 98% or 99% sequence identity to the amino acid sequence of SEQ ID NO: 74; or
vi. a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
vii. a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94.

10. The humanized TDP-43 binding molecule of any one of the preceding claims, which comprises:
a. a Heavy Chain Variable Region (VH) comprising the sequence of SEQ ID NO: 60 or a Heavy Chain Variable Region (VH) having at least 91%, 92%, 93%, 94%, 95%, 96%, 97%, 98% or 99% sequence identity to the amino acid sequence of SEQ ID NO: 60; and a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
b. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the sequence of SEQ ID NO: 44 or a Light Chain Variable Region (VL) having at least 96%, 97%, 98% or 99% sequence identity to the amino acid sequence of SEQ ID NO: 44; or
c. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the sequence of SEQ ID NO: 54 or a Light Chain Variable Region (VL) having at least 98% or 99% sequence identity to the amino acid sequence of SEQ ID NO: 54; or
d. a Heavy Chain Variable Region (VH) comprising the sequence of SEQ ID NO: 70 or a Heavy Chain Variable Region (VH) having at least 90%, 91%, 92%, 93%, 94%, 95%, 96%, 97%, 98% or 99% sequence identity to the amino acid sequence of SEQ ID NO: 70; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
e. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
f. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
g. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and a Light Chain Variable Region (VL) comprising the sequence of SEQ ID NO: 64 or a Light Chain Variable Region (VL) having at least 97%, 98% or 99% sequence identity to the amino acid sequence of SEQ ID NO: 64; or
h. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and a Light Chain Variable Region (VL) comprising the sequence of SEQ ID NO: 84 or a Light Chain Variable Region (VL) having at least 97%, 98% or 99% sequence identity to the amino acid sequence of SEQ ID NO: 84; or
i. a Heavy Chain Variable Region (VH) comprising the sequence of SEQ ID NO: 110 or a Heavy Chain Variable Region (VH) having at least 89%, 90%, 91%, 92%, 93%, 94%, 95%, 96%, 97%, 98% or 99% sequence identity to the amino acid sequence of SEQ ID NO: 110; and a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94; or
j. a Heavy Chain Variable Region (VH) comprising the sequence of SEQ ID NO: 120 or a Heavy Chain Variable Region (VH) having at least 91%, 92%, 93%, 94%, 95%, 96%, 97%, 98% or 99% sequence identity to the amino acid sequence of SEQ ID NO: 120; and a Light Chain Variable Region (VL) comprising the sequence of SEQ ID NO: 94 or a Light Chain Variable Region (VL) having at least 96%, 97%, 98% or 99% sequence identity to the amino acid sequence of SEQ ID NO: 94.

11. The humanized TDP-43 binding molecule of any one of the preceding claims, which comprises:
a. a Heavy Chain Variable Region (VH) comprising the sequence of SEQ ID NO: 60 and a Light Chain Variable Region (VL) comprising the sequence of SEQ ID NO: 54; or
b. a Heavy Chain Variable Region (VH) comprising the sequence of SEQ ID NO: 70 and a Light Chain Variable Region (VL) comprising the sequence of SEQ ID NO: 44; or
c. a Heavy Chain Variable Region (VH) comprising the sequence of SEQ ID NO: 70 and a Light Chain Variable Region (VL) comprising the sequence of SEQ ID NO: 54; or
d. a Heavy Chain Variable Region (VH) comprising the sequence of SEQ ID NO: 70 and a Light Chain Variable Region (VL) comprising the sequence of SEQ ID NO: 64; or
e. a Heavy Chain Variable Region (VH) comprising the sequence of SEQ ID NO: 110 and a Light Chain Variable Region (VL) comprising the sequence of SEQ ID NO: 64; or
f. a Heavy Chain Variable Region (VH) comprising the sequence of SEQ ID NO: 110 and a Light Chain Variable Region (VL) comprising the sequence of SEQ ID NO: 84; or
g. a Heavy Chain Variable Region (VH) comprising the sequence of SEQ ID NO: 120 and a Light Chain Variable Region (VL) comprising the sequence of SEQ ID NO: 64; or
h. a Heavy Chain Variable Region (VH) comprising the sequence of SEQ ID NO: 120 and a Light Chain Variable Region (VL) comprising the sequence of SEQ ID NO: 84; or
i. a Heavy Chain Variable Region (VH) comprising the sequence of SEQ ID NO: 110 and a Light Chain Variable Region (VL) comprising the sequence of SEQ ID NO: 94; or
j. a Heavy Chain Variable Region (VH) comprising the sequence of SEQ ID NO: 120 and a Light Chain Variable Region (VL) comprising the sequence of SEQ ID NO: 94.

12. The humanized TDP-43 binding molecule of any one of the preceding claims, which comprises:
a. a Heavy Chain Variable Region (VH) comprising the sequence of SEQ ID NO: 120 and a Light Chain Variable Region (VL) comprising the sequence of SEQ ID NO: 84; or
b. a Heavy Chain Variable Region (VH) comprising the sequence of SEQ ID NO: 110 and a Light Chain Variable Region (VL) comprising the sequence of SEQ ID NO: 94; or
c. a Heavy Chain Variable Region (VH) comprising the sequence of SEQ ID NO: 120 and a Light Chain Variable Region (VL) comprising the sequence of SEQ ID NO: 94.

13. The humanized TDP-43 binding molecule of any one of the preceding claims, which binds misfolded aggregated TDP-43 and non-aggregated physiological TDP-43.

14. The humanized TDP-43 binding molecule of any one of the preceding claims, which binds to monomeric and/or oligomeric and/or aggregated and/or post-translationally modified and/or truncated TDP-43, preferably human TDP-43.

15. The humanized TDP-43 binding molecule of any one of the preceding claims, which binds to misfolded aggregated human TDP-43 and non-aggregated physiological human TDP-43.

16. The humanized TDP-43 binding molecule of any one of the preceding claims, which exhibits one or more, up to all of the following characteristics:
a. inhibits the aggregation of TDP-43 protein or fragments thereof,
b. blocks TDP-43 cell-to-cell propagation;
c. disaggregates TDP-43 aggregates;
d. blocks TDP-43 seeding;
e. neutralizes seeding-competent TDP-43;
f. blocks TDP-43 spreading; and
g. potentiates TDP-43 clearance.

17. The humanized TDP-43 binding molecule of any one of the preceding claims, which neutralizes seeding-competent TDP-43.

18. The humanized TDP-43 binding molecule of any one of the preceding claims, which potentiates TDP-43 clearance.

19. The humanized TDP-43 binding molecule of any one of the preceding claims, which reduces TDP-43 pathology in vivo.

20. The humanized TDP-43 binding molecule of any one of the preceding claims, which reduces levels of misfolded aggregated TDP-43 and/or phosphorylated TDP-43 in vivo.

21. The humanized TDP-43 binding molecule of any one of the preceding claims, which binds to an epitope within amino acids residues 397-411 of human TDP-43 (SEQ ID NO: 1).

22. The humanized TDP-43 binding molecule of any one of the preceding claims, which is an antibody or an antigen-binding fragment thereof.

23. The humanized TDP-43 binding molecule of any one of the preceding claims, which has a dissociation constant (KD) of less than 1 nM for soluble TDP-43 (SEQ ID NO: 1) binding, preferably less than 250 pM.

24. The humanized TDP-43 binding molecule of any one of the preceding claims, which has a dissociation constant (KD) of less than 4 nM for soluble TP-51 (amino acids 352-414 of SEQ ID NO: 1) peptide binding, preferably less than 2 nM.

25. The humanized TDP-43 binding molecule of any one of the preceding claims, which is an IgA, IgD, IgE, IgM, IgG1, IgG2, IgG3 or IgG4 antibody or antigen-binding fragment thereof.

26. The humanized TDP-43 binding molecule of any one of the preceding claims, which is an IgG1 or an IgG4 antibody or antigen-binding fragment thereof.

27. The humanized TDP-43 binding molecule of any one of the preceding claims which comprises an Fc mutation, preferably the S228P mutation.

28. The humanized TDP-43 binding molecule of any one of the preceding claims which comprises a Fv having a pI below 7.5, preferably below 7.0.

29. The humanized TDP-43 binding molecule of any one of the preceding claims which comprises a Fv having a net charge, at pH 7.4, below 0.2, preferably below −0.8, more preferably below −1.8.

30. An immunoconjugate comprising the humanized TDP-43 binding molecule according to any one of the preceding claims.

31. An immunoconjugate comprising the humanized TDP-43 binding molecule according to any one of claims 1 to 29, wherein the immunoconjugate crosses the blood brain barrier using a delivery vehicle or a blood brain barrier moiety.

32. The immunoconjugate of claim 30 or 31, wherein the delivery vehicle comprises a liposome or extracellular vesicle.

33. The immunoconjugate of claim 30 or 31, wherein the humanized TDP-43 binding molecule is linked to a blood brain barrier moiety.

34. The immunoconjugate of claim 33, wherein the blood brain barrier moiety is a polypeptide or a small molecule, preferably, a peptide, a receptor ligand, a single domain antibody (VHH), a scFv or a Fab fragment.

35. The immunoconjugate of claim 31, 33 or 34, wherein the blood brain barrier moiety binds a blood brain barrier receptor.

36. The immunoconjugate of claim 35, wherein blood brain barrier receptor includes, but is not limited to transferrin receptor, insulin receptor or low-density lipoprotein receptor.

37. A labeled binding molecule, in particular a labelled antibody, comprising the humanized TDP-43 binding molecule according to any one of claims 1 to 29.

38. A pharmaceutical composition comprising the humanized TDP-43 binding molecule of any one of claims 1 to 29, or an immunoconjugate as claimed in any one of claims 30 to 36 and a pharmaceutically acceptable carrier and/or excipient and/or diluent.

39. The humanized TDP-43 binding molecule of any one of claims 1 to 29, or an immunoconjugate as claimed in any one of claims 30 to 36 or pharmaceutical composition of claim 38 for human or veterinary medicine use.

40. The humanized TDP-43 binding molecule of any one of claims 1 to 29, or an immunoconjugate as claimed in any one of claims 30 to 36 or pharmaceutical composition of claim 38 for use in the prevention, alleviation, treatment of diseases, disorders and/or abnormalities associated with TDP-43 or TDP-43 proteinopathy.

41. The humanized TDP-43 binding molecule of any one of claims 1 to 29 or an immunoconjugate as claimed in any one of claims 30 to 36, labeled binding molecule of claim 37 or pharmaceutical composition of claim 38 for diagnostic use.

42. The humanized TDP-43 binding molecule or immunoconjugate, labeled binding molecule or pharmaceutical composition for use as claimed in claim 41 for diagnosis of diseases, disorders and/or abnormalities associated with TDP-43 or TDP-43 proteinopathy.

43. The humanized TDP-43 binding molecule of any one of claims 1 to 29 or an immunoconjugate as claimed in any one of claims 30 to 36, labeled binding molecule of claim 37 or pharmaceutical composition of claim 38 for research use, in particular as an analytical tool or reference molecule.

44. The humanized TDP-43 binding molecule of any one of claims 1 to 29 or an immunoconjugate as claimed in any one of claims 30 to 36, labeled binding molecule of claim 37 or pharmaceutical composition of claim 38 for use as a diagnostic tool to monitor diseases, disorders and/or abnormalities associated with TDP-43 or TDP-43 proteinopathy.

45. The humanized TDP-43 binding molecule or immunoconjugate or labeled binding molecule or pharmaceutical composition for use according to any one of claims 40, 42 or 44, wherein the disease, disorder and/or abnormality associated with TDP-43, or TDP-43 proteinopathy, is Frontotemporal dementia (FTD, such as sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) with ubiquitin-positive TDP-43 inclusions (FTLD-TDP), Argyrophilic grain disease, Pick's disease, semantic variant Primary Progressive Aphasia (svPPA), behavioural variant FTD (bvFTD), nonfluent variant Primary Progressive Aphasia (nfvPPA) and the like), Amyotrophic lateral sclerosis (ALS, such as sporadic ALS, with TARDBP mutation, with angiogenin (ANG) mutation), Alexander disease (AxD), limbic-predominant age-related TDP-43 encephalopathy (LATE), Chronic Traumatic Encephalopathy (CTE), Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (sporadic inclusion body myositis, Inclusion body myopathy with a mutation in the valosin-containing protein ((VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES)), Traumatic Brain Injury (TBI), Dementia with Lewy Bodies (DLB) or Parkinson's disease (PD).

46. The humanized TDP-43 binding molecule or immunoconjugate or labeled binding molecule or pharmaceutical composition for use according to claim 45, wherein the disease, disorder and/or abnormality associated with TDP-43, or TDP-43 proteinopathy, is Frontotemporal dementia (FTD), amyotrophic lateral sclerosis (ALS), Alzheimer's disease (AD), Parkinson's disease (PD), Chronic Traumatic Encephalopathy (CTE), or limbic-predominant age-related TDP-43 encephalopathy (LATE).

47. The humanized TDP-43 binding molecule or immunoconjugate or labeled binding molecule or pharmaceutical composition for use according to claim 46, wherein the disease, disorder and/or abnormality associated with TDP-43, or TDP-43 proteinopathy, is amyotrophic lateral sclerosis (ALS).

48. The humanized TDP-43 binding molecule or immunoconjugate or labeled binding molecule or pharmaceutical composition for use according to claim 46, wherein the disease, disorder and/or abnormality associated with TDP-43, or TDP-43 proteinopathy, is Alzheimer's disease (AD).

49. The humanized TDP-43 binding molecule or immunoconjugate or labeled binding molecule or pharmaceutical composition for use according to claim 46, wherein the disease, disorder, and/or abnormality associated with TDP-43, or TDP-43 proteinopathy, is Frontotemporal dementia (FTD).

50. A method of retaining or increasing cognitive memory capacity or slowing memory loss in an individual with a disease, disorder and/or abnormality associated with TDP-43 or a TDP-43 proteinopathy, comprising administering the humanized TDP-43 binding molecule of any one of claims 1 to 29 or an immunoconjugate as claimed in any one of claims 30 to 36 or pharmaceutical composition of claim 38 to the individual.

51. A method of reducing the level of aggregated TDP-43 and/or phosphorylated TDP-43 in an individual, comprising administering the humanized TDP-43 binding molecule of claims 1 to 29 or an immunoconjugate as claimed in any one of claims 30 to 36 or pharmaceutical composition of claim 38 to the individual.

52. The method of claim 50 or 51, wherein the method comprises administering at least one additional therapeutic agent.

53. The method of claim 52, wherein the additional therapeutic agent targets alpha-synuclein, BACE1, Tau, beta-amyloid, TDP-43 or a neuroinflammation protein.

54. A nucleic acid molecule encoding the humanized TDP-43 binding molecule of any one of claims 1 to 29.

55. A nucleic acid molecule encoding a humanized TDP-43 binding molecule comprising a nucleotide sequence as provided in SEQ ID NO: 38, SEQ ID NO: 48, SEQ ID NO: 58, SEQ ID NO: 68, SEQ ID NO: 78, SEQ ID NO: 88, SEQ ID NO: 98, SEQ ID NO: 108, SEQ ID NO: 118, SEQ ID NO: 128, SEQ ID NO: 39, SEQ ID NO: 49, SEQ ID NO: 59, SEQ ID NO: 69, SEQ ID NO: 79, SEQ ID NO: 89, or SEQ ID NO: 99.

56. A nucleic acid molecule encoding a humanized TDP-43 binding molecule comprising a nucleotide sequence set forth as:
a. a Heavy Chain Variable Region (VH) encoded by SEQ ID NO: 68 and a Light Chain Variable Region (VL) encoded by SEQ ID NO: 59; or
b. a Heavy Chain Variable Region (VH) encoded by SEQ ID NO: 78 and a Light Chain Variable Region (VL) encoded by SEQ ID NO: 49; or
c. a Heavy Chain Variable Region (VH) encoded by SEQ ID NO: 78 and a Light Chain Variable Region (VL) encoded by SEQ ID NO: 59; or
d. a Heavy Chain Variable Region (VH) encoded by SEQ ID NO: 78 and a Light Chain Variable Region (VL) encoded by SEQ ID NO: 69; or
e. a Heavy Chain Variable Region (VH) encoded by SEQ ID NO: 118 and a Light Chain Variable Region (VL) encoded by SEQ ID NO: 69; or
f. a Heavy Chain Variable Region (VH) encoded by SEQ ID NO: 118 and a Light Chain Variable Region (VL) encoded by SEQ ID NO: 89; or
g. a Heavy Chain Variable Region (VH) encoded by SEQ ID NO: 128 and a Light Chain Variable Region (VL) encoded by SEQ ID NO: 69; or
h. a Heavy Chain Variable Region (VH) encoded by SEQ ID NO: 128 and a Light Chain Variable Region (VL) encoded by SEQ ID NO: 89; or
i. a Heavy Chain Variable Region (VH) encoded by SEQ ID NO: 118 and a Light Chain Variable Region (VL) encoded by SEQ ID NO: 99; or
j. a Heavy Chain Variable Region (VH) encoded by SEQ ID NO: 128 and a Light Chain Variable Region (VL) encoded by SEQ ID NO: 99.

57. A nucleic acid according to any one of claims 54 to 56, wherein the nucleic acid is a part of a viral vector for targeted delivery to the blood brain barrier or any other cell type in the CNS.

58. The nucleic acid according to claim 57, wherein the viral vector is a recombinant adeno-associated viral vector (rAAV), preferably a recombinant adeno-associated viral vector selected from AAV1 to AAV12.

59. A recombinant expression vector comprising the nucleic acid of any one of claims 54 to 58.

60. A host cell comprising the nucleic acid of any one of claims 54 to 58 and/or the vector of claim 59.

61. A host cell that expresses a humanized TDP-43 binding molecule according to any one of claims 1 to 29.

62. A cell-free expression system containing the recombinant expression vector of claim 59.

63. A method for producing a humanized TDP-43 binding molecule, in particular a humanized antibody or antigen-binding fragment thereof, comprising the steps of:
a. culturing the host cell of claim 60 or 61 or cell-free expression system of claim 62 under conditions suitable for producing the humanized TDP-43 binding molecule, in particular the humanized antibody or antigen-binding fragment thereof; and
b. isolating the humanized TDP-43 binding molecule, in particular the humanized antibody or antigen-binding fragment thereof.

64. A method of detecting and/or quantifying TDP-43 in a sample obtained from a subject, the method comprising contacting the sample with a humanized TDP-43 binding molecule according to any one of claims 1 to 29 and comparing the TDP-43 levels in the sample to those in a control sample or samples.

65. The method of claim 64, wherein the sample is human blood, cerebrospinal fluid (CSF), interstitial fluid (ISF) and/or urine, preferably CSF.

66. A kit for diagnosis of a disease, disorder and/or abnormality associated with TDP-43, or a TDP-43 proteinopathy, or for use according to any one of claims 39 to 49, or for use in a method of any one of claims 50 to 53 comprising a humanized TDP-43 binding molecule according to any one of claims 1 to 29.
